SEQUENCE LISTING (203 sequences)

SEQ ID NO: 1 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 169 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 4 - protein, 169 aa, synthetic construct
KSKAVEQVKNAFNKLQEKGIYKAMSEFDIFINYIEAYMTMKIRNTGSGGGGSGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 5 - protein, 169 aa, synthetic construct
KSKAVEQVKNAFNKLQEKGIYKAMSEFDIFINYIEAYMTMKIRNGGSGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 6 - protein, 168 aa, synthetic construct
KSKAVEQVKNAFNKLQEKGIYKAMSEFDIFINYIEAYMTMKIRNGGSGGGSGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 7 - protein, 175 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENHHHHHH

SEQ ID NO: 8 - protein, 166 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 9 - protein, 172 aa, synthetic construct
HHHHHHSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 10 - protein, 129 aa, Homo sapiens [sequence omitted]

SEQ ID NO: 11 - protein, 113 aa, Homo sapiens
HKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 12 - protein, 129 aa, Homo sapiens
HKRDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 13 - protein, 113 aa, Homo sapiens
HKRDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 14 - protein, 114 aa, Homo sapiens
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 15 - protein, 113 aa, Homo sapiens
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 16 - protein, 112 aa, Homo sapiens
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 17 - protein, 114 aa, Homo sapiens
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 18 - protein, 122 aa, Homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 19 - protein, 113 aa, Homo sapiens
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 20 - protein, 112 aa, Homo sapiens
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 21 - protein, 122 aa, Homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 22 - protein, 9 aa, synthetic construct
GSGGGGSGT

SEQ ID NO: 23 - protein, 9 aa, synthetic construct
TGSGGGGSG

SEQ ID NO: 24 - protein, 9 aa, synthetic construct
GGSGGGSGT

SEQ ID NO: 25 - protein, 8 aa, synthetic construct
GSGGGGSG

SEQ ID NO: 26 - protein, 8 aa, synthetic construct
GGSGGGSG

SEQ ID NO: 27 - skipped

SEQ ID NO: 28 - protein, 4 aa, synthetic construct
GGGS

SEQ ID NO: 29 - protein, 5 aa, synthetic construct
GGGGS

SEQ ID NO: 30 - protein, 15 aa, synthetic construct
GGGGSGGGGSGGGGS

SEQ ID NO: 31 - protein, 18 aa, synthetic construct
KESGSVSSEQLAQFRSLD

SEQ ID NO: 32 - protein, 14 aa, synthetic construct
EGKSSGSGSESKST

SEQ ID NO: 33 - protein, 12 aa, synthetic construct
GSAGSAAGSGEF

SEQ ID NO: 34 - protein, 7 aa, synthetic construct
GGGGSAS

SEQ ID NO: 35 - protein, 12 aa, synthetic construct
GGGGSGGGGSAS

SEQ ID NO: 36 - protein, 17 aa, synthetic construct
GGGGSGGGGSGGGGSAS

SEQ ID NO: 37 - protein, 27 aa, synthetic construct
GGGGSGGGGSGGGGSGGGGSGGGGSAS

SEQ ID NO: 38 - protein, 32 aa, synthetic construct
GGGGSGGGGSGGGGSGGGGSGGGGSGGGGSAS

SEQ ID NO: 39 - protein, 37 aa, synthetic construct
AGGGSGGGGSGGGGSGGGGSGGGGSGGGGSGGGGSAS

SEQ ID NO: 40 - protein, 42 aa, synthetic construct
AGGGSGGGGSGGGGSGGGGSGGGGSGGGGSGGGGSGGGGSAS

SEQ ID NO: 41 - protein, 47 aa, synthetic construct
AGGGSGGGGSGGGGSGGGGSGGGGSGGGGSGGGGSGGGGSGGGGSAS

SEQ ID NO: 42 - protein, 5 aa, synthetic construct
EAAAK

SEQ ID NO: 43 - protein, 5 aa, synthetic construct
EAAAR

SEQ ID NO: 44 - protein, 5 aa, synthetic construct
PAPAP

SEQ ID NO: 45 - protein, 12 aa, synthetic construct
AEAAAKEAAAKA

SEQ ID NO: 46 - protein, 27 aa, synthetic construct
SGGGSDPAEPKSPDKTHTCPPCPKDPK

SEQ ID NO: 47 - protein, 20 aa, synthetic construct
AEPKSPDKTHTCPPCPKDPK

SEQ ID NO: 48 - protein, 45 aa, synthetic construct
TTTPAPRPPTPAPTIASQPLSLRPEACRPAAGGAVHTRGLDFACD

SEQ ID NO: 49 - protein, 74 aa, synthetic construct
AGSGGSGGSGGSPVPSTPPTNSSSTPPTPSPSPVPSTPPTNSSSTPPTPSPSPVPSTPPTNSSSTPPTPSPSAS

SEQ ID NO: 50 - protein, 74 aa, synthetic construct
AGSGGSGGSGGSPVPSTPPTPSPSTPPTPSPSPVPSTPPTNSSSTPPTPSPSPVPSTPPTPSPSTPPTPSPSAS

SEQ ID NO: 51 - protein, 66 aa, synthetic construct
AGSGGSGGSGGSPVPSTPPTPSPSTPPTPSPSGGSGNSSGSGGSPVPSTPPTPSPSTPPTPSPSAS

SEQ ID NO: 52 - protein, 74 aa, synthetic construct
AGSGGSGGSGGSPVPSTPPTPSPSTPPTPSPSPVPSTPPTPSPSTPPTPSPSPVPSTPPTPSPSTPPTPSPSAS

SEQ ID NO: 53 - protein, 152 aa, synthetic construct
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPSAS

SEQ ID NO: 54 - protein, 144 aa, synthetic construct
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSAS

SEQ ID NO: 55 - protein, 242 aa, synthetic construct
AGPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGAS

SEQ ID NO: 56 - protein, 262 aa, synthetic construct
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPSAS

SEQ ID NO: 57 - protein, 121 aa, synthetic construct
AGSGGSGGSGGSPVPSTPPTPSPSTPPTPSPSQIFVKTLTGKTITLEVEPSDTIENVKAKIQDKEGIPPDQQRLIFAGKQLEDGRTLSDYNIQKESTLHLVLRLRGGGGSGGSGGSGGSAS

SEQ ID NO: 58 - protein, 326 aa, synthetic construct
AGSGGSGGSGGSPVPSTPPTPSPSTPPTPSPSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPSAS

SEQ ID NO: 59 - protein, 606 aa, synthetic construct
AGSGGSGGSGGSGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPSPSAS

SEQ ID NO: 60 - protein, 46 aa, synthetic construct
AGSGNSSGSGGSGGSGNSSGSGGSPVPSTPPTPSPSTPPTPSPSAS

SEQ ID NO: 61 - protein, 141 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 62 - protein, 245 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGAS

SEQ ID NO: 63 - protein, 349 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 64 - protein, 451 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAGGGGSGGGGSGGGGAS

SEQ ID NO: 65 - protein, 307 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGSGGGGSGTHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 66 - protein, 313 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGSGGGGSGTHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSHHHHHH

SEQ ID NO: 67 - protein, 304 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 68 - protein, 310 aa, synthetic construct
HHHHHHHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 69 - protein, 307 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTKSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 70 - protein, 304 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNGSGGGGSGTHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 71 - protein, 292 aa, synthetic construct
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGSGGGGSGTKSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 72 - protein, 292 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGSGGGGSGTSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 73 - protein, 289 aa, synthetic construct
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 74 - protein, 289 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNGSGGGGSGTSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 75 - protein, 330 aa, Homo sapiens [sequence omitted]

SEQ ID NO: 76 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 77 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 78 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 428 aa, Homo sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 80 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 81 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 82 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 83 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 84 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 85 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 86 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 12 aa, Homo sapiens
EPKSCDKTHTCP

SEQ ID NO: 88 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 90 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 91 - protein, 107 aa, Homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 92 - protein, 12 aa, Homo sapiens
ERKCCVECPPCP

SEQ ID NO: 93 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 94 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 95 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 96 - protein, 106 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 97 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 98 - protein, 216 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 99 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 100 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 101 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 12 aa, Homo sapiens
ESKYGPPCPSCP

SEQ ID NO: 103 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 106 aa, unidentified
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 106 aa, unidentified
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 106 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 360 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 109 - protein, 346 aa, synthetic construct
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNTGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 400 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 397 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 360 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGSGGGGSGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 113 - protein, 360 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 346 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKTGSGGGGSGSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 115 - protein, 346 aa, synthetic construct
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNTGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 116 - protein, 400 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 117 - protein, 400 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGKSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEN

SEQ ID NO: 118 - protein, 397 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNGSGGGGSGTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 119 - protein, 298 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 120 - protein, 258 aa, synthetic construct
HHHHHHHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 121 - protein, 19 aa, synthetic construct
MGWTLVFLFLLSVTAGVHS

SEQ ID NO: 122 - protein, 20 aa, synthetic construct
MVSSAQFLGLLLLCFQGTRC

SEQ ID NO: 123 - protein, 6 aa, synthetic construct
HHHHHH

SEQ ID NO: 124 - protein, 6 aa, synthetic construct
GGGSGG

SEQ ID NO: 125 - protein, 215 aa, unidentified
FPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 126 - protein, 215 aa, synthetic construct
FPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNCPEEEEEEEEEEEEERKGLLPGALGSALQGPAQVSWPQLLSTYRLLHSLELVLSRAVRELLLLSKAGHSVWPLGFPTLSPQP

SEQ ID NO: 127 - protein, 209 aa, unidentified
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 128 - protein, 270 aa, unidentified
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 129 - protein, 176 aa, unidentified
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 130 - protein, 172 aa, unidentified
SGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 131 - protein, 162 aa, unidentified
HDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 132 - protein, 228 aa, unidentified
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 133 - protein, 228 aa, unidentified
MKPKMKYSTNKISTAKWKNTASKALCFKLGKSQQKAKEVCPMYFMKLRSGLMIKKEACYFRRETTKRPSLKTGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 134 - protein, 144 aa, unidentified
MKPKMKYSTNKISTAKWKNTASKALCFKLGNKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 135 - protein, 390 aa, unidentified
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 136 - protein, 112 aa, unidentified
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 137 - protein, 442 aa, unidentified
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 138 - protein, 414 aa, unidentified
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 139 - protein, 112 aa, unidentified
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 140 - protein, 24 aa, Homo sapiens
NLPNMLRDLRDAFSRVKTFFQMKD

SEQ ID NO: 141 - protein, 10 aa, Homo sapiens
KESLLEDFKG

SEQ ID NO: 142 - protein, 23 aa, Homo sapiens
LGCQALSEMIQFYLEEVMPQAEN

SEQ ID NO: 143 - protein, 22 aa, Homo sapiens
IKAHVNSLGENLKTLRLRLRRC

SEQ ID NO: 144 - protein, 14 aa, Homo sapiens
SKAVEQVKNAFNKL

SEQ ID NO: 145 - protein, 27 aa, Homo sapiens
EKGIYKAMSEFDIFINYIEAYMTMKIR

SEQ ID NO: 146 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 102 aa, Homo sapiens
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 148 - protein, 23 aa, Homo sapiens
EPKSCDKTHTCPPCPAPELLGGP

SEQ ID NO: 149 - protein, 10 aa, Homo sapiens
EPKSCDKTHT

SEQ ID NO: 150 - protein, 5 aa, Homo sapiens
CPPCP

SEQ ID NO: 151 - protein, 8 aa, Homo sapiens
APELLGGP

SEQ ID NO: 152 - protein, 209 aa, Homo sapiens
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 102 aa, Homo sapiens
SVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 154 - protein, 19 aa, Homo sapiens
ERKCCVECPPCPAPPVAGP

SEQ ID NO: 155 - skipped

SEQ ID NO: 156 - protein, 9 aa, Homo sapiens
CCVECPPCP

SEQ ID NO: 157 - protein, 7 aa, Homo sapiens
APPVAGP

SEQ ID NO: 158 - protein, 209 aa, Homo sapiens
SVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 159 - protein, 102 aa, Homo sapiens
SVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 160 - protein, 70 aa, Homo sapiens
ELKTPLGDTTHTCPRCPEPKSCDTPPPCPRCPEPKSCDTPPPCPRCPEPKSCDTPPPCPRCPAPELLGGP

SEQ ID NO: 161 - protein, 12 aa, Homo sapiens
ELKTPLGDTTHT

SEQ ID NO: 162 - protein, 50 aa, Homo sapiens
CPRCPEPKSCDTPPPCPRCPEPKSCDTPPPCPRCPEPKSCDTPPPCPRCP

SEQ ID NO: 163 - protein, 8 aa, Homo sapiens
APELLGGP

SEQ ID NO: 164 - protein, 208 aa, Homo sapiens
SVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 165 - protein, 102 aa, Homo sapiens
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 166 - protein, 20 aa, Homo sapiens
ESKYGPPCPSCPAPEFLGGP

SEQ ID NO: 167 - protein, 12 aa, Homo sapiens
ESKYGPPCPPCP

SEQ ID NO: 168 - protein, 20 aa, Homo sapiens
ESKYGPPCPPCPAPEFLGGP

SEQ ID NO: 169 - protein, 7 aa, Homo sapiens
ESKYGPP

SEQ ID NO: 170 - protein, 5 aa, Homo sapiens
CPSCP

SEQ ID NO: 171 - protein, 8 aa, Homo sapiens
APEFLGGP

SEQ ID NO: 172 - protein, 209 aa, Homo sapiens
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 173 - protein, 18 aa, Homo sapiens
PSTPPTPSPSTPPTPSPS

SEQ ID NO: 174 - protein, 13 aa, Homo sapiens
VPCRVPPPPPCCH

SEQ ID NO: 175 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 176 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 177 - protein, 221 aa, synthetic construct
CKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 178 - protein, 221 aa, synthetic construct
CKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSELNVQKSNWEAGNTFTCSVLHEGLHNHHTEDSLSHSPGK

SEQ ID NO: 179 - protein, 360 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 180 - protein, 346 aa, synthetic construct
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNTGSGGGGSGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 181 - protein, 346 aa, synthetic construct
CDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGSGGGGSGTCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 182 - protein, 344 aa, synthetic construct
APGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPFTGSGGGGSGTCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSELNVQKSNWEAGNTFTCSVLHEGLHNHHTEDSLSHSPGK

SEQ ID NO: 183 - protein, 359 aa, synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGSGGGGSGTCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 184 - protein, 345 aa, synthetic construct
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNTGSGGGGSGTCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSELNVQKSNWEAGNTFTCSVLHEGLHNHHTEDSLSHSPGK

SEQ ID NO: 185 - protein, 397 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNTGSGGGGSGTCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSELNVQKSNWEAGNTFTCSVLHEGLHNHHTEDSLSHSPGK

SEQ ID NO: 186 - protein, 400 aa, synthetic construct
KSKAVEQVKNAFNKLQEKGIYKAMSEFDIFINYIEAYMTMKIRNGSGGGGSGTSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENTGSGGGGSGTCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSELNVQKSNWEAGNTFTCSVLHEGLHNHHTEDSLSHSPGK

SEQ ID NO: 187 - protein, 10 aa, synthetic construct
TGSGGGGSGT

SEQ ID NO: 188 - protein, 19 aa, synthetic construct
MGWSCIILFLVATATGVHS

SEQ ID NO: 189 - protein, 21 aa, synthetic construct
MACPGFLWALVISTCLEFSMA

SEQ ID NO: 190 - protein, 116 aa, Mus sp.
CDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 191 - protein, 113 aa, Mus sp.
APGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 192 - protein, 76 aa, synthetic construct [sequence omitted]

SEQ ID NO: 193 - protein, 77 aa, synthetic construct
MQIFVRTLTDRTITLEVEPSDTIENVRARIQDREGIPPDQQRLIFAGRQLEDGRTLSDYNIQKESTLHLVLRPRVVD

SEQ ID NO: 194 - protein, 398 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNTGSGGGGSGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 195 - protein, 350 aa, synthetic construct
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGSGGGGSGTCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 196 - protein, 120 aa, Mus sp.
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 197 - protein, 247 aa, synthetic construct
HHHHHHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGSGGGGSGTAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 198 - protein, 10 aa, synthetic construct
GGGGSGGGGS

SEQ ID NO: 199 - protein, 25 aa, synthetic construct
GGGGSGGGGSGGGGSGGGGSGGGGS

SEQ ID NO: 200 - protein, 30 aa, synthetic construct
GGGGSGGGGSGGGGSGGGGSGGGGSGGGGS

SEQ ID NO: 201 - protein, 35 aa, synthetic construct
GGGGSGGGGSGGGGSGGGGSGGGGSGGGGSGGGGS

SEQ ID NO: 202 - protein, 40 aa, synthetic construct
GGGGSGGGGSGGGGSGGGGSGGGGSGGGGSGGGGSGGGGS

SEQ ID NO: 203 - protein, 45 aa, synthetic construct
GGGGSGGGGSGGGGSGGGGSGGGGSGGGGSGGGGSGGGGSGGGGS

(19) United States
(12) Patent Application Publication
(10) Pub. No.: US 2026/0132178 A1
(43) Pub. Date: May 14, 2026

(54) ENGINEERED IMMUNOCYTOKINES, FUSION POLYPEPTIDES, AND IL10 POLYPEPTIDES

(71) Applicant: Synerkine Pharma B.V., Leiden (NL)

(72) Inventor: Theodorus Dinklo, Leiden (NL)

(21) Appl. No.: 19/196,406

(22) Filed: May 1, 2025

Related U.S. Application Data
(63) Continuation of application No. PCT/EP2023/080473, filed on Nov. 1, 2023.
(60) Provisional application No. 63/382,052, filed on Nov. 2, 2022.
(60) Provisional application No. 63/384,570, filed on Nov. 21, 2022.

(51) Int. Cl. C07K 14/54; A61K 9/00; A61K 38/00; A61P 29/00

(52) U.S. Cl. CPC C07K 14/5406; A61K 9/0019; A61K 38/00; A61P 29/00; C07K 14/5428; C07K 14/5437; C07K 2317/524; C07K 2317/526; C07K 2317/71; C07K 2319/35

(57) ABSTRACT

Provided are engineered heterodimeric immunocytokines, including fusions of IL4 and IL10, IL4 and IL13, or IL10 and IL13 to the heavy chains of an Fc fragment. In addition, fusion polypeptides are provided that comprise an engineered monomeric IL10 and a second cytokine. Also provided are engineered monomeric IL10 polypeptides with rearranged helices, for example, with an A helix joined to an F helix, in which a C-terminus of the D helix is not joined to an N-terminus of the E helix.

### CROSS REFERENCE

This application is a continuation of International Application No.: PCT/EP2023/080473 filed Nov. 1, 2023, which claims priority to and the benefit of United States Provisional Patent Application Nos. 63/382,052, filed Nov. 2, 2022, and 63/384,570, filed Nov. 21, 2022, each of which is incorporated herein by reference in its entirety.

### SEQUENCE LISTING

The instant application contains a Sequence Listing which has been submitted electronically in XML format and is hereby incorporated by reference by its entirety. Said XML copy, created May 1, 2025, is named 56780_708_301_SL.xml and is 204,920 bytes in size.

### SUMMARY

Disclosed herein, in some aspects, is an engineered immunocytokine comprising: (a) a first polypeptide chain comprising a first immunoglobulin heavy chain constant domain and IL4, wherein a C-terminus of the IL4 is joined to an N-terminus of the first immunoglobulin heavy chain constant domain via a first linker; and (b) a second polypeptide chain comprising a second immunoglobulin heavy chain constant domain and a second cytokine, wherein the second cytokine is IL10 or IL13, wherein a C-terminus of the second cytokine is joined to an N-terminus of the second immunoglobulin heavy chain constant domain via a second linker.

In some embodiments, the second cytokine is the IL10. In some embodiments, the IL10 is an engineered IL10 polypeptide comprising, from N-to-C terminus, an E helix, an F helix, an A helix, a B helix, a C helix, and a D helix. In some embodiments, the engineered IL10 polypeptide is engineered to reduce or substantially eliminate formation of an IL10 homodimer. In some embodiments, the engineered IL10 polypeptide comprises an amino acid sequence with at least 90% sequence identity to any one of SEQ ID NOs: 3-6. In some embodiments, the engineered IL10 polypeptide comprises an amino acid sequence with at least 95% sequence identity to any one of SEQ ID NOs: 3-6. In some embodiments, the engineered IL10 polypeptide comprises the amino acid sequence of any one of SEQ ID NOs: 3-6. In some embodiments, the engineered IL10 polypeptide consists essentially of the amino acid sequence of any one of SEQ ID NOs: 3-6. In some embodiments, the first polypeptide chain comprises an amino acid sequence with at least 90% sequence identity to any one of SEQ ID Nos: 108, 113, and 179. In some embodiments, the first polypeptide chain comprises the amino acid sequence of any one of SEQ ID Nos: 108, 113, and 179. In some embodiments, the second cytokine is the IL10 and the second polypeptide chain comprises an amino acid sequence with at least 90% sequence identity to SEQ ID NO: 110 or 116. In some embodiments, the second cytokine is the IL10 and the second polypeptide chain comprises the amino acid sequence of SEQ ID NO: 110 or 116. In some embodiments, the second cytokine is the IL10 and the second polypeptide chain comprises an amino acid sequence with at least 90% sequence identity to any one of SEQ ID NOs: 111, 118, and 194. In some embodiments, the second cytokine is the IL10 and the second polypeptide chain comprises the amino acid sequence of any one of SEQ ID NOs: 111, 118, and 194. In some embodiments, the second cytokine is the IL13. In some embodiments, the second cytokine is the IL13 and the second polypeptide chain comprises an amino acid sequence with at least 90% sequence identity to any one of SEQ ID NOs: 109, 115, and 180. In some embodiments, the second cytokine is the IL13 and the second polypeptide chain comprises the amino acid sequence of any one of SEQ ID NOs: 109, 115, and 180. In some embodiments, the first immunoglobulin heavy chain constant domain and the second immunoglobulin heavy chain each comprise a CH2 domain and a CH3 domain. In some embodiments, the first polypeptide chain and the second polypeptide chain each comprise a heterodimerization domain.

Disclosed herein, in some aspects, is an engineered IL10 polypeptide comprising an A helix, an F helix, and a linker, wherein the A helix is joined to the F helix via the linker, and the engineered IL10 polypeptide is in a monomeric form.

Disclosed herein, in some aspects, is an engineered IL10 polypeptide comprising a D helix and an E helix, wherein a C-terminus of the D helix is not joined to an N-terminus of the E helix.

Disclosed herein, in some aspects, is an engineered IL10 polypeptide comprising, from N-to-C terminus, an E helix, an F helix, an A helix, a B helix, a C helix, and a D helix, wherein the engineered IL10 polypeptide is engineered to reduce or substantially eliminate formation of an IL10 homodimer.

In some embodiments, the engineered IL10 polypeptide, further comprises a linker. In some embodiments, an N-terminus of the A helix is joined to a C-terminus of the F helix via the linker. In some embodiments, the linker comprises the amino acid sequence of any one of SEQ ID NOs: 22-64. In some embodiments, the engineered IL10 polypeptide does not contain a second A helix or a second F helix. In some embodiments, the engineered IL10 polypeptide does not contain a second D helix or a second E helix. In some embodiments, the engineered IL10 polypeptide does not contain a second B helix or a second C helix. In some embodiments, the engineered IL10 polypeptide is engineered to reduce or substantially eliminate formation of an IL10 homodimer. In some embodiments, the engineered IL10 polypeptide comprises an amino acid sequence with at least 90% sequence identity to any one of SEQ ID NOs: 3-6. In some embodiments, the engineered IL10 polypeptide comprises an amino acid sequence with at least 95% sequence identity to any one of SEQ ID NOs: 3-6. In some embodiments, the engineered IL10 polypeptide comprises the amino acid sequence of any one of SEQ ID NOs: 3-6. In some embodiments, the engineered IL10 polypeptide consists essentially of the amino acid sequence of any one of SEQ ID NOs: 3-6. In some embodiments, the helices of the engineered IL10 are helices of mammalian IL10 or variant thereof. In some embodiments, the helices of the engineered IL10 are helices of human IL10 or variant thereof.

Disclosed herein, in some aspects, is a fusion polypeptide comprising an immunoglobulin constant domain and the engineered IL10 polypeptide of any one of the preceding embodiments.

In some embodiments, the immunoglobulin constant domain comprises a heavy chain constant domain. In some embodiments, the heavy chain constant domain comprises a mammalian heavy chain constant domain. In some embodiments, the heavy chain constant domain comprises a human heavy chain constant domain. In some embodiments, the heavy chain constant domain comprises a human IgG1 CH2 domain and CH3 domain. In some embodiments, the heavy chain constant domain comprises a human IgG4 CH2 domain and CH3 domain. In some embodiments, the heavy chain constant domain comprises a human IgA CH2 domain and CH3 domain. In some embodiments, the immunoglobulin constant domain comprises a light chain constant domain. In some embodiments, the fusion polypeptide comprises an amino acid sequence with at least 86% sequence identity to SEQ ID NO: 110 or SEQ ID NO: 116. In some embodiments, the fusion polypeptide comprises an amino acid sequence with at least 90% sequence identity to SEQ ID NO: 110 or SEQ ID NO: 116. In some embodiments, the fusion polypeptide comprises an amino acid sequence with at least 95% sequence identity to SEQ ID NO: 110 or SEQ ID NO: 116. In some embodiments, the fusion polypeptide comprises the amino acid sequence of SEQ ID NO: 110 or SEQ ID NO: 116.

Disclosed herein, in some aspects, is a fusion polypeptide comprising the engineered IL10 polypeptide of any one of the preceding embodiments and a second cytokine.

In some embodiments, the second cytokine comprises an interleukin 4 (IL4) polypeptide. In some embodiments, the IL4 polypeptide comprises an amino acid sequence with at least 90% sequence identity to any one of SEQ ID NOs: 10-13. In some embodiments, the IL4 polypeptide comprises an amino acid sequence with at least 95% sequence identity to any one of SEQ ID NOs: 10-13. In some embodiments, the IL4 polypeptide comprises the amino acid sequence of any one of SEQ ID NOs: 10-13. In some embodiments, the fusion polypeptide comprises an amino acid sequence with at least 65% sequence identity to SEQ ID NO: 65. In some embodiments, the fusion polypeptide comprises an amino acid sequence with at least 80% sequence identity to SEQ ID NO: 65. In some embodiments, the fusion polypeptide comprises an amino acid sequence with at least 90% sequence identity to SEQ ID NO: 65. In some embodiments, the fusion polypeptide comprises the amino acid sequence of SEQ ID NO: 65. In some embodiments, the second cytokine comprises an interleukin 13 (IL13) polypeptide. In some embodiments, the IL13 polypeptide comprises an amino acid sequence with at least 90% sequence identity to any one of SEQ ID NOs: 14-21. In some embodiments, the IL13 polypeptide comprises an amino acid sequence with at least 95% sequence identity to any one of SEQ ID NOs: 14-21. In some embodiments, the IL13 polypeptide comprises the amino acid sequence of any one of SEQ ID NOs: 14-21. In some embodiments, the fusion polypeptide comprises an amino acid sequence with at least 80% sequence identity to any one of SEQ ID NOs: 71-74.

Disclosed herein, in some aspects, is an engineered immunocytokine comprising: (a) a first polypeptide chain comprising a first immunoglobulin heavy chain constant domain and a first cytokine, wherein the first cytokine is an IL4 polypeptide, IL10 polypeptide, or IL13 polypeptide; and (b) a second polypeptide chain comprising a second immunoglobulin heavy chain constant domain and a second cytokine, wherein the first cytokine and the second cytokine are different.

In some embodiments, the first cytokine is the IL4 polypeptide. In some embodiments, the first cytokine is the IL10 polypeptide. In some embodiments, the first cytokine is the IL13 polypeptide. In some embodiments, the first cytokine is the IL4 polypeptide and the second cytokine is the IL10 polypeptide. In some embodiments, the first cytokine is the IL4 polypeptide and the second cytokine is the IL13 polypeptide. In some embodiments, the first cytokine is the IL10 polypeptide and the second cytokine is the IL13 polypeptide. In some embodiments, the first cytokine is a wild type cytokine. In some embodiments, the second cytokine is a wild type cytokine. In some embodiments, the first cytokine is a mammalian cytokine. In some embodiments, the first cytokine is human cytokine. In some embodiments, the second cytokine is a mammalian cytokine. In some embodiments, the second cytokine is a human cytokine. In some embodiments, the IL4 polypeptide comprises an amino acid sequence with at least about 90% sequence identity to any one of SEQ ID NOs: 10-13. In some embodiments, the IL10 polypeptide comprises an amino acid sequence with at least about 90% sequence identity to any one of SEQ ID NOs: 1-9. In some embodiments, the IL13 polypeptide comprises an amino acid sequence with at least about 90% sequence identity to any one of SEQ ID NOs: 14-21. In some embodiments, the first immunoglobulin heavy chain constant domain comprises a CH2 domain and a CH3 domain. In some embodiments, the second immunoglobulin heavy chain constant domain comprises a CH2 domain and a CH3 domain. In some embodiments, the first immunoglobulin heavy chain constant domain and the second immunoglobulin heavy chain constant domain each comprise comprises a human IgG CH2 domain and CH3 domain. In some embodiments, the engineered immunocytokine comprises an Fc region that comprises the first immunoglobulin heavy chain constant domain and the second immunoglobulin heavy chain constant domain. In some embodiments, the Fc region comprises a modification that reduces Fc receptor-mediated effector function. In some embodiments, the Fc region comprises a modification that reduces Fc receptor binding. In some embodiments, the first immunoglobulin heavy chain constant domain and the second immunoglobulin heavy chain constant domain form a covalently-linked heterodimer. In some embodiments, the first polypeptide chain and the second polypeptide chain each comprise a heterodimerization domain. In some embodiments, the engineered immunocytokine comprises a knob-in-hole interaction between the first immunoglobulin heavy chain constant domain and the second immunoglobulin heavy chain constant domain. In some embodiments, a C-terminus of the first cytokine is joined to an N-terminus of the first immunoglobulin heavy chain constant domain. In some embodiments, a C-terminus of the second cytokine is joined to an N-terminus of the second immunoglobulin heavy chain constant domain. In some embodiments, the C-terminus of the first cytokine is joined to the N-terminus of the first immunoglobulin heavy chain constant domain via a linker that comprises the amino acid sequence of any one of SEQ ID NOs: 22-64. In some embodiments, the C-terminus of the second cytokine is joined to the N-terminus of the second immunoglobulin heavy chain constant domain via a linker that comprises the amino acid sequence of any one of SEQ ID NOs: 22-64. In some embodiments, the first polypeptide chain comprises an amino acid sequence with at least 90% sequence identity to any one of SEQ ID NOs: 108-118, 179, 180, and 194. In some embodiments, the second polypeptide chain comprises an amino acid sequence with at least 90% sequence identity to any one of SEQ ID NOs: 108-118, 179, 180, and 194. In some embodiments, the first polypeptide chain comprises the amino acid sequence of any one of SEQ ID NOs: 108-118, 179, 180, and 194. In some embodiments, the second polypeptide chain comprises the amino acid sequence of any one of SEQ ID NOs: 108-118, 179, 180, and 194. In some embodiments, the engineered immunocytokine exhibits a plasma half-life of at least two hours after intravenous administration as determined by a rodent pharmacokinetic assay.

Disclosed herein, in some aspects, is an engineered immunocytokine comprising: (a) an IL4 receptor binding domain; (b) an IL10 receptor binding domain or an IL13 receptor binding domain; and (c) a stabilizing domain; wherein the engineered immunocytokine exhibits a plasma half-life of at least two hours after intravenous administration as determined by a rodent pharmacokinetic assay.

In some embodiments, the stabilizing domain comprises an immunoglobulin heavy chain constant domain. In some embodiments, the stabilizing domain comprises an immunoglobulin CH2 domain and CH3 domain. In some embodiments, the stabilizing domain comprises a human IgG CH2 domain and CH3 domain. In some embodiments, the stabilizing domain comprises a human IgG1 CH2 domain and CH3 domain. In some embodiments, the stabilizing domain comprises a human IgG4 CH2 domain and CH3 domain. In some embodiments, the stabilizing domain comprises a human IgA CH2 domain and CH3 domain. In some embodiments, the stabilizing domain comprises an immunoglobulin Fc domain. In some embodiments, the IL4 receptor binding domain comprises a mammalian IL4 or a receptor-binding fragment thereof. In some embodiments, the engineered immunocytokine comprises the IL10 receptor binding domain, wherein the IL10 receptor binding domain comprises a mammalian IL10 or a receptor-binding fragment thereof. In some embodiments, the engineered immunocytokine comprises the IL13 receptor binding domain, wherein the IL13 receptor binding domain comprises a mammalian IL13 or a receptor-binding fragment thereof. In some embodiments, the IL4 receptor binding domain activates IL4 receptor signaling, the IL10 receptor binding domain activates IL10 receptor signaling, and the IL13 receptor binding domain activates IL13 receptor signaling.

Disclosed herein, in some aspects, is an engineered immunocytokine comprising: (a) a first polypeptide chain comprising a first immunoglobulin heavy chain constant domain and a first cytokine; and (b) a second polypeptide chain comprising a second immunoglobulin heavy chain constant domain and a second cytokine, wherein the first cytokine and the second cytokine are different.

In some embodiments, a C-terminus of the first cytokine is joined to an N-terminus of the first immunoglobulin heavy chain constant domain, optionally via a linker, and a C-terminus of the second cytokine is joined to an N-terminus of the second immunoglobulin heavy chain constant domain, optionally via a linker. In some embodiments, an N-terminus of the first cytokine is joined to a C-terminus of the first immunoglobulin heavy chain constant domain, optionally via a linker, and an N-terminus of the second cytokine is joined to a C-terminus of the second immunoglobulin heavy chain constant domain, optionally via a linker.

Disclosed herein, in some aspects, is an engineered polypeptide comprising an IL4 polypeptide joined to a first immunoglobulin heavy chain, wherein the first immunoglobulin heavy chain is engineered to heterodimerize with a second immunoglobulin heavy chain.

In some embodiments, the first immunoglobulin heavy chain is engineered to heterodimerize with a second immunoglobulin heavy chain. In some embodiments, a C-terminus of the IL4 polypeptide is joined to an N-terminus of the first immunoglobulin heavy chain. In some embodiments, a C-terminus of the IL4 polypeptide is joined to an N-terminus of the first immunoglobulin heavy chain via a linker that comprises the amino acid sequence of any one of SEQ ID NOs: 22-64. In some embodiments, the engineered polypeptide comprises an amino acid sequence with at least 90% sequence identity to SEQ ID NO: 108, 112, or 113. In some embodiments, the engineered polypeptide comprises an amino acid sequence with at least 95% sequence identity to SEQ ID NO: 108, 112, or 113. In some embodiments, the engineered polypeptide comprises the amino acid sequence of SEQ ID NO: 108, 112, or 113.

Disclosed herein, in some aspects, is an engineered polypeptide comprising (a) an IL10 polypeptide joined to a first immunoglobulin heavy chain, wherein: the IL10 polypeptide is engineered to reduce or substantially eliminate formation of an IL10 homodimer; and (b) the first immunoglobulin heavy chain is engineered to heterodimerize with a second immunoglobulin heavy chain.

In some embodiments, the IL10 polypeptide comprises an amino acid sequence with at least 90% sequence identity to any one of SEQ ID NOs: 3-9. In some embodiments, the IL10 polypeptide consists essentially of an amino acid sequence with at least 90% sequence identity to any one of SEQ ID NOs: 3-9. In some embodiments, the first immunoglobulin heavy chain is engineered to heterodimerize with the second immunoglobulin heavy chain.

Disclosed herein, in some aspects, is an engineered polypeptide comprising an amino acid sequence with at least 65% sequence identity to SEQ ID NO: 65.

In some embodiments, the engineered polypeptide comprises an amino acid sequence with at least 90% sequence identity to SEQ ID NO: 65. In some embodiments, the engineered polypeptide comprises the amino acid sequence of SEQ ID NO: 65.

Disclosed herein, in some aspects, is an engineered polypeptide comprising an amino acid sequence with at least 96% sequence identity to SEQ ID NO: 108.

In some embodiments, the engineered polypeptide comprises the amino acid sequence of SEQ ID NO: 108 or 113.

Disclosed herein, in some aspects, is an engineered polypeptide comprising an amino acid sequence with at least 98% sequence identity to SEQ ID NO: 109.

In some embodiments, the engineered polypeptide comprises the amino acid sequence of SEQ ID NO: 109 or 115.

Disclosed herein, in some aspects, is an engineered polypeptide comprising an amino acid sequence with at least 86% sequence identity to SEQ ID NO: 110 or 116.

In some embodiments, the engineered polypeptide can amino acid sequence with at least 90% sequence identity to SEQ ID NO: 110 or 116. In some embodiments, the engineered polypeptide can amino acid sequence with at least 95% sequence identity to SEQ ID NO: 110 or 116. In some embodiments, the engineered polypeptide comprises the amino acid sequence of SEQ ID NO: 110 or 116.

Disclosed herein, in some aspects, is an engineered polypeptide comprising an amino acid sequence with at least 98% sequence identity to SEQ ID NO: 111.

In some embodiments, the engineered polypeptide comprises the amino acid sequence of SEQ ID NO: 111. In some embodiments, the engineered polypeptide comprises an Fc region with a modification that reduces Fc receptor-mediated effector function.

Disclosed herein, in some aspects, is a nucleic acid encoding the engineered IL10 polypeptide, the fusion polypeptide, the engineered immunocytokine, or the engineered polypeptide of any one of the preceding embodiments. Disclosed herein, in some aspects, is a vector comprising the nucleic acid.

Disclosed herein, in some aspects, is a pharmaceutical composition comprising the engineered IL10 polypeptide of any one of the preceding embodiments and a pharmaceutically-acceptable excipient, wherein at least 70% of the engineered IL10 polypeptide is in a monomeric form.

Disclosed herein, in some aspects, is a pharmaceutical composition comprising the engineered IL10 polypeptide of any one of the preceding embodiments and a pharmaceutically-acceptable excipient, wherein no more than about 30% of the engineered IL10 polypeptide is in a dimeric form.

Disclosed herein, in some aspects, is a pharmaceutical composition comprising the fusion polypeptide of any one of the preceding embodiments and a pharmaceutically-acceptable excipient, wherein at least 70% of the fusion polypeptide is in a monomeric form.

Disclosed herein, in some aspects, is a pharmaceutical composition comprising the fusion polypeptide of any one of the preceding embodiments and a pharmaceutically-acceptable excipient, wherein no more than about 30% of the fusion polypeptide is in a dimeric form.

Disclosed herein, in some aspects, is a pharmaceutical composition comprising the fusion polypeptide of any one of the preceding embodiments and a pharmaceutically-acceptable excipient, wherein at most 30% of the engineered IL10 polypeptide is in an IL10 homodimeric form.

Disclosed herein, in some aspects, is a pharmaceutical composition comprising a pharmaceutically-acceptable excipient and the engineered IL10 polypeptide, the fusion polypeptide, the engineered immunocytokine, the engineered polypeptide, the nucleic acid, or the vector of any one of the preceding embodiments.

Disclosed herein, in some aspects, is a method of treating a condition in a subject in need thereof, the method comprising administering to the subject an effective amount of the engineered IL10 polypeptide, the fusion polypeptide, the engineered immunocytokine, the engineered polypeptide, the nucleic acid, the vector, or the pharmaceutical composition of any one of the preceding embodiments.

In some embodiments, the condition comprises pain. In some embodiments, the condition comprises chronic pain. In some embodiments, the condition comprises neuropathic pain. In some embodiments, the condition comprises inflammatory pain. In some embodiments, the condition comprises inflammation. In some embodiments, the condition comprises chronic inflammation. In some embodiments, the condition is osteoarthritis. In some embodiments, the condition comprises a neuropathy. In some embodiments, the condition comprises a chemotherapy-induced neuropathy. In some embodiments, the condition comprises neurodegeneration. In some embodiments, the administering comprises systemic administration. In some embodiments, the administering comprises intravenous administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows an illustrative structure of an engineered IL10 polypeptide comprising, from N-to-C terminus, helices E, F, A, B, C, and D, with a linker joining helices A and F.

FIG. 1B shows an illustrative structure of an engineered IL10 polypeptide comprising, from N-to-C terminus, helices A-F, with an insertion between helices D and E.

FIG. 2 provides an illustrative schematic of a heterodimeric immunocytokine with IL4 appended to the N-terminus of a first heavy chain of an Fc fragment and an engineered IL10 appended to the N-terminus of the second heavy chain, with a knob-in-hole modification to induce heterodimerization of the chains.

FIG. 3 is a NuPage protein gel showing fusion polypeptides under non-reduced (NR) and reduced (R) conditions. Lanes 2-5 show results for non-reducing conditions, lanes 7-10 show results for reducing conditions. Lanes 2, 3, 7, and 8 are an engineered IL4-IL10 immunocytokine with SEQ ID NO: 108 paired with SEQ ID NO: 111 (comprising an engineered IL10 with an insertion between helices D and E). Lanes 4, 5, 9, and 10 are an engineered IL4-IL10 immunocytokine with SEQ ID NO: 108 paired with SEQ ID NO: 110 (comprising an engineered IL10 with rearranged helices as disclosed herein).

FIG. 4A shows bioluminescence as a function of time for an enzyme complementation bioluminescence-based protein-protein interaction assay, reflecting proximity of IL4 receptor and IL10 receptor upon treatment. Results are shown for (i) unfused IL4+IL10, (ii) a control fusion protein comprising wild type IL4 joined to IL10 (IL4-IL10, 0.088 nM), (iii) an engineered heterodimeric immunocytokine comprising IL4 and a monomeric form of IL10 with an insertion between helices D and E (IL4-IL10 Fc (J), 0.35 nM), and (iv) an engineered heterodimeric immunocytokine comprising IL4 and a monomeric form of IL10 with rearranged helices as disclosed herein (IL4-IL10 Fc (S), 0.35 nM). The unfused IL4+IL10 were added at concentration equivalent to a supramaximal concentration of IL4-IL10.

FIG. 4B shows bioluminescence as a function of time for an enzyme complementation bioluminescence-based protein-protein interaction assay, reflecting proximity of IL4 receptor and IL10 receptor upon treatment. Results are shown for (i) unfused IL4+IL10, (ii) a control fusion protein comprising wild type IL4 joined to IL10 (IL4-IL10, 3 nM), (iii) an engineered heterodimeric immunocytokine comprising IL4 and a monomeric form of IL10 with an insertion between helices D and E (IL4-IL10 Fc (J), 12 nM), and (iv) an engineered heterodimeric immunocytokine comprising IL4 and a monomeric form of IL10 with rearranged helices as disclosed herein (IL4-IL10 Fc (S), 12 nM). The unfused IL4+IL10 were added at concentration equivalent to a supramaximal concentration of IL4-IL10.

FIG. 5A shows bioluminescence for an enzyme complementation bioluminescence-based protein-protein interaction assay, reflecting proximity of IL4Ra and IL13Ra2 upon treatment. Results are shown for (i) unfused IL4+IL13, (ii) a control fusion protein comprising IL4 joined to IL13 (IL4-IL13), and (iii) an engineered heterodimeric immunocytokine comprising IL4 and IL13 (IL4-IL13 Fc).

FIG. 5B shows bioluminescence as a function of time for an enzyme complementation bioluminescence-based protein-protein interaction assay, reflecting proximity of IL4Ra and IL13Ra2 upon treatment. Results are shown for (i) unfused IL4+IL13, (ii) a control fusion protein comprising IL4 joined to IL13 (IL4-IL13), and (iii) an engineered heterodimeric immunocytokine comprising IL4 and IL13 (IL4-IL13 Fc). Treatments were at 30 nM (IL4-IL13 and IL4-IL13 Fc) or an equimolar equivalent (IL4+IL13).

### DETAILED DESCRIPTION

Cytokines have potential as therapeutics due to their ability to powerfully modulate a wide variety of physiological and pathological processes via endogenous signal transduction pathways, including processes associated with inflammation, pain, and neuropathy. For example, regulatory cytokines can reduce inflammation, suppress sensitization and dysfunction of sensory neurons, and reduce activation of pain pathways. However, the efficacy of regulatory cytokines has been limited due to a short duration of action, poor bioavailability, and toxicity associated with broad elicitation of signaling on non-target cells.

To harness the potential of cytokines clinically, there is a need to develop therapeutics with improved half-life in a subject and an ability to elicit therapeutic effects via engaging multiple target receptors synergistically.

Compositions and methods disclosed herein can comprise engineered immunocytokines, including heterodimeric immunocytokines comprising synergistic combinations of cytokines fused to the chains of immunoglobulin heavy chains. Engineered immunocytokines disclosed herein can have advantages over the parent cytokines (e.g., over corresponding individual cytokines, or over the cytokines present in alternate constructs, such as fusion proteins). These advantages can include, for example, a simpler dosing regimen, longer half-life within a subject, and an ability to bind target receptors in close spatial and temporal proximity to elicit a desirable signaling profile. In some embodiments, the ability of an engineered heterodimeric immunocytokine disclosed herein to preferentially induce signaling in cells co-expressing a pair of cytokine receptors rather than just one (e.g., IL4 receptor and IL10 receptor, IL4 receptor and IL13 receptor, or IL10 receptor and IL13 receptor) can result in an advantageous efficacy and/or toxicity profile in vivo. In some embodiments, provided engineered heterodimeric immunocytokines provide superior neuro-protective, anti-inflammatory, and/or analgesic properties compared to unfused cytokines and alternative constructs.

Also provided herein are engineered IL10 polypeptides that exhibit reduced formation of IL10 dimers, enhanced retention of IL10 in a monomeric form, and/or improved functionality of the IL10 (e.g., induction of IL10 receptor signaling) when the C-terminus of the engineered IL10 is joined to the N-terminus of a fusion partner (for example, joined to one arm of an Fc fragment, optionally via a linker, or joined to another cytokine, such as IL4 or IL13).

### Cytokines

Compositions and methods disclosed herein can comprise one or more cytokines, for example, IL10, IL4, IL13, IL27, IL27A, IL33, TGFβ1, TGFβ2, a variant thereof, or a functional fragment thereof.

When describing a cytokine, the term “wild type” can describe a cytokine with an amino acid sequence that is naturally occurring and encoded by a germline genome of a given species. A species can have one wild type sequence, or two or more wild type sequences (for example, with one canonical wild type sequence and one or more non-canonical wild type sequences). A wild type cytokine sequence can include a sequence that is truncated at the N and/or C terminus relative to the sequence encoded by an open reading frame. A wild type cytokine sequence can be a mature form of a cytokine that has been processed to remove N-terminal and/or C-terminal residues. A wild type cytokine can lack a signal peptide or can include a signal peptide (e.g., a signal peptide can be added to the N-terminus of the wild type cytokine).

When describing a cytokine, the term “derivative” can describe a cytokine with an amino acid sequence that differs from a wild type sequence by one or more amino acids, for example, containing one or more amino acid insertions, deletions, or substitutions relative to a wild type sequence. A cytokine derivative binds to at least one subunit of the corresponding native receptor for the wild type cytokine and elicits signaling and/or cytokine activity. The binding affinity, signaling, and/or cytokine activity of a cytokine derivative can be the same or different than the corresponding wild type cytokine.

“Functional”, in relation to the cytokines or variants or fragments thereof can describe the capability to display cytokine functionality, for example, the ability to bind to at least one receptor subunit that a wild type version of the cytokine binds, elicit signaling, and/or generate a readout in a functional assay.

### A. Interleukin 10 (IL10)

Compositions and methods disclosed herein can comprise an interleukin 10 (IL10) polypeptide. An IL10 polypeptide can be a mammalian or a human IL10 polypeptide, a variant thereof (for example, an engineered IL10 polypeptide described herein), or a functional fragment thereof. In some embodiments, an IL10 polypeptide is a human IL10 polypeptide, or a variant or functional fragment thereof. In some embodiments, an IL10 polypeptide is a murine, rodent, canine, feline, equine, porcine, primate, or bovine IL10 polypeptide, or a variant or functional fragment thereof. In some embodiments, an IL10 polypeptide is a non-human and/or non-rodent mammalian IL10 polypeptide, or a variant or functional fragment thereof.

Human and mouse IL can each be about 178-184 amino acids in length in their immature form (including a signal sequence), and about 160 amino acids in their mature form (e.g., after removal of the signal sequence). Human and mouse IL10 can comprise six alpha helices, helix A, helix B, helix C, helix D, helix E, and helix F. Helices A to D of one monomer of wild type IL10 can noncovalently interact with helices E and F of a second monomer, forming a noncovalent V-shaped homodimer.

An IL10 polypeptide of the disclosure can comprise a wild type IL10 sequence. A non-limiting example of a wild type IL sequences is SEQ ID NO: 1. SEQ ID NO: 1 can be a canonical wild type IL10 sequence of the disclosure. The A-F helices can correspond to, e.g., SEQ ID NOs: 140-145, respectively.

TABLE 1 provides illustrative sequences of IL10 and the A to F helices of IL10

TABLE 1

| SEQ | | |
|---|---|---|
| ID | | |
| NO: | Description | Sequence |
| 1 | Mature | SPGQGTQSENSCTHFPGNLPNMLRDLRDAFSRVKTFFQMKDQ |
| | human IL10 | LDNLLLKESLLEDFKGYLGCQALSEMIQFYLEEVMPQAENQDP |
| | | DIKAHVNSLGENLKTLRLRLRRCHRFLPCENKSKAVEQVKNAF |
| | | NKLQEKGIYKAMSEFDIFINYIEAYMTMKIRN |
| 2 | Human IL10 | MHSSALLCCLVLLTGVRASPGQGTQSENSCTHFPGNLPNMLRD |
| | with signal | LRDAFSRVKTFFQMKDQLDNLLLKESLLEDFKGYLGCQALSE |
| | sequence | MIQFYLEEVMPQAENQDPDIKAHVNSLGENLKTLRLRLRRCHR |
| | | FLPCENKSKAVEQVKNAFNKLQEKGIYKAMSEFDIFINYIEAY |
| | | MTMKIRN |
| 140 | A helix of | NLPNMLRDLRDAFSRVKTFFQMKD |
| | human IL10 | |
| 141 | B helix of | KESLLEDFKG |
| | human IL10 | |
| 142 | C helix of | LGCQALSEMIQFYLEEVMPQAEN |
| | human IL10 | |
| 143 | D helix of | IKAHVNSLGENLKTLRLRLRRC |
| | human IL10 | |
| 144 | E helix of | SKAVEQVKNAFNKL |
| | human IL10 | |
| 145 | F helix of | EKGIYKAMSEFDIFINYIEAYMTMKIR |
| | human IL10 | |

An IL10 polypeptide of the disclosure can comprise an IL10 variant, derivative, or fragment thereof with one or more amino acid substitutions. For example, an IL10 variant, derivative, or fragment thereof can comprise an amino acid substitution at position N18, D55, I69, I87, A89, N92, N97, K99, H109, R110, F111, N148, Y153, M156, or a combination thereof relative to SEQ ID NO: 1. In some embodiments, an IL10 variant, derivative, or fragment thereof comprises a substitution that is N18Y, N18I, N92I, K99N, F111S, I87A, I87G, A89D, H109D, R110D, Y153D, M156D, A89D, H109E, R110E, Y153E, M156E, or a combination thereof relative to SEQ ID NO: 1. In some embodiments, an IL10 variant, derivative, or fragment thereof comprises a substitution that is N18Y, N18I, N92I, K99N, or a combination thereof relative to SEQ ID NO: 1.

In some embodiments, an IL10 variant, derivative, or fragment thereof does not contain a substitution at position 187, A89, H109, R110, F111, Y153, or M156 relative to SEQ ID NO: 1. In some embodiments, an IL10 variant, derivative, or fragment thereof does not contain a substitution at position N18, N92, or K99 relative to SEQ ID NO: 1. In some embodiments, an IL10 variant, derivative, or fragment thereof does not contain a F111S, I87A, I87G, A89D, H109D, R110D, Y153D, M156D, A89D, H109E, R110E, Y153E, or M156E substitution. In some embodiments, an IL10 variant, derivative, or fragment thereof does not contain a N18Y, N18I, N92I, or K99N substitution.

An IL10, variant, derivative, or fragment thereof (which can include an engineered IL10 polypeptide) can bind to an IL10 receptor. An IL10 receptor can comprise, for example, interleukin 10 receptor 1 (IL-10R1) and interleukin 10 receptor 2 (IL-10R2). In some embodiments, an IL10 or IL10 variant, derivative, or fragment thereof of the disclosure can activate a native IL10 receptor. A native IL10 receptor can be, for example, a receptor comprising an IL-10R1 subunit and an IL-10R2 subunit. In some embodiments, an IL10 or IL10 variant, derivative, or fragment thereof of the disclosure can activate a native IL10 receptor when present in a fusion polypeptide disclosed herein, for example, fused to IL4 via a linker, fused to IL13 via a linker, or fused to an immunoglobulin constant domain (e.g., fused to an Fc domain on one chain of a heterodimeric immunocytokine).

An IL10 polypeptide, variant, derivative, or fragment thereof (which can include an engineered IL10 polypeptide) can bind to one or more IL10 receptor subunits, for example, IL-10R1, IL-10R2, or a combination thereof. In some embodiments an IL10 polypeptide binds to IL-10R1 and IL-10R2. In some embodiments an IL10 polypeptide binds to IL-10R1. In some embodiments an IL10 polypeptide binds to IL-10R2.

In some embodiments, an IL10 polypeptide or IL10 variant, derivative, or fragment thereof of the disclosure binds to an IL10 receptor subunit with about a comparable affinity as a wild type IL10 sequence. A comparable affinity can be, for example, less than about 50, less than about 25, less than about 10, less than about 5, less than about 2, less than about 1.5, or less than about 1.1 fold increased affinity compared to a wild type IL10 sequence. A comparable affinity can be, for example, less than about 50, less than about 25, less than about 10, less than about 5, less than about 2, less than about 1.5, or less than about 1.1 fold decreased affinity compared to a wild type IL10 sequence. In some embodiments, an IL10 or IL10 variant, derivative, or fragment thereof of the disclosure can bind to an IL-10R1 and an IL-10R2 with at least a comparable affinity as a wild type IL10. In some embodiments, an IL10 or IL10 variant, derivative, or fragment thereof of the disclosure can bind to an IL10 receptor subunit with at most a comparable affinity as a wild type IL10.

In some embodiments, an IL10 or IL10 variant, derivative, or fragment thereof can bind to an IL10 receptor or IL10 receptor subunit with at least 1.5 fold, at least 2 fold, at least 5 fold, at least 10 fold, at least 20 fold, at least 50 fold, at least 100 fold, at least 500 fold, at least 1000 fold, or at least 10,000 fold increased affinity relative to a wild type IL10 sequence. In some embodiments, an IL10 or IL10 variant, derivative, or fragment thereof can bind to an IL10 receptor or IL10 receptor subunit with at least 1.5 fold, at least 2 fold, at least 5 fold, at least 10 fold, at least 20 fold, at least 50 fold, at least 100 fold, at least 500 fold, at least 1000 fold, or at least 10,000 fold decreased affinity relative to a wild type IL10 sequence.

In some embodiments, a polypeptide of the disclosure does not contain IL10.

### 1. Engineered IL10 Polypeptide

An IL10 polypeptide used in compositions and methods of the disclosure can be an engineered IL10 polypeptide. An IL10 polypeptide can be engineered, for example, to reduce formation of IL10 dimers, promote retention of IL10 in a monomeric form, and/or facilitate functionality of the IL10 (e.g., induction of IL10 receptor signaling) when the C-terminus of the engineered IL10 is joined to the N-terminus of a fusion partner (for example, joined to one arm of an Fc fragment, optionally via a linker, or joined to another cytokine, such as IL4 or IL13). In some embodiments, an engineered IL10 is engineered to reduce or substantially eliminate formation of an IL10 homodimer.

An engineered IL10 polypeptide can comprise one or more helices of a mammalian IL10, such as human IL10. The helices can be arranged, oriented, or joined differently to wild type IL10, which contains helices A-F from N-to-C terminus.

An engineered IL10 polypeptide can comprise an A helix joined to an F helix via a linker. For example, the C-terminus of the F helix can be joined to the N-terminus of the A helix via a linker peptide disclosed herein (e.g., as compared to a wild type IL10, in which the N-terminus of the A-helix is at the N-terminus of the protein, and the C-terminus of the F helix is at the C-terminus of the protein). In some embodiments, the engineered IL10 polypeptide does not contain a second A helix. In some embodiments, the engineered IL10 polypeptide does not contain a second F helix. In some embodiments, the engineered IL10 polypeptide does not contain a second A helix or a second F helix.

In some embodiments, the engineered IL10 polypeptide comprises an A helix, a B helix, a C helix, a D helix, an E helix, and an F helix, wherein the A helix is joined to the F helix via a linker. In some embodiments, the engineered IL10 polypeptide does not contain a second A helix, B helix, C helix, D helix, E helix, and/or F helix.

An engineered IL10 polypeptide can comprise a D helix and an E helix, wherein the C-terminus of the D helix is not joined to the N-terminus of the E helix (e.g., as compared to wild type IL10, in which the D and E helices are adjacent to one another). In some embodiments, the engineered IL10 polypeptide does not contain a second D helix. In some embodiments, the engineered IL10 polypeptide does not contain a second E helix. In some embodiments, the engineered IL10 polypeptide does not contain a second D helix or a second E helix.

In some embodiments, the engineered IL10 polypeptide comprises an A helix, a B helix, a C helix, a D helix, an E helix, and an F helix, wherein the C-terminus of the D helix is not joined to the N-terminus of the E helix. In some embodiments, the engineered IL10 polypeptide does not contain a second A helix, B helix, C helix, D helix, E helix, and/or F helix.

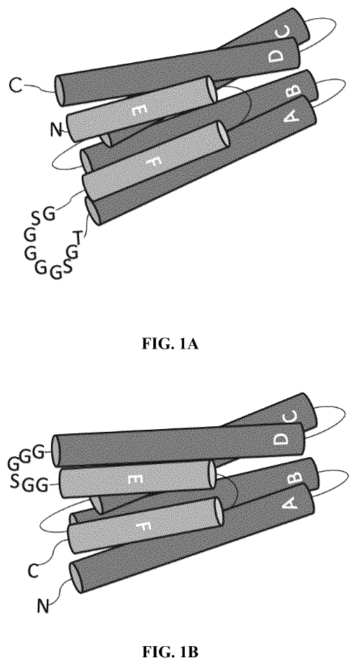

An engineered IL10 polypeptide can comprise, consist essentially of, or consist of, from N-to-C terminus, an E helix, an F helix, an A helix, a B helix, a C helix, and a D helix. An illustrative structure of such an engineered IL10 polypeptide is provided in FIG. 1A, and illustrative sequences are provided in SEQ ID NOs: 3-6. Such an orientation can, for example, reduce formation of IL10 dimers and promote retention of IL10 in a monomeric form. This orientation can, for example, provide a C-terminus that can be accessible for linking to a fusion partner without substantially reducing IL10 activity or with relatively low reduction in IL10 activity (e.g., induction of IL10 receptor signaling). In some embodiments, this arrangement of helices facilitates functionality of the engineered IL10 when the C-terminus of the engineered IL10 is joined to the N-terminus of a fusion partner (for example, joined to one arm of an Fc fragment, optionally via a linker). In some embodiments, this arrangement of helices allows the E and F helices to fold into the A-D helices of the same copy of the engineered polypeptide, while reducing or substantially eliminating formation of dimers with another IL10 monomer.

In some embodiments, the engineered IL10 polypeptide does not contain a second A helix, B helix, C helix, D helix, E helix, and/or F helix. In some embodiments, the engineered IL10 polypeptide does not contain a second A helix. In some embodiments, the engineered IL10 polypeptide does not contain a second B helix. In some embodiments, the engineered IL10 polypeptide does not contain a second C helix. In some embodiments, the engineered IL10 polypeptide does not contain a second D helix. In some embodiments, the engineered IL10 polypeptide does not contain a second E helix. In some embodiments, the engineered IL10 polypeptide does not contain a second F helix.

When any polypeptide or fusion polypeptide disclosed herein contains a linker, the linker may be designed and/or positioned with consideration as to whether or not glycosylation of the resulting polypeptide is desired. For example, use of the linker GSGGGGSG in an engineered IL10 polypeptide can result in an N-glycosylation site at an adjacent Asparagine residue, as in SEQ ID NO: 3. Alternate linkers can be used to avoid introduction of the glycosylation site, e.g., as in SEQ ID NOs: 4-6. In some embodiments, a linker is incorporated such that a glycosylation site is introduced in a polypeptide or fusion polypeptide. In some embodiments, a linker is incorporated such that a glycosylation site is not introduced in a polypeptide or fusion polypeptide.

In some embodiments, an engineered IL10 polypeptide comprises an insertion that promotes the engineered IL10 polypeptide folding as a monomer or reduces dimerization or multimerization of the engineered IL10 polypeptide. Wild type IL10 can fold as a stable dimer, with E′-F′ helices of one monomer integrated in the A-D 4-helix structure of the other monomer. In some embodiments, an engineered IL10 polypeptide comprises an insertion between the D and E helices of IL10 (e.g., an insertion of about 6 amino acids, such as GGGSGG (SEQ ID NO: 124) or another linker sequence disclosed herein) to facilitate the E-F helices folding into the 4-helix structure (e.g., hydrophobic core) created by helices A-D of the same polypeptide, promoting maintenance of the monomeric form of IL10. An illustrative structure of such an engineered IL10 polypeptide is provided in FIG. 1B, and an illustrative sequence in SEQ ID NO: 8.

Illustrative examples of engineered IL10 polypeptide sequences are provided in TABLE 2. Linker sequences are shown b underline.

TABLE 2

| SEQ | | |
|---|---|---|
| ID NO: | Description | Sequence |
| 3 | IL10 | KSKAVEQVKNAFNKLQEKGIYKAMSEFDIFINYIEAYMTMKIR |
| | monomer | NGSGGGGSGTSPGQGTQSENSCTHFPGNLPNMLRDLRDAFSRV |
| | | KTFFQMKDQLDNLLLKESLLEDFKGYLGCQALSEMIQFYLEEV |
| | | MPQAENQDPDIKAHVNSLGENLKTLRLRLRRCHRFLPCEN |
| 4 | IL10 | KSKAVEQVKNAFNKLQEKGIYKAMSEFDIFINYIEAYMTMKIR |
| | monomer | NTGSGGGGSGSPGQGTQSENSCTHFPGNLPNMLRDLRDAFSRV |
| | | KTFFQMKDQLDNLLLKESLLEDFKGYLGCQALSEMIQFYLEEV |
| | | MPQAENQDPDIKAHVNSLGENLKTLRLRLRRCHRFLPCEN |
| 5 | IL10 | KSKAVEQVKNAFNKLQEKGIYKAMSEFDIFINYIEAYMTMKIR |
| | monomer | NGGSGGGSGTSPGQGTQSENSCTHFPGNLPNMLRDLRDAFSRV |
| | | KTFFQMKDQLDNLLLKESLLEDFKGYLGCQALSEMIQFYLEEV |
| | | MPQAENQDPDIKAHVNSLGENLKTLRLRLRRCHRFLPCEN |
| 6 | IL10 | KSKAVEQVKNAFNKLQEKGIYKAMSEFDIFINYIEAYMTMKIR |
| | monomer | NGGSGGGSGSPGQGTQSENSCTHFPGNLPNMLRDLRDAFSRV |
| | | KTFFQMKDQLDNLLLKESLLEDFKGYLGCQALSEMIQFYLEEV |
| | | MPQAENQDPDIKAHVNSLGENLKTLRLRLRRCHRFLPCEN |
| 7 | IL10 | KSKAVEQVKNAFNKLQEKGIYKAMSEFDIFINYIEAYMTMKIR |
| | monomer | NGSGGGGSGTSPGQGTQSENSCTHFPGNLPNMLRDLRDAFSRV |
| | with C- | KTFFQMKDQLDNLLLKESLLEDFKGYLGCQALSEMIQFYLEEV |
| | terminal | MPQAENQDPDIKAHVNSLGENLKTLRLRLRRCHRFLPCENHH |
| | 6xHis tag | HHHH |
| 8 | IL10 | SPGQGTQSENSCTHFPGNLPNMLRDLRDAFSRVKTFFQMKDQ |
| | Josephson | LDNLLLKESLLEDFKGYLGCQALSEMIQFYLEEVMPQAENQDP |
| | monomer | DIKAHVNSLGENLKTLRLRLRRCHRFLPCENGGGSGGKSKAVE |
| | | QVKNAFNKLQEKGIYKAMSEFDIFINYIEAYMTMKIRN |
| 9 | IL10 | HHHHHHSPGQGTQSENSCTHFPGNLPNMLRDLRDAFSRVKTFF |
| | Josephson | QMKDQLDNLLLKESLLEDFKGYLGCQALSEMIQFYLEEVMPQ |
| | monomer | AENQDPDIKAHVNSLGENLKTLRLRLRRCHRFLPCENGGGSGG |
| | with N- | KSKAVEQVKNAFNKLQEKGIYKAMSEFDIFINYIEAYMTMKIR |
| | terminal | N |
| | 6xHis tag | |

An IL10 polypeptide or engineered IL10 polypeptide disclosed herein can comprise, consist essentially of, or consist of an amino acid sequence with at least about 7000, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 1-9.

An IL10 polypeptide or engineered IL10 polypeptide disclosed herein can comprise, consist essentially of, or consist of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 1-9.

In some embodiments, an IL10 polypeptide or engineered IL10 polypeptide comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity or sequence similarity to any one of SEQ ID NOs: 1-9.

In some embodiments, the IL10 polypeptide or engineered IL10 polypeptide comprises, consists essentially of, or consists of the amino acid sequence of any one of SEQ ID NOs: 1-9.

In some embodiments, the IL10 polypeptide or engineered IL10 polypeptide comprises an amino acid sequence with one or more insertions, deletions, and/or substitutions relative to any one of SEQ ID NOs: 1-9.

For example, the IL10 polypeptide or engineered IL10 polypeptide can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 1-9.

In some embodiments, the IL10 polypeptide or engineered IL10 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 1-9.

In some embodiments, the IL10 polypeptide or engineered IL10 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 1-9.

The one or more insertions can be at the N-terminus, the C-terminus, within the amino acid sequence, or a combination thereof. The one or more insertions can be contiguous, non-contiguous, or a combination thereof.

In some embodiments, the IL10 polypeptide or engineered IL10 polypeptide comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 1-9.

In some embodiments, the IL10 polypeptide or engineered IL10 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 1-9.

In some embodiments, the IL10 polypeptide or engineered IL10 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 1-9.

The one or more deletions can be at the N-terminus, the C-terminus, within the amino acid sequence, or a combination thereof. The one or more deletions can be contiguous, non-contiguous, or a combination thereof.

In some embodiments, the IL10 polypeptide or engineered IL10 polypeptide comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 1-9.

In some embodiments, the IL10 polypeptide or engineered IL10 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 1-9.

In some embodiments, the IL10 polypeptide or engineered IL10 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 1-9.

The one or more substitutions can be at the N-terminus, the C-terminus, within the amino acid sequence, or a combination thereof. The one or more substitutions can be contiguous, non-contiguous, or a combination thereof.

### B. Interleukin 4 (IL4)

Compositions and methods disclosed herein can comprise an interleukin 4 (IL4) polypeptide. An IL4 polypeptide can be a mammalian or a human IL4 polypeptide, a variant thereof, or a functional fragment thereof. In some embodiments, an IL4 polypeptide is a human IL4 polypeptide, or a variant or functional fragment thereof. In some embodiments, an IL4 polypeptide is a murine, rodent, canine, feline, equine, porcine, primate, or bovine IL4 polypeptide, or a variant or functional fragment thereof. In some embodiments, an IL4 polypeptide is a non-human and/or non-rodent mammalian IL4 polypeptide, or a variant or functional fragment thereof.

Non-limiting examples of amino acid sequences representing human IL4 are set forth in SEQ ID NOs: 10-13.

An IL4 polypeptide of the disclosure can comprise a wild type IL4 sequence. Non-limiting examples of wild type IL4 sequences include SEQ ID NOs: 10-13. A canonical wild type IL4 sequence of the disclosure can be SEQ ID NO: 10.

An IL4 polypeptide of the disclosure can comprise an IL4 variant, derivative, or fragment thereof with one or more amino acid substitutions. For example, an IL4 variant, derivative, or fragment thereof can comprise an amino acid substitution at position K117, T118, R121, E122, Y124, S125, S128, S129, or a combination thereof relative to SEQ ID NO: 10. In some embodiments, an IL4 variant, derivative, or fragment thereof comprises a substitution that is K117R, T118V, R121Q, R121D, R121K, R121E, E122S, Y124W, Y124F, Y124D, S125F, S128G, S125R, S129A, or a combination thereof relative to SEQ ID NO: 10. In some embodiments, an IL4 variant, derivative, or fragment thereof comprises the substitutions K117R, T118V, R121Q, E122S, Y124W, S125F, S128G, and S129A relative to SEQ ID NO: 10. In some embodiments, an IL4 variant, derivative, or fragment thereof comprises the substitutions R121D and Y124D relative to SEQ ID NO: 10.

In some embodiments, an IL4 variant, derivative, or fragment thereof does not contain a substitution at position K117, T118, R121, E122, Y124, 5125, 5128, or 5129, relative to SEQ ID NO: 10. In some embodiments, an IL4 variant, derivative, or fragment thereof does not contain a K117R, T118V, R121Q, R121D, R121K, R121E, E122S, Y124W, Y124F, Y124D, S125F, S128G, S125R, or S129A substitution.

An IL4 polypeptide, variant, derivative, or fragment thereof can bind to an IL4 receptor. An IL4 receptor can comprise, for example, interleukin 4 receptor alpha (IL-4Rα), interleukin 13 receptor alpha 1 (IL-13Rα1), common gamma chain, or a combination thereof. An IL4 receptor can comprise IL-4Rα and IL-13Rα1 subunits. An IL4 receptor can comprise IL-4Rα and common gamma chain subunits.

In some embodiments, an IL4 polypeptide or IL4 variant, derivative, or fragment thereof of the disclosure can activate a native IL4 receptor. A native IL4 receptor can be, for example, a receptor comprising IL-4Rα and IL-13Rα1 subunits, or IL-4Rα and common gamma chain subunits. In some embodiments, an IL4 or IL4 variant, derivative, or fragment thereof of the disclosure can activate a native IL4 receptor when present in a fusion polypeptide disclosed herein, for example, fused to IL10 via a linker, fused to IL13 via a linker, or fused to an immunoglobulin constant domain (e.g., fused to an Fc domain on one chain of a heterodimeric immunocytokine).

An IL4 polypeptide, variant, derivative, or fragment thereof can bind to one or more IL4 receptor subunits, for example, IL-4Rα, IL-13Rα1, common gamma chain, or a combination thereof. In some embodiments an IL4 polypeptide binds to IL-4Rα. In some embodiments an IL4 polypeptide binds to IL-13Rα1. In some embodiments an IL4 polypeptide binds to common gamma chain. In some embodiments an IL4 polypeptide binds to IL-4Rα, IL-13Rα1, and common gamma chain. In some embodiments an IL4 polypeptide binds to IL-4Rα and IL-13Rα1. In some embodiments an IL4 polypeptide binds to IL-4Rα and common gamma chain.

In some embodiments, an IL4 polypeptide or IL4 variant, derivative, or fragment thereof of the disclosure binds to an IL4 receptor subunit with about a comparable affinity as a wild type IL4 polypeptide. A comparable affinity can be, for example, less than about 50, less than about 25, less than about 10, less than about 5, less than about 2, less than about 1.5, or less than about 1.1 fold increased affinity compared to a wild type IL4 sequence. A comparable affinity can be, for example, less than about 50, less than about 25, less than about 10, less than about 5, less than about 2, less than about 1.5, or less than about 1.1 fold decreased affinity compared to a wild type IL4 sequence. In some embodiments, an IL4 or IL4 variant, derivative, or fragment thereof of the disclosure can bind to IL-4Rα, IL-13Rα1, and/or common gamma chain with at least a comparable affinity as a wild type IL4. In some embodiments, an IL4 or IL4 variant, derivative, or fragment thereof of the disclosure can bind to IL-4Rα, IL-13Rα1, and/or common gamma chain with at most a comparable affinity as a wild type IL4.

In some embodiments, an IL4 or IL4 variant, derivative, or fragment thereof can bind to an IL4 receptor or IL4 receptor subunit with at least 1.5 fold, at least 2 fold, at least 5 fold, at least 10 fold, at least 20 fold, at least 50 fold, at least 100 fold, at least 500 fold, at least 1000 fold, or at least 10,000 fold increased affinity relative to a wild type IL4 sequence. In some embodiments, an IL4 or IL4 variant, derivative, or fragment thereof can bind to an IL4 receptor or IL4 receptor subunit with at least 1.5 fold, at least 2 fold, at least 5 fold, at least 10 fold, at least 20 fold, at least 50 fold, at least 100 fold, at least 500 fold, at least 1000 fold, or at least 10,000 fold decreased affinity relative to a wild type IL4 sequence.

In some embodiments, a polypeptide of the disclosure does not contain IL4.

Illustrative IL4 amino acid sequences are provided in TABLE 3.

TABLE 3

| SEQ | | |
|---|---|---|
| ID NO: | Description | Sequence |
| 10 | IL4 | HKCDITLQEIIKTLNSLTEQKTLCT |
| | | ELTVTDIFAASKNTTEKETFCRAAT |
| | | VLRQFYSHHEKDTRCLGATAQQFHR |
| | | HKQLIRFLKRLDRNLWGLAGLNSCP |
| | | VKEANQSTLENFLERLKTIMREKYS |
| | | KCSS |
| 11 | IL4 | HKCDITLQEIIKTLNSLTEQKNTTE |
| | (delta 2 | KETFCRAATVLRQFYSHHEKDTRCL |
| | splice | GATAQQFHRHKQLIRFLKRLDRNLW |
| | variant) | GLAGLNSCPVKEANQSTLENFLERL |
| | | KTIMREKYSKCSS |
| 12 | IL4 | HKRDITLQEIIKTLNSLTEQKTLCT |
| | | ELTVTDIFAASKNTTEKETFCRAAT |
| | | VLRQFYSHHEKDTRCLGATAQQFHR |
| | | HKQLIRFLKRLDRNLWGLAGLNSCP |
| | | VKEANQSTLENFLERLKTIMREKYS |
| | | KCSS |
| 13 | IL4 | HKRDITLQEIIKTLNSLTEQKNTTE |
| | | KETFCRAATVLRQFYSHHEKDTRCL |
| | | GATAQQFHRHKQLIRFLKRLDRNLW |
| | | GLAGLNSCPVKEANQSTLENFLERL |
| | | KTIMREKYSKCSS |

An IL4 polypeptide disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 10-13.

An IL4 polypeptide disclosed herein can comprise, consist essentially of, or consist of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 10-13.

In some embodiments, an IL4 polypeptide comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity or sequence similarity to any one of SEQ ID NOs: 10-13.

In some embodiments, the IL4 polypeptide comprises, consists essentially of, or consists of the amino acid sequence of any one of SEQ ID NOs: 10-13.

In some embodiments, the IL4 polypeptide comprises an amino acid sequence with one or more insertions, deletions, and/or substitutions relative to any one of SEQ ID NOs: 10-13.

For example, the IL4 polypeptide can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 10-13.

In some embodiments, the IL4 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 10-13.

In some embodiments, the IL4 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 10-13.

The one or more insertions can be at the N-terminus, the C-terminus, within the amino acid sequence, or a combination thereof. The one or more insertions can be contiguous, non-contiguous, or a combination thereof.

In some embodiments, the IL4 polypeptide comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 10-13.

In some embodiments, the IL4 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 10-13.

In some embodiments, the IL4 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 10-13.

The one or more deletions can be at the N-terminus, the C-terminus, within the amino acid sequence, or a combination thereof. The one or more deletions can be contiguous, non-contiguous, or a combination thereof.

In some embodiments, the IL4 polypeptide comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 10-13.

In some embodiments, the IL4 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 10-13.

In some embodiments, the IL4 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 10-13.

The one or more substitutions can be at the N-terminus, the C-terminus, within the amino acid sequence, or a combination thereof. The one or more substitutions can be contiguous, non-contiguous, or a combination thereof.

### C. Interleukin 13 (IL13)

Compositions and methods disclosed herein can comprise an interleukin 13 (IL13) polypeptide. An IL13 polypeptide can be a mammalian or a human IL13 polypeptide, a variant thereof (for example, an engineered IL13 polypeptide), or a functional fragment thereof. In some embodiments, an IL13 polypeptide is a human IL13 polypeptide, or a variant or functional fragment thereof. In some embodiments, an IL13 polypeptide is a murine, rodent, canine, feline, equine, porcine, primate, or bovine IL13 polypeptide, or a variant or functional fragment thereof. In some embodiments, an IL13 polypeptide is a non-human and/or non-rodent mammalian IL13 polypeptide, or a variant or functional fragment thereof.

Non-limiting examples of amino acid sequences representing human IL13 are set forth in SEQ ID NOs: 14-21.

An IL13 of the disclosure can comprise a wild type IL13 sequence. Non-limiting examples of wild type IL13 sequences include SEQ ID NOs: 14-21. SEQ ID NO: 19 can be a canonical wild type IL13 sequence of the disclosure.

An IL13 polypeptide of the disclosure can comprise an IL13 variant, derivative, or fragment thereof with one or more amino acid substitutions. For example, an IL13 variant, derivative, or fragment thereof can comprise an amino acid substitution at position L10, E12, R11, I14, E15, E16, V18, R65, S68, R86, D87, T88, K89, D98, L101, L103, K104, K105 L106, F107, R108, R111, F114, N113, or a combination thereof relative to SEQ ID NO: 15 or SEQ ID NO: 19. In some embodiments, an IL13 variant, derivative, or fragment thereof comprises a substitution that is L10F, L10I, L10V, L10A, L10D, L10T, L10H, R11S, R11N, R11H, R11L, R11I, I14L, I14F, I14V, I14M, V18L, V18F, V18I, E12A, R65D, R86K, R86T, R86M, D87E, D87K, D87R, D87G, D87S, T88S, T88I, T88K, T88R, K89R, K89T, K89M, L101F, L101I, L101Y, L101H, L101N, K104R, K104T, K104M, K105T, K105A, K105R, K105E, F107L, F107I, F107V, F107M, R108K, R108T, R108M, E12K, E12I, E12C, E12S, E12R, E12Y, E12D, E15K, E16K, R65D, S68D, D98K, L101A, L103A, K104D, K105D, L106A, F107Y, R108D, R111D, F114D, N113D, or a combination thereof relative to SEQ ID NO: 15 or SEQ ID NO: 19. In some embodiments, an IL13 variant, derivative, or fragment thereof comprises the substitutions L10H, R86T, D87G, T88R, and R108K relative to SEQ ID NO: 15 or SEQ ID NO: 19. In some embodiments, an IL13 variant, derivative, or fragment thereof comprises the substitutions L10A, V18F, R86K, D87K, K89R, L101I, K104R, and R108K relative to SEQ ID NO: 15 or SEQ ID NO: 19. In some embodiments, an IL13 variant, derivative, or fragment thereof comprises the substitutions R11S, V18I, R86K, D87G, T88S, K89M, L101Y, K104R, and K105T relative to SEQ ID NO: 15 or SEQ ID NO: 19. In some embodiments, an IL13 variant, derivative, or fragment thereof comprises the substitutions L10V, K89R, L101N, K105E, and R108T relative to SEQ ID NO: 15 or SEQ ID NO: 19. In some embodiments, an IL13 variant, derivative, or fragment thereof comprises the substitutions L10D, R11I, V18I, R86K, D87K, K89R, and R108K relative to SEQ ID NO: 15 or SEQ ID NO: 19. In some embodiments, an IL13 variant, derivative, or fragment thereof comprises the substitutions L10A, R86T, D87G, T88K, K89R, L101N, K104R, K105A, and R108K relative to SEQ ID NO: 15 or SEQ ID NO: 19. In some embodiments, an IL13 variant, derivative, or fragment thereof comprises the substitutions L10V, K89R, L101N, K105E, and R108T relative to SEQ ID NO: 15 or SEQ ID NO: 19. In some embodiments, an IL13 variant, derivative, or fragment thereof comprises the substitutions R11S, I14M, T88S, L101N, K105A, and R108K relative to SEQ ID NO: 15 or SEQ ID NO: 19. In some embodiments, an IL13 variant, derivative, or fragment thereof comprises the substitutions L10H, R11L, V18I, R86K, D87E, K89R, L101N, K105T, and R108K relative to SEQ ID NO: 15 or SEQ ID NO: 19. In some embodiments, an IL13 variant, derivative, or fragment thereof comprises the substitutions L10H, R86T, D87G, T88R, and R108K relative to SEQ ID NO: 15 or SEQ ID NO: 19. In some embodiments, an IL13 variant, derivative, or fragment thereof comprises the substitutions L10A, V18F, R86K, D87K, K89R, L101I, K104R, and R108K relative to SEQ ID NO: 15 or SEQ ID NO: 19. In some embodiments, an IL13 variant, derivative, or fragment thereof comprises the substitutions L10T or L10D, R11I, V18I, R86K, D87K or D87G, T88S, K89R, L101Y, K104R, K105T, and R108K relative to SEQ ID NO: 15 or SEQ ID NO: 19. In some embodiments, an IL13 variant, derivative, or fragment thereof comprises the substitutions L10A or L10V, R86T, D87G, T88K, K89R, L101N, K104R, K105A or K105E, and R108K or R108T relative to SEQ ID NO: 15 or SEQ ID NO: 19. In some embodiments, an IL13 variant, derivative, or fragment thereof comprises the substitutions L10V, V18I, D87S, D88S, L101F, K104R, and K105T relative to SEQ ID NO: 15 or SEQ ID NO: 19. In some embodiments, an IL13 variant, derivative, or fragment thereof comprises the substitutions R11S, V18I, R86K, D87G, T88S, K89M, L101Y, K104R, and K105T relative to SEQ ID NO: 15 or SEQ ID NO: 19. In some embodiments, an IL13 variant, derivative, or fragment thereof comprises the substitutions L10V, V18I, D87S, T88S, L101F, K104R, and K105T relative to SEQ ID NO: 15 or SEQ ID NO: 19. In some embodiments, an IL13 variant, derivative, or fragment thereof comprises the substitutions L10V or L10I, D87S, T88S, K89R, L101H or L101F, K104R, and K105T relative to SEQ ID NO: 15 or SEQ ID NO: 19. In some embodiments, an IL13 variant, derivative, or fragment thereof comprises the substitutions L10I, V18I, R86T, D87G, T88S, K89R, L101Y, L101H, K104R, and K105A relative to SEQ ID NO: 15 or SEQ ID NO: 19. In some embodiments, an IL13 variant, derivative, or fragment thereof comprises the substitutions L10V, V18I, D87S, T88S, L101F, K104R, and K105T relative to SEQ ID NO: 15 or SEQ ID NO: 19. In some embodiments, an IL13 variant, derivative, or fragment thereof comprises the substitutions V18I, R86T, D87G, T88S, L101Y, K104R, and K105A relative to SEQ ID NO: 15 or SEQ ID NO: 19. In some embodiments, an IL13 variant, derivative, or fragment thereof comprises the substitutions R11I, V18I, R86K, D87G, T88S, L101H, K104R, K105A, and F107M relative to SEQ ID NO: 15 or SEQ ID NO: 19. In some embodiments, an IL13 variant, derivative, or fragment thereof comprises the substitutions E12K and S68D relative to SEQ ID NO: 15 or SEQ ID NO: 19. In some embodiments, an IL13 variant, derivative, or fragment thereof comprises the substitutions E12K and R108D relative to SEQ ID NO: 15 or SEQ ID NO: 19. In some embodiments, an IL13 variant, derivative, or fragment thereof comprises the substitutions E12K and R111D relative to SEQ ID NO: 15 or SEQ ID NO: 19. In some embodiments, an IL13 variant, derivative, or fragment thereof comprises the substitutions E12Y and R65D relative to SEQ ID NO: 15 or SEQ ID NO: 19. In some embodiments, an IL13 variant, derivative, or fragment thereof comprises the substitutions E12Y and S68D relative to SEQ ID NO: 15 or SEQ ID NO: 19. In some embodiments, an IL13 variant, derivative, or fragment thereof comprises the substitutions E12K, R65D and S68D relative to SEQ ID NO: 15 or SEQ ID NO: 19. In some embodiments, an IL13 variant, derivative, or fragment thereof comprises the substitutions E12Y, R65D and S68D relative to SEQ ID NO: 15 or SEQ ID NO: 19. In some embodiments, an IL13 variant, derivative, or fragment thereof comprises the substitutions E12K, R65D, S68D and R111D relative to SEQ ID NO: 15 or SEQ ID NO: 19.

In some embodiments, an IL13 variant, derivative, or fragment thereof does not contain a substitution at position L10, E12, R11, I14, E15, E16, V18, R65, S68, R86, D87, T88, K89, D98, L101, L103, K104, K105 L106, F107, R108, R111, F114, or N113 relative to SEQ ID NO: 15 or SEQ ID NO: 19. In some embodiments, an IL13 variant, derivative, or fragment thereof does not contain a L10F, L10I, L10V, L10A, L10D, L10T, L10H, R11S, R11N, R11H, R11L, R11, I14L, I14F, I14V, I14M, V18L, V18F, V18I, E12A, R65D, R86K, R86T, R86M, D87E, D87K, D87R, D87G, D87S, T88S, T88I, T88K, T88R, K89R, K89T, K89M, L101F, L101I, L101Y, L101H, L101N, K104R, K104T, K104M, K105T, K105A, K105R, K105E, F107L, F107I, F107V, F107M, R108K, R108T, R108M, E12K, E12I, E12C, E12S, E12R, E12Y, E12D, E15K, E16K, R65D, S68D, D98K, L101A, L103A, K104D, K105D, L106A, F107Y, R108D, R111D, F114D, or N113D substitution.

Where a substitution is described herein, it can be adjusted depending on the sequence it is applied to compared to the reference or canonical sequence. For example, The N-terminus of SEQ ID NOs: 14-16 varies, such that an L10F substitution relative to SEQ ID NO: 15 is an L to F substitution in L11 of SEQ ID NO: 14 and L9 in SEQ ID NO: 16.

An IL13 polypeptide, variant, derivative, or fragment thereof can bind to an IL13 receptor. An IL13 receptor can comprise, for example, interleukin 13 receptor alpha 1 (IL-13Rα1), interleukin 13 receptor alpha 2 (IL-13Rα2), interleukin 4 receptor alpha (IL-4Rα), or a combination thereof. In some embodiments, an IL13 or IL13 variant, derivative, or fragment thereof of the disclosure can activate a native IL13 receptor. A native IL13 receptor can be, for example, a receptor comprising an IL-13Rα1 subunit and an IL-4Rα subunit. In some embodiments, an IL13 or IL13 variant, derivative, or fragment thereof of the disclosure can activate a native IL13 receptor when present in a fusion polypeptide disclosed herein, for example, fused to IL4 via a linker, fused to IL10 via a linker, or fused to an immunoglobulin constant domain (e.g., fused to an Fc domain on one chain of a heterodimeric immunocytokine).

An IL13 polypeptide, variant, derivative, or fragment thereof can bind to one or more IL13 receptor subunits, for example, IL-13Rα1, IL-13Rα2, IL-4Rα, or a combination thereof. In some embodiments an IL13 polypeptide binds to IL-13Rα1. In some embodiments an IL13 polypeptide binds to IL-13Rα2. In some embodiments an IL13 polypeptide binds to IL-4Rα. In some embodiments an IL13 polypeptide binds to IL-13Rα1 and IL-4Rα. In some embodiments an IL13 polypeptide binds to IL-13Rα1 and IL-13Rα2. In some embodiments an IL13 polypeptide binds to IL-13Rα2 and IL-4Rα. In some embodiments an IL13 polypeptide binds to IL-13Rα1, IL-13Rα2, and IL-4Rα.

In some embodiments, an IL13 polypeptide or IL13 variant, derivative, or fragment thereof of the disclosure binds to an IL13 receptor subunit with about a comparable affinity as a wild type IL13 sequence. A comparable affinity can be, for example, less than about 50, less than about 25, less than about 10, less than about 5, less than about 2, less than about 1.5, or less than about 1.1 fold increased affinity compared to a wild type IL13 sequence. A comparable affinity can be, for example, less than about 50, less than about 25, less than about 10, less than about 5, less than about 2, less than about 1.5, or less than about 1.1 fold decreased affinity compared to a wild type IL13 sequence. In some embodiments, an IL13 or IL13 variant, derivative, or fragment thereof of the disclosure can bind to an IL13 receptor subunit with at least a comparable affinity as a wild type IL13. In some embodiments, an IL13 or IL13 variant, derivative, or fragment thereof of the disclosure can bind to an IL13 receptor subunit with at most a comparable affinity as a wild type IL13.

In some embodiments, an IL13 or IL13 variant, derivative, or fragment thereof can bind to an IL13 receptor or IL13 receptor subunit with at least 1.5 fold, at least 2 fold, at least 5 fold, at least 10 fold, at least 20 fold, at least 50 fold, at least 100 fold, at least 500 fold, at least 1000 fold, or at least 10,000 fold increased affinity relative to a wild type IL13 sequence. In some embodiments, an IL13 or IL13 variant, derivative, or fragment thereof can bind to an IL13 receptor or IL13 receptor subunit with at least 1.5 fold, at least 2 fold, at least 5 fold, at least 10 fold, at least 20 fold, at least 50 fold, at least 100 fold, at least 500 fold, at least 1000 fold, or at least 10,000 fold decreased affinity relative to a wild type IL13 sequence. For example, in some embodiments the IL13, variant, derivative, or fragment thereof can bind to IL-13Rα2 with a reduced affinity relative to wild type IL13.

In some embodiments, a polypeptide of the disclosure does not contain IL13.

Illustrative IL13 sequences are provided in TABLE 4.

TABLE 4

| SEQ | | |
|---|---|---|
| ID NO: | Description | Sequence |
| 14 | IL13 | SPGPVPPSTALRELIEELVNITQNQ |
| | | KAPLCNGSMVWSINLTAGMYCAALE |
| | | SLINVSGCSAIEKTQRMLSGFCPHK |
| | | VSAGQFSSLHVRDTKIEVAQFVKDL |
| | | LLHLKKLFREGRFN |
| 15 | IL13 | PGPVPPSTALRELIEELVNITQNQK |
| | | APLCNGSMVWSINLTAGMYCAALES |
| | | LINVSGCSAIEKTQRMLSGFCPHKV |
| | | SAGQFSSLHVRDTKIEVAQFVKDLL |
| | | LHLKKLFREGQFN |
| 16 | IL13 | GPVPPSTALRELIEELVNITQNQKA |
| | | PLCNGSMVWSINLTAGMYCAALESL |
| | | INVSGCSAIEKTQRMLSGFCPHKVS |
| | | AGQFSSLHVRDTKIEVAQFVKDLLL |
| | | HLKKLFREGQFN |
| 17 | IL13 | SPGPVPPSTALRELIEELVNITQNQ |
| | | KAPLCNGSMVWSINLTAGMYCAALE |
| | | SLINVSGCSAIEKTQRMLSGFCPHK |
| | | VSAGQFSSLHVRDTKIEVAQFVKDL |
| | | LLHLKKLFREGQFN |
| 18 | IL 13 | LTCLGGFASPGPVPPSTALRELIEE |
| | | LVNITQNQKAPLCNGSMVWSINLTA |
| | | GMYCAALESLINVSGCSAIEKTQRM |
| | | LSGFCPHKVSAGQFSSLHVRDTKIE |
| | | VAQFVKDLLLHLKKLFREGQFN |
| 19 | IL13 | PGPVPPSTALRELIEELVNITQNQK |
| | | APLCNGSMVWSINLTAGMYCAALES |
| | | LINVSGCSAIEKTQRMLSGFCPHKV |
| | | SAGQFSSLHVRDTKIEVAQFVKDLL |
| | | LHLKKLFREGRFN |
| 20 | IL13 | GPVPPSTALRELIEELVNITQNQKA |
| | | PLCNGSMVWSINLTAGMYCAALESL |
| | | INVSGCSAIEKTQRMLSGFCPHKVS |
| | | AGQFSSLHVRDTKIEVAQFVKDLLL |
| | | HLKKLFREGRFN |
| 21 | IL 13 | LTCLGGFASPGPVPPSTALRELIEE |
| | | LVNITQNQKAPLCNGSMVWSINLTA |
| | | GMYCAALESLINVSGCSAIEKTQRM |
| | | LSGFCPHKVSAGQFSSLHVRDTKIE |
| | | VAQFVKDLLLHLKKLFREGRFN |

An IL13 polypeptide disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 14-21.

An IL13 polypeptide disclosed herein can comprise, consist essentially of, or consist of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 14-21.

In some embodiments, an IL13 polypeptide comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity or sequence similarity to any one of SEQ ID NOs: 14-21.

In some embodiments, the IL13 polypeptide comprises, consists essentially of, or consists of the amino acid sequence of any one of SEQ ID NOs: 14-21.

In some embodiments, the IL13 polypeptide comprises an amino acid sequence with one or more insertions, deletions, and/or substitutions relative to any one of SEQ ID NOs: 14-21.

For example, the IL13 polypeptide can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 14-21.

In some embodiments, the IL13 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 14-21.

In some embodiments, the IL13 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 14-21.

The one or more insertions can be at the N-terminus, the C-terminus, within the amino acid sequence, or a combination thereof. The one or more insertions can be contiguous, non-contiguous, or a combination thereof.

In some embodiments, the IL13 polypeptide comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 14-21.

In some embodiments, the IL13 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 14-21.

In some embodiments, the IL13 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 14-21.

The one or more deletions can be at the N-terminus, the C-terminus, within the amino acid sequence, or a combination thereof. The one or more deletions can be contiguous, non-contiguous, or a combination thereof.

In some embodiments, the IL13 polypeptide comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 14-21.

In some embodiments, the IL13 polypeptide comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 14-21.

In some embodiments, the IL13 polypeptide comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 14-21.

The one or more substitutions can be at the N-terminus, the C-terminus, within the amino acid sequence, or a combination thereof. The one or more substitutions can be contiguous, non-contiguous, or a combination thereof.

### D. Additional Regulatory Cytokines

Compositions and methods disclosed herein can comprise a cytokine, such as a regulatory cytokine or anti-inflammatory cytokine. A cytokine used in a composition (e.g., fusion polypeptide or engineered immunocytokine) disclosed herein can be a mammalian or human cytokine, a variant thereof, or a functional fragment thereof. In some embodiments, a cytokine used in a composition (e.g., fusion polypeptide or engineered immunocytokine) disclosed herein is a human cytokine, or a variant or functional fragment thereof. In some embodiments, a cytokine used in a composition (e.g., fusion polypeptide or engineered immunocytokine) disclosed herein is a murine, rodent, canine, feline, equine, porcine, primate, or bovine cytokine, or a variant or functional fragment thereof. In some embodiments, the cytokine is a non-human and/or non-rodent cytokine, or a variant or functional fragment thereof. The cytokine can be a wild type cytokine. In some embodiments the cytokine is not a wild type cytokine. Illustrative, non-limiting examples of cytokines that can be used include IL27, IL27A, IL27B, IL33, TGFβ1, TGFβ2.

In some embodiments, a composition (e.g., fusion polypeptide or engineered immunocytokine) comprises IL27. In some embodiments, an IL27 of the disclosure comprises an IL27A subunit. In some embodiments, an IL27 of the disclosure comprises a variant IL27A subunit as disclosed below. In some embodiments, an IL27 of the disclosure comprises an IL27B subunit. The IL27 can be, for example, a first cytokine or a second cytokine in an engineered heterodimeric immunocytokine disclosed herein.

In some embodiments, a composition (e.g., fusion polypeptide or engineered immunocytokine) comprises IL27A or a variant or functional fragment thereof. The IL2A7 can be, for example, a first cytokine or a second cytokine in an engineered heterodimeric immunocytokine disclosed herein. Non-limiting examples of IL27A amino acid sequence include SEQ ID NOs: 125 & 126. In some embodiments, the IL27A is a wild type IL27A. An example of an IL27 variant, derivative, or fragment thereof of the disclosure is an IL27 variant sequence that can be secreted as a functional immune modulatory monomer protein, for example, an IL27A variant, derivative, or fragment thereof that can be secreted and function as a functional immune modulatory monomer protein without needing to associate with an IL27B (EBI3) subunit. One or more amino acid substitutions, deletions, or insertions can be introduced to generate such a molecule. SEQ ID NO: 126 is an example of an IL27 variant, derivative, or fragment thereof of the disclosure that comprises one amino acid substation L134C relative to SEQ ID NO: 125 (which is L162C in the sequence that includes the signal peptide), and can be secreted as a functional immune modulatory monomer protein. In some embodiments, the IL27A is a IL27A variant, for example, comprising an amino acid substitution at position F132, N132, L134, P135, E136, E137, L152, L153, P154, or a combination thereof of relative to SEQ ID NO: 125. In some embodiments, an IL27 variant, derivative, or fragment thereof comprises a substitution that is F132C, N132C, L134C, P135C, E136C, E137C, L152C, L153C, P154C, F132D, N132D, L134D, P135D, E136D, E137D, L152D, L153D, P154D, F132E, N132E, L134E, P135E, E136E, E137E, L152E, L153E, P154E, F132R, N132R, L134R, P135R, E136R, E137R, L152R, L153R, P154R, F132K, N132K, L134K, P135K, E136K, E137K, L152K, L153K, P154K, S31A, L91P, or a combination thereof relative to SEQ ID NO: 125. In some embodiments, an IL27 or IL27 variant, derivative, or fragment thereof of the disclosure can activate a native IL27 receptor. A native IL27 receptor can be, for example, a receptor comprising an IL-27RA subunit and a gp130 subunit. In some embodiments, an IL27, IL27A, or variant, derivative, or fragment thereof of the disclosure can activate a native IL27 receptor when present in a fusion polypeptide disclosed herein, for example, fused to an immunoglobulin constant domain (e.g., fused to an Fc domain on one chain of a heterodimeric immunocytokine).

In some embodiments, a composition (e.g., fusion polypeptide or engineered immunocytokine) comprises IL27B or a variant or functional fragment thereof. The IL27B can be, for example, a first cytokine or a second cytokine in an engineered heterodimeric immunocytokine disclosed herein. A non-limiting example of an IL27B amino acid sequence is SEQ ID NOs: 127. In some embodiments, the IL27B is a wild type IL27B. In some embodiments, the IL27B is a IL27B variant, for example, comprising an amino acid substitution relative to SEQ ID NO: 127. In some embodiments, a IL27B variant, derivative, or fragment thereof of the disclosure can activate a native IL27 receptor. A native IL27 receptor can be, for example, a receptor comprising an IL-27RA subunit and a gp130 subunit.

In some embodiments, a composition (e.g., fusion polypeptide or engineered immunocytokine) comprises IL33 or a variant or functional fragment thereof. Non-limiting examples of IL33 amino acid sequence include SEQ ID NOs: 128-134. In some embodiments, the IL33 is a wild type IL33. In some embodiments, the IL33 is an IL33 variant, for example, comprising an amino acid substitution at position 1263 (e.g., an 1263M substitution) relative to SEQ ID NO: 128. In some embodiments, an IL33 or IL33 variant, derivative, or fragment thereof of the disclosure can activate a native IL33 receptor. A native IL33 receptor can be, for example, a receptor comprising an ST2 subunit and an IL1RAP subunit. In some embodiments, an IL33 or variant, derivative, or fragment thereof of the disclosure can activate a native IL33 receptor when present in a fusion polypeptide disclosed herein, for example, fused to an immunoglobulin constant domain (e.g., fused to an Fc domain on one chain of a heterodimeric immunocytokine).

In some embodiments, a composition (e.g., fusion polypeptide or engineered immunocytokine) comprises TGFβ1 or a variant or functional fragment thereof. Non-limiting examples of TGFβ1 amino acid sequences include SEQ ID NOs: 135 & 136. In some embodiments, the TGFβ1 is a wild type TGFβ1. In some embodiments, the TGFβ1 is a TGFβ1 variant. In some embodiments, a TGFβ1 variant, derivative, or fragment thereof of the disclosure can activate a native TGFβ1 receptor. A native TGFβ1 receptor can be, for example, a receptor comprising a TGFβR1 subunit and a TGFβR2 subunit. In some embodiments, a TGFβ1 or TGFβ1 variant, derivative, or fragment thereof of the disclosure can bind to a transforming growth factor beta receptor 1 (TGFβR1), a transforming growth factor beta receptor 2 (TGFβR2), an activin receptor-like kinase 1 (ALK-1), an activin receptor-like kinase 2 (ALK-2), or a combination thereof. In some embodiments, a TGFβ1 or variant, derivative, or fragment thereof of the disclosure can activate a native TGFβ1 receptor when present in a fusion polypeptide disclosed herein, for example, fused to an immunoglobulin constant domain (e.g., fused to an Fc domain on one chain of a heterodimeric immunocytokine).

In some embodiments, a composition (e.g., fusion polypeptide or engineered immunocytokine) comprises TGFβ2 or a variant or functional fragment thereof. Non-limiting examples of TGFβ2 amino acid sequences include SEQ ID NOs: 137-139. In some embodiments, the TGFβ2 is a wild type TGFβ2. In some embodiments, the TGFβ2 is a TGFβ2 variant, for example, comprising an amino acid substitution at position R18 and/or P36 (e.g., an R18C and/or P36H substitution relative to SEQ ID NO: 139. In some embodiments, a TGFβ2 variant, derivative, or fragment thereof of the disclosure can activate a native TGFβ2 receptor. A native TGFβ2 receptor can be, for example, a receptor comprising a TGFβR1 subunit and a TGFβR2 subunit. In some embodiments, a TGFβ2 or TGFβ2 variant, derivative, or fragment thereof can bind to a TGFβR1, TGFβR2, ALK-1, and/or ALK-2. In some embodiments, a TGFβ2 or variant, derivative, or fragment thereof of the disclosure can activate a native TGFβ2 receptor when present in a fusion polypeptide disclosed herein, for example, fused to an immunoglobulin constant domain (e.g., fused to an Fc domain on one chain of a heterodimeric immunocytokine).

In some embodiments, a polypeptide of the disclosure does not contain IL27. In some embodiments, a polypeptide of the disclosure does not contain IL27A. In some embodiments, a polypeptide of the disclosure does not contain IL27B. In some embodiments, a polypeptide of the disclosure does not contain IL33. In some embodiments, a polypeptide of the disclosure does not contain TGFβ1. In some embodiments, a polypeptide of the disclosure does not contain TGFβ2.

Illustrative cytokine amino acid sequences are provided in TABLE 5.

TABLE 5

| SEQ | | |
|---|---|---|
| ID NO: | Description | Sequence |
| 125 | IL27A | FPRPPGRPQLSLQELRREFTVSLHLARKLLSEVRG |
| | | QAHRFAESHLPGVNLYLLPLGEQLPDVSLTFQAWR |
| | | RLSDPERLCFISTTLQPFHALLGGLGTQGRWTNME |
| | | RMQLWAMRLDLRDLQRHLRFQVLAAGFNLPEEEEE |
| | | EEEEEEEERKGLLPGALGSALQGPAQVSWPQLLST |
| | | YRLLHSLELVLSRAVRELLLLSKAGHSVWPLGFPT |
| | | LSPQP |
| 126 | IL27A | FPRPPGRPQLSLQELRREFTVSLHLARKLLSEVRG |
| | (L162C) | QAHRFAESHLPGVNLYLLPLGEQLPDVSLTFQAWR |
| | | RLSDPERLCFISTTLQPFHALLGGLGTQGRWTNME |
| | | RMQLWAMRLDLRDLQRHLRFQVLAAGFNCPEEEEE |
| | | EEEEEEEERKGLLPGALGSALQGPAQVSWPQLLST |
| | | YRLLHSLELVLSRAVRELLLLSKAGHSVWPLGFPT |
| | | LSPQP |
| 127 | IL27B | RKGPPAALTLPRVQCRASRYPIAVDCSWTLPPAPN |
| | | STSPVSFIATYRLGMAARGHSWPCLQQTPTSTSCT |
| | | ITDVQLFSMAPYVLNVTAVHPWGSSSSFVPFITEH |
| | | IIKPDPPEGVRLSPLAERQLQVQWEPPGSWPFPEI |
| | | FSLKYWIRYKRQGAARFHRVGPIEATSFILRAVRP |
| | | RARYYVQVAAQDLTDYGELSDWSLPATATMSLGK |
| 128 | IL33 | MKPKMKYSTNKISTAKWKNTASKALCFKLGKSQQK |
| | | AKEVCPMYFMKLRSGLMIKKEACYFRRETTKRPSL |
| | | KTGRKHKRHLVLAACQQQSTVECFAFGISGVQKYT |
| | | RALHDSSITGISPITEYLASLSTYNDQSITFALED |
| | | ESYEIYVEDLKKDEKKDKVLLSYYESQHPSNESGD |
| | | GVDGKMLMVTLSPTKDFWLHANNKEHSVELHKCEK |
| | | PLPDQAFFVLHNMHSNCVSFECKTDPGVFIGVKDN |
| | | HLALIKVDSSENLCTENILFKLSET |
| 129 | IL33 | AFGISGVQKYTRALHDSSITGISPITEYLASLSTY |
| | | NDQSITFALEDESYEIYVEDLKKDEKKDKVLLSYY |
| | | ESQHPSNESGDGVDGKMLMVTLSPTKDFWLHANNK |
| | | EHSVELHKCEKPLPDQAFFVLHNMHSNCVSFECKT |
| | | DPGVFIGVKDNHLALIKVDSSENLCTENILFKLSE |
| | | T |
| 130 | IL33 | SGVQKYTRALHDSSITGISPITEYLASLSTYNDQS |
| | | ITFALEDESYEIYVEDLKKDEKKDKVLLSYYESQH |
| | | PSNESGDGVDGKMLMVTLSPTKDFWLHANNKEHSV |
| | | ELHKCEKPLPDQAFFVLHNMHSNCVSFECKTDPGV |
| | | FIGVKDNHLALIKVDSSENLCTENILFKLSET |
| 131 | IL33 | HDSSITGISPITEYLASLSTYNDQSITFALEDESY |
| | | EIYVEDLKKDEKKDKVLLSYYESQHPSNESGDGVD |
| | | GKMLMVTLSPTKDFWLHANNKEHSVELHKCEKPLP |
| | | DQAFFVLHNMHSNCVSFECKTDPGVFIGVKDNHLA |
| | | LIKVDSSENLCTENILFKLSET |
| 132 | IL33 | MKPKMKYSTNKISTAKWKNTASKALCFKLGKSQQK |
| | | AKEVCPMYFMKLRSGLMIKKEACYFRRETTKRPSL |
| | | KTGRKHKRHLVLAACQQQSTVECFAFGISGVQKYT |
| | | RALHDSSITDKVLLSYYESQHPSNESGDGVDGKML |
| | | MVTLSPTKDFWLHANNKEHSVELHKCEKPLPDQAF |
| | | FVLHNMHSNCVSFECKTDPGVFIGVKDNHLALIKV |
| | | DSSENLCTENILFKLSET |
| 133 | IL33 | MKPKMKYSTNKISTAKWKNTASKALCFKLGKSQQK |
| | | AKEVCPMYFMKLRSGLMIKKEACYFRRETTKRPSL |
| | | KTGISPITEYLASLSTYNDQSITFALEDESYEIYV |
| | | EDLKKDEKKDKVLLSYYESQHPSNESGDGVDGKML |
| | | MVTLSPTKDFWLHANNKEHSVELHKCEKPLPDQAF |
| | | FVLHNMHSNCVSFECKTDPGVFIGVKDNHLALIKV |
| | | DSSENLCTENILFKLSET |
| 134 | IL33 | MKPKMKYSTNKISTAKWKNTASKALCFKLGNKVLL |
| | | SYYESQHPSNESGDGVDGKMLMVTLSPTKDFWLHA |
| | | NNKEHSVELHKCEKPLPDQAFFVLHNMHSNCVSFE |
| | | CKTDPGVFIGVKDNHLALIKVDSSENLCTENILFK |
| | | LSET |
| 135 | TGFβ1 | MPPSGLRLLPLLLPLLWLLVLTPGRPAAGLSTCKT |
| | (immature) | IDMELVKRKRIEAIRGQILSKLRLASPPSQGEVPP |
| | | GPLPEAVLALYNSTRDRVAGESAEPEPEPEADYYA |
| | | KEVTRVLMVETHNEIYDKFKQSTHSIYMFFNTSEL |
| | | REAVPEPVLLSRAELRLLRLKLKVEQHVELYQKYS |
| | | NNSWRYLSNRLLAPSDSPEWLSFDVTGVVRQWLSR |
| | | GGEIEGFRLSAHCSCDSRDNTLQVDINGFTTGRRG |
| | | DLATIHGMNRPFLLLMATPLERAQHLQSSRHRRAL |
| | | DTNYCFSSTEKNCCVRQLYIDFRKDLGWKWIHEPK |
| | | GYHANFCLGPCPYIWSLDTQYSKVLALYNQHNPGA |
| | | SAAPCCVPQALEPLPIVYYVGRKPKVEQLSNMIVR |
| | | SCKCS |
| 136 | TGFβ1 | ALDTNYCFSSTEKNCCVRQLYIDFRKDLGWKWIHE |
| | (mature) | PKGYHANFCLGPCPYIWSLDTQYSKVLALYNQHNP |
| | | GASAAPCCVPQALEPLPIVYYVGRKPKVEQLSNMI |
| | | VRSCKCS |
| 137 | TGFβ2 | MHYCVLSAFLILHLVTVALSLSTCSTLDMDQFMRK |
| | (immature) | RIEAIRGQILSKLKLTSPPEDYPEPEEVPPEVISI |
| | | YNSTRDLLQEKASRRAAACERERSDEEYYAKEVYK |
| | | IDMPPFFPSETVCPVVTTPSGSVGSLCSRQSQVLC |
| | | GYLDAIPPTFYRPYFRIVRFDVSAMEKNASNLVKA |
| | | EFRVFRLQNPKARVPEQRIELYQILKSKDLTSPTQ |
| | | RYIDSKVVKTRAEGEWLSFDVTDAVHEWLHHKDRN |
| | | LGFKISLHCPCCTFVPSNNYIIPNKSEELEARFAG |
| | | IDGTSTYTSGDQKTIKSTRKKNSGKTPHLLLMLLP |
| | | SYRLESQQTNRRKKRALDAAYCFRNVQDNCCLRPL |
| | | YIDFKRDLGWKWIHEPKGYNANFCAGACPYLWSSD |
| | | TQHSRVLSLYNTINPEASASPCCVSQDLEPLTILY |
| | | YIGKTPKIEQLSNMIVKSCKCS |
| 138 | TGFβ2 | MHYCVLSAFLILHLVTVALSLSTCSTLDMDQFMRK |
| | (immature) | RIEAIRGQILSKLKLTSPPEDYPEPEEVPPEVISI |
| | | YNSTRDLLQEKASRRAAACERERSDEEYYAKEVYK |
| | | IDMPPFFPSENAIPPTFYRPYFRIVRFDVSAMEKN |
| | | ASNLVKAEFRVFRLQNPKARVPEQRIELYQILKSK |
| | | DLTSPTQRYIDSKVVKTRAEGEWLSFDVTDAVHEW |
| | | LHHKDRNLGFKISLHCPCCTFVPSNNYIIPNKSEE |
| | | LEARFAGIDGTSTYTSGDQKTIKSTRKKNSGKTPH |
| | | LLLMLLPSYRLESQQTNRRKKRALDAAYCFRNVQD |
| | | NCCLRPLYIDFKRDLGWKWIHEPKGYNANFCAGAC |
| | | PYLWSSDTQHSRVLSLYNTINPEASASPCCVSQDL |
| | | EPLTILYYIGKTPKIEQLSNMIVKSCKCS |
| 139 | TGFβ2(mature) | ALDAAYCFRNVQDNCCLRPLYIDFKRDLGWKWIHE |
| | | PKGYNANFCAGACPYLWSSDTQHSRVLSLYNTINP |
| | | EASASPCCVSQDLEPLTILYYIGKTPKIEQLSNMI |
| | | VKSCKCS |

A cytokine disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 10000 sequence identity or sequence similarity to any one of SEQ ID NOs: 125-139.

A cytokine disclosed herein can comprise, consist essentially of, or consist of an amino acid sequence with at most about 7000, at most about 710%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 125-139.

In some embodiments, a cytokine comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity or sequence similarity to any one of SEQ ID NOs: 125-139.

In some embodiments, the cytokine comprises, consists essentially of, or consists of the amino acid sequence of any one of SEQ ID NOs: 125-139.

In some embodiments, the cytokine comprises an amino acid sequence with one or more insertions, deletions, and/or substitutions relative to any one of SEQ ID NOs: 125-139.

For example, the cytokine can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 125-139.

In some embodiments, the cytokine comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 125-139.

In some embodiments, the cytokine comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 125-139.

The one or more insertions can be at the N-terminus, the C-terminus, within the amino acid sequence, or a combination thereof. The one or more insertions can be contiguous, non-contiguous, or a combination thereof.

In some embodiments, the cytokine comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 125-139.

In some embodiments, the cytokine comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 125-139.

In some embodiments, the cytokine comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 125-139.

The one or more deletions can be at the N-terminus, the C-terminus, within the amino acid sequence, or a combination thereof. The one or more deletions can be contiguous, non-contiguous, or a combination thereof.

In some embodiments, the cytokine comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 125-139.

In some embodiments, the cytokine comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 125-139.

In some embodiments, the cytokine comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 125-139.

The one or more substitutions can be at the N-terminus, the C-terminus, within the amino acid sequence, or a combination thereof. The one or more substitutions can be contiguous, non-contiguous, or a combination thereof.

### Fusion Polypeptides

Compositions and methods disclosed herein can comprise fusion polypeptides, for example, with a cytokine or cytokine receptor-binding domain joined to a fusion partner, such as a second cytokine or cytokine receptor-binding domain, or an immunoglobulin constant domain.

### E. Fusion Polypeptide Comprising an Engineered IL10 Polypeptide

Compositions and methods disclosed herein can include a fusion polypeptide that comprises an engineered IL10 polypeptide.

In some embodiments, a fusion polypeptide comprises an engineered IL10 polypeptide joined to a second cytokine. Optionally, the engineered IL10 polypeptide can be joined to the second cytokine via a linker, such as a linker disclosed herein.

An N-terminus of the engineered IL10 polypeptide can be joined to a C-terminus of the second cytokine. An N-terminus of the engineered IL10 polypeptide can be joined to a C-terminus of the second cytokine via a linker. An N-terminus of the engineered IL10 polypeptide can be joined to a C-terminus of the second cytokine directly, e.g., without a linker.

An N-terminus of the second cytokine can be joined to a C-terminus of the engineered IL10 polypeptide. An N-terminus of the second cytokine can be joined to a C-terminus of the engineered IL10 polypeptide via a linker. An N-terminus of the second cytokine can be joined to a C-terminus of the engineered IL10 polypeptide directly, e.g., without a linker.

The second cytokine can be, without limitation, IL4, IL13, IL27, IL27A, IL33, TGFβ1, or TGFβ2. The linker can be any linker disclosed herein, e.g., any one of SEQ ID NOs: 22-64 and 187, or a repeat thereof.

For example, any one of SEQ ID NOs: 3-9 can be joined to any one of SEQ ID NOs: 10-21 and 125-139, optionally via a linker. In some embodiments, a C-terminus of any one of SEQ ID NOs: 3-9 is joined to an N-terminus of any one of SEQ ID NOs: 10-21 and 125-139 via a linker comprising any one of SEQ ID NOs: 22-64 or 187, or a repeat thereof. In some embodiments, a C-terminus of any one of SEQ ID NOs: 10-13 is joined to an N-terminus of any one of SEQ ID NOs: 10-21 and 125-139 via a linker comprising any one of SEQ ID NOs: 22-64 or 187, or a repeat thereof.

### 2. Fusion Polypeptide Comprising IL4 and Engineered IL10

In some embodiments, a fusion polypeptide comprises an engineered IL10 polypeptide joined to an IL4 polypeptide. Optionally, the engineered IL10 polypeptide can be joined to the IL4 polypeptide via a linker, such as a linker disclosed herein.

An N-terminus of the engineered IL10 polypeptide can be joined to a C-terminus of the IL4 polypeptide. An N-terminus of the engineered IL10 polypeptide can be joined to a C-terminus of the IL4 polypeptide via a linker. An N-terminus of the engineered IL10 polypeptide can be joined to a C-terminus of the IL4 polypeptide directly, e.g., without a linker.

An N-terminus of the IL4 polypeptide can be joined to a C-terminus of the engineered IL10 polypeptide. An N-terminus of the IL4 polypeptide can be joined to a C-terminus of the engineered IL10 polypeptide via a linker. An N-terminus of the IL4 polypeptide can be joined to a C-terminus of the engineered IL10 polypeptide directly, e.g., without a linker.

For example, any one of SEQ ID NOs: 3-9 can be joined to any one of SEQ ID NOs: 10-13, optionally via a linker. In some embodiments, a C-terminus of any one of SEQ ID NOs: 3-9 is joined to an N-terminus of any one of SEQ ID NOs: 10-13 via a linker comprising any one of SEQ ID NOs: 22-64 and 187, or a repeat thereof. In some embodiments, a C-terminus of any one of SEQ ID NOs: 10-13 is joined to an N-terminus of any one of SEQ ID NOs: 3-9 via a linker comprising any one of SEQ ID NOs: 22-64 and 187, or a repeat thereof.

Illustrative examples of fusion polypeptides comprising an engineered IL10 polypeptide joined to an IL4 polypeptide are provided in SEQ ID NOs: 65-70.

TABLE 6 provides illustrative sequences of fusion polypeptides comprising IL4 and engineered IL10. Linker sequences between the IL4 and engineered IL10 are underlined.

TABLE 6

| SEQ | | |
|---|---|---|
| ID NO: | Description | Sequence |
| 65 | IL4 joined to | KSKAVEQVKNAFNKLQEKGIYKAMSEFDIFINYIEAYMTMKIR |
| | engineered | NGSGGGGSGTSPGQGTQSENSCTHFPGNLPNMLRDLRDAFSRV |
| | IL10 | KTFFQMKDQLDNLLLKESLLEDFKGYLGCQALSEMIQFYLEEV |
| | | MPQAENQDPDIKAHVNSLGENLKTLRLRLRRCHRFLPCENGSG |
| | | GGGSGTHKCDITLQEIIKTLNSLTEQKTLCTELTVTDIFAASKN |
| | | TTEKETFCRAATVLRQFYSHHEKDTRCLGATAQQFHRHKQLIRF |
| | | LKRLDRNLWGLAGLNSCPVKEANQSTLENFLERLKTIMREKYS |
| | | KCSS |
| 66 | IL4 joined to | KSKAVEQVKNAFNKLQEKGIYKAMSEFDIFINYIEAYMTMKIR |
| | engineered | NGSGGGGSGTSPGQGTQSENSCTHFPGNLPNMLRDLRDAFSRV |
| | IL10 with | KTFFQMKDQLDNLLLKESLLEDFKGYLGCQALSEMIQFYLEEV |
| | 6xHis tag | MPQAENQDPDIKAHVNSLGENLKTLRLRLRRCHRFLPCENGSG |
| | | GGGSGTHKCDITLQEIIKTLNSLTEQKTLCTELTVTDIFAASK |
| | | NTTEKETFCRAATVLRQFYSHHEKDTRCLGATAQQFHRHKQLI |
| | | RFLKRLDRNLWGLAGLNSCPVKEANQSTLENFLERLKTIMREK |
| | | YSKCSSHHHHHH |
| 67 | IL4 joined to | HKCDITLQEIIKTLNSLTEQKTLCTELTVTDIFAASKNTTEKET |
| | engineered | FCRAATVLRQFYSHHEKDTRCLGATAQQFHRHKQLIRFLKRLD |
| | IL10 | RNLWGLAGLNSCPVKEANQSTLENFLERLKTIMREKYSKCSSG |
| | | SGGGGSGTSPGQGTQSENSCTHFPGNLPNMLRDLRDAFSRVKT |
| | | FFQMKDQLDNLLLKESLLEDFKGYLGCQALSEMIQFYLEEVMP |
| | | QAENQDPDIKAHVNSLGENLKTLRLRLRRCHRFLPCENGGGSG |
| | | GKSKAVEQVKNAFNKLQEKGIYKAMSEFDIFINYIEAYMTMKI |
| | | RN |
| 68 | IL4 joined to | HHHHHHHKCDITLQEIIKTLNSLTEQKTLCTELTVTDIFAASKN |
| | engineered | TTEKETFCRAATVLRQFYSHHEKDTRCLGATAQQFHRHKQLIR |
| | IL10 with | FLKRLDRNLWGLAGLNSCPVKEANQSTLENFLERLKTIMREKY |
| | 6xHis tag | SKCSSGSGGGGSGTSPGQGTQSENSCTHFPGNLPNMLRDLRDA |
| | | FSRVKTFFQMKDQLDNLLLKESLLEDFKGYLGCQALSEMIQFY |
| | | LEEVMPQAENQDPDIKAHVNSLGENLKTLRLRLRRCHRFLPCE |
| | | NGGGSGGKSKAVEQVKNAFNKLQEKGIYKAMSEFDIFINYIEA |
| | | YMTMKIRN |
| 69 | IL4 joined to | HKCDITLQEIIKTLNSLTEQKTLCTELTVTDIFAASKNTTEKET |
| | engineered | FCRAATVLRQFYSHHEKDTRCLGATAQQFHRHKQLIRFLKRLD |
| | IL10 | RNLWGLAGLNSCPVKEANQSTLENFLERLKTIMREKYSKCSS |
| | | GSGGGGSGTKSKAVEQVKNAFNKLQEKGIYKAMSEFDIFINYI |
| | | EAYMTMKIRNGSGGGGSGTSPGQGTQSENSCTHFPGNLPNML |
| | | RDLRDAFSRVKTFFQMKDQLDNLLLKESLLEDFKGYLGCQAL |
| | | SEMIQFYLEEVMPQAENQDPDIKAHVNSLGENLKTLRLRLRRC |
| | | HRFLPCEN |
| 70 | IL4 joined to | SPGQGTQSENSCTHFPGNLPNMLRDLRDAFSRVKTFFQMKDQ |
| | engineered | LDNLLLKESLLEDFKGYLGCQALSEMIQFYLEEVMPQAENQDP |
| | IL10 | DIKAHVNSLGENLKTLRLRLRRCHRFLPCENGGGSGGKSKAVE |
| | | QVKNAFNKLQEKGIYKAMSEFDIFINYIEAYMTMKIRNGSGGG |
| | | GSGTHKCDITLQEIIKTLNSLTEQKTLCTELTVTDIFAASKNTT |
| | | EKETFCRAATVLRQFYSHHEKDTRCLGATAQQFHRHKQLIRFLK |
| | | RLDRNLWGLAGLNSCPVKEANQSTLENFLERLKTIMREKYSK |
| | | CSS |

### 3. Fusion Polypeptide Comprising IL13 and Engineered IL10

In some embodiments, a fusion polypeptide comprises an engineered IL10 polypeptide joined to an IL13 polypeptide. Optionally, the engineered IL10 polypeptide can be joined to the IL13 polypeptide via a linker, such as a linker disclosed herein.

An N-terminus of the engineered IL10 polypeptide can be joined to a C-terminus of the IL13 polypeptide. An N-terminus of the engineered IL10 polypeptide can be joined to a C-terminus of the IL13 polypeptide via a linker. An N-terminus of the engineered IL10 polypeptide can be joined to a C-terminus of the IL13 polypeptide directly, e.g., without a linker.

An N-terminus of the IL13 polypeptide can be joined to a C-terminus of the engineered IL10 polypeptide. An N-terminus of the IL13 polypeptide can be joined to a C-terminus of the engineered IL10 polypeptide via a linker. An N-terminus of the IL13 polypeptide can be joined to a C-terminus of the engineered IL10 polypeptide directly, e.g., without a linker.

For example, any one of SEQ ID NOs: 3-9 can be joined to any one of SEQ ID NOs: 14-21, optionally via a linker. In some embodiments, a C-terminus of any one of SEQ ID NOs: 3-9 is joined to an N-terminus of any one of SEQ ID NOs: 14-21 via a linker comprising any one of SEQ ID NOs: 22-64 and 187, or a repeat thereof. In some embodiments, a C-terminus of any one of SEQ ID NOs: 14-21 is joined to an N-terminus of any one of SEQ ID NOs: 3-9 via a linker comprising any one of SEQ ID NOs: 22-64 and 187, or a repeat thereof.

Illustrative examples of fusion polypeptides comprising an engineered IL10 polypeptide joined to an IL13 polypeptide are provided in SEQ ID NOs: 71-74.

TABLE 7 provides illustrative sequences of fusion polypeptides comprising IL13 and engineered IL10. Linker sequences between the IL13 and engineered IL10 are underlined.

TABLE 7

| SEQ | | |
|---|---|---|
| ID NO: | Description | Sequence |
| 71 | IL13 joined | SPGPVPPSTALRELIEELVNITQNQKAPLCNGSMVWSINLTAGM |
| | to engineered | YCAALESLINVSGCSAIEKTQRMLSGFCPHKVSAGQFSSLHVR |
| | IL10 | DTKIEVAQFVKDLLLHLKKLFREGRFNGSGGGGSGTKSKAVE |
| | | QVKNAFNKLQEKGIYKAMSEFDIFINYIEAYMTMKIRNGSGGG |
| | | GSGTSPGQGTQSENSCTHFPGNLPNMLRDLRDAFSRVKTFFQM |
| | | KDQLDNLLLKESLLEDFKGYLGCQALSEMIQFYLEEVMPQAE |
| | | NQDPDIKAHVNSLGENLKTLRLRLRRCHRFLPCEN |
| 72 | IL13 joined | KSKAVEQVKNAFNKLQEKGIYKAMSEFDIFINYIEAYMTMKIR |
| | to engineered | NGSGGGGSGTSPGQGTQSENSCTHFPGNLPNMLRDLRDAFSRV |
| | IL10 | KTFFQMKDQLDNLLLKESLLEDFKGYLGCQALSEMIQFYLEEV |
| | | MPQAENQDPDIKAHVNSLGENLKTLRLRLRRCHRFLPCENGSG |
| | | GGGSGTSPGPVPPSTALRELIEELVNITQNQKAPLCNGSMVWSI |
| | | NLTAGMYCAALESLINVSGCSAIEKTQRMLSGFCPHKVSAGQF |
| | | SSLHVRDTKIEVAQFVKDLLLHLKKLFREGREN |
| 73 | IL13 joined | SPGPVPPSTALRELIEELVNITQNQKAPLCNGSMVWSINLTAGM |
| | to engineered | YCAALESLINVSGCSAIEKTQRMLSGFCPHKVSAGQFSSLHVR |
| | IL10 | DTKIEVAQFVKDLLLHLKKLFREGRFNGSGGGGSGTSPGQGTQ |
| | | SENSCTHFPGNLPNMLRDLRDAFSRVKTFFQMKDQLDNLLLK |
| | | ESLLEDFKGYLGCQALSEMIQFYLEEVMPQAENQDPDIKAHVN |
| | | SLGENLKTLRLRLRRCHRFLPCENGGGSGGKSKAVEQVKNAF |
| | | NKLQEKGIYKAMSEFDIFINYIEAYMTMKIRN |
| 74 | IL13 joined | SPGQGTQSENSCTHFPGNLPNMLRDLRDAFSRVKTFFQMKDQ |
| | to engineered | LDNLLLKESLLEDFKGYLGCQALSEMIQFYLEEVMPQAENQDP |
| | IL10 | DIKAHVNSLGENLKTLRLRLRRCHRFLPCENGGGSGGKSKAVE |
| | | QVKNAFNKLQEKGIYKAMSEFDIFINYIEAYMTMKIRNGSGGG |
| | | GSGTSPGPVPPSTALRELIEELVNITQNQKAPLCNGSMVWSINL |
| | | TAGMYCAALESLINVSGCSAIEKTQRMLSGFCPHKVSAGQFSS |
| | | LHVRDTKIEVAQFVKDLLLHLKKLFREGREN |

### F. Engineered Immunocytokines

Compositions and methods disclosed herein can comprise engineered immunocytokines. Engineered immunocytokines disclosed herein can have advantages over the parent cytokines (e.g., over corresponding individual or non-linked cytokines, or over the cytokines present in alternate constructs, such as certain fusion proteins). These advantages can include, for example, a simpler dosing regimen, longer half-life within a subject, and the ability to bind target receptors in close proximity. An engineered immunocytokine can be or can comprise a fusion polypeptide disclosed herein, for example, an engineered immunocytokine can comprise a first fusion polypeptide with an IL4 joined to an immunoglobulin constant domain, and a second fusion polypeptide with an IL10 or engineered IL10 joined to a second immunoglobulin constant domain.

An engineered immunocytokine can be a protein that comprises (i) one or more cytokines or cytokine receptor-binding domains, and (ii) one or more immunoglobulin constant domains. An engineered immunocytokine can be a protein that comprises (i) one or more cytokines or cytokine receptor-binding domains, and (ii) one or more stabilizing domains.

### 4. Cytokines and Cytokine Receptor-Binding Domain

An engineered immunocytokine can comprise one or more cytokine(s) and/or cytokine receptor-binding domain(s).

An engineered immunocytokine can comprise an IL10 receptor binding domain. An engineered immunocytokine can comprise an IL10 polypeptide disclosed herein. An engineered immunocytokine can comprise an engineered IL10 polypeptide disclosed herein.

An engineered immunocytokine can comprise an IL4 receptor binding domain. An engineered immunocytokine can comprise an IL4 polypeptide disclosed herein.

An engineered immunocytokine can comprise an IL13 receptor binding domain. An engineered immunocytokine can comprise an IL13 polypeptide disclosed herein.

An engineered immunocytokine can comprise an IL27 receptor binding domain. An engineered immunocytokine can comprise an IL27, IL27A, or IL27B polypeptide disclosed herein.

An engineered immunocytokine can comprise an IL33 receptor binding domain. An engineered immunocytokine can comprise an IL33 polypeptide disclosed herein.

An engineered immunocytokine can comprise a TGFβ1 receptor binding domain. An engineered immunocytokine can comprise a TGFβ1 polypeptide disclosed herein.

An engineered immunocytokine can comprise a TGFβ2 receptor binding domain. An engineered immunocytokine can comprise a TGFβ2 polypeptide disclosed herein.

An engineered immunocytokine can comprise one or more cytokine receptor binding domains. The engineered immunocytokine can comprise, for example, an IL10 receptor binding domain, an IL4 receptor binding domain, an IL13 receptor binding domain, an IL27 receptor binding domain, an IL33 receptor binding domain, a TGFβ1 receptor binding domain, or a TGFβ2 receptor binding domain. The cytokine receptor binding domain can be an agonistic cytokine receptor binding domain, for example, that induces or activates signaling of the cytokine receptor upon binding. The cytokine receptor binding domain can bind (and, e.g., activate or induce signaling by) a native cytokine receptor disclosed herein, for example, a native IL10 receptor comprising IL-10R1 and IL-10R2 subunits, a native IL4 receptor comprising IL-4Rα and IL-13Rα1 subunits, a native IL4 receptor comprising IL-4Rα and common gamma chain subunits, a native IL13 receptor comprising IL-13Rα1 and IL-4Rα subunits, a native receptor of IL27, a native receptor of IL33, a native receptor of TGFβ1, or a native receptor of TGFβ2. The cytokine receptor binding domain can bind one or more cytokine receptor subunits disclosed herein. The cytokine receptor binding domain can comprise a receptor-binding fragment of a cytokine, for example, a fragment of IL4 that binds to and induces signaling in an IL4 receptor, a fragment of IL10 or engineered IL10 that binds to and induces signaling in an IL10 receptor, a fragment of IL13 that binds to and induces signaling in an IL13 receptor, a fragment of IL27 that binds to and induces signaling in an IL27 receptor, a fragment of IL33 that binds to and induces signaling in an IL33 receptor, a fragment of a fragment of TGFβ1 that binds to and induces signaling in a TGFβ1 receptor, or a fragment of a fragment of TGFβ2 that binds to and induces signaling in a TGFβ2 receptor.

In some embodiments, the cytokine receptor binding domain is a peptide agonist of a cytokine receptor, for example, a peptide agonist of an IL10 receptor, IL4 receptor, IL13 receptor, IL27 receptor, IL33 receptor, TGFβ1 receptor, or TGFβ2 receptor. In some embodiments, the cytokine receptor binding domain is or comprises an antigen-binding fragment of an antibody, e.g., that induces signaling of an IL10 receptor, IL4 receptor, IL13 receptor, IL27 receptor, IL33 receptor, TGFβ1 receptor, or TGFβ2 receptor. An antigen-binding fragment can be, for example, Fab, Fab′, F(ab′)₂, dimers and trimers of Fab conjugates, Fv, scFv, minibodies, dia-, tria-, and tetrabodies, and linear antibodies. In some embodiments, the cytokine receptor binding domain is or comprises an antigen-binding fragment of an ankyrin protein, ankyrin repeat protein, designed ankyrin repeat protein (DARPin), affibody, avimer, adnectin, anticalin, Fynomer, Kunitz domain, knottin, or 0-hairpin mimetic.

An engineered immunocytokine can comprise one or more cytokines, e.g., mammalian or human cytokines. The engineered immunocytokine can comprise, for example, an IL10 polypeptide, engineered IL10 polypeptide, IL4 polypeptide, IL13 polypeptide, IL27 polypeptide, IL27A polypeptide, IL33 polypeptide, TGFβ1 polypeptide, TGFβ2 polypeptide, and/or another cytokine. The cytokine can be a variant, derivative, or functional fragment, for example, that induces or activates signaling by a cytokine receptor.

Functional activity of a cytokine or cytokine receptor-binding domain (e.g., when present in a fusion polypeptide or engineered immunocytokine) can be determined by assessing the activation of intracellular signaling pathways upon incubation of target cells with cytokine or cytokine-receptor-binding domain. The assay can be done in presence or absence of blocking antibody against either the cytokine/cytokine-receptor-binding domain or the cytokine receptor. A reporter cell line can be used to test functional activity, for example, a HEK Blue™ reporter cell line. Additional or alternative functional assays can be used to assess functional activity, for example, a functional assay for IL4 and IL10 is the lipopolysaccharide (LPS) induced TNF release in whole blood (e.g., in presence of anti-IL10 antibody), a functional assay for IL13 is the proliferation of TF1 human erythroleukemic cells, an assay for IL33 function is IL6 production by the mast cell line MC/9, an assay for TGFβ1 or TGFβ2 is inhibition of IL4-dependent growth of mouse T-cell line HT-2, and a functional assay for IL27 can comprise IL6 production by LPS-stimulated THP-1 macrophages.

### 5. Immunoglobulin Constant Domain, Stabilizing Domain

An engineered immunocytokine can comprise an immunoglobulin constant domain and/or a stabilizing domain. Linking a cytokine or cytokine receptor-binding domain to an immunoglobulin constant domain and/or stabilizing domain can facilitate, for example, a simpler dosing regimen and/or longer half-life of the engineered immunocytokine within a subject. A stabilizing domain can be or can comprise any immunoglobulin constant domain disclosed herein. In some embodiments, a stabilizing domain comprises albumin or a fragment or derivative thereof. In some embodiments, a stabilizing domain comprises transferrin, XTEN (genetic fusion of non-exact repeat peptide sequence), CTP (carboxy-terminal peptide), PAS (proline-alanine-serine polymer), ELK (elastin-like peptide), HAP (homo-amino acid polymer), GLK (gelatin-like protein), PEG (polyethylene glycol), or a fragment or derivative thereof.

An immunoglobulin constant domain can be described with reference to the basic four chain antibody unit, which comprises two heavy chain (H) polypeptide sequences and two light chain (L) polypeptide sequences. Each of the heavy chains can comprise one N-terminal variable (VH) domain and three or four C-terminal constant domains (CH1, CH2, and CH3, and in some cases CH4). Each of the light chains can comprise one N-terminal variable (VL) domain and one C-terminal constant (CL) domain. The light chain variable domain is aligned with the heavy chain variable domain and the light chain constant domain is aligned with heavy chain constant domain CH1. Each light chain is linked to a heavy chain by one covalent disulfide bond. The two heavy chains are linked to each other by one or more disulfide bonds depending on the heavy chain isotype. Each heavy and light chain also comprises regularly-spaced intrachain disulfide bridges. The C-terminal constant domains of the heavy chains (e.g., CH2 and CH3, or CH2, CH3, and CH4) comprise the Fc region, Fc domain, or Fc fragment of the antibody, which can mediate effector functions, for example, through interactions with Fc receptors or complement proteins.

The light chain can be designated kappa or lambda based on the amino acid sequence of the constant region. The heavy chain can be designated alpha, delta, epsilon, gamma, or mu based on the amino acid sequence of the constant region. Antibodies can be categorized into five immunoglobulin classes, or isotypes, based on the heavy chain. IgA comprises alpha heavy chains, IgD comprises delta heavy chains, IgE comprises epsilon heavy chains, IgG comprises gamma heavy chains, and IgM comprises mu heavy chains. Antibodies of the IgG, IgD, and IgE classes comprise monomers of the four chain unit described above (two heavy and two light chains), while the IgM and IgA classes can comprise multimers of the four chain unit. The alpha and gamma classes are further divided into subclasses on the basis of differences in the sequence and function of the heavy chain constant region. Subclasses of IgA and IgG expressed by humans include IgG1, IgG2, IgG3, IgG4, IgA1, and IgA2.

Generally the constant regions of an antibody can mediate various effector functions, while the variable regions primarily mediate antigen binding. Different IgG isotypes or subclasses can be associated with different effector functions or therapeutic characteristics, for example, because of interactions with different Fc receptors and/or complement proteins. Fusion polypeptides or engineered immunocytokines comprising constant domains or Fc regions that engage activating Fc receptors can, for example, participate in antibody-dependent cell-mediated cytotoxicity (ADCC), antibody-dependent cellular phagocytosis (ADCP), complement-dependent cytotoxicity (CDC), induction of signaling through immunoreceptor tyrosine-based activation motifs (ITAMs), and induction of cytokine secretion. Fusion polypeptides or engineered immunocytokines comprising Fc regions that engage inhibitory Fc receptors can, for example, induce signaling through immunoreceptor tyrosine-based inhibitory motifs (ITIMs).

Different antibody subclasses comprise varying abilities to elicit immune effector functions, which can be utilized or modified in fusion polypeptides or engineered immunocytokines disclosed herein. For example, wild type IgG1 and IgG3 can effectively recruit complement to activate CDC, and IgG2 elicits minimal ADCC. IgG4 has a lesser ability to trigger immune effector functions and can be used, e.g., where reduced immune effector functions are desired.

Fusion polypeptides and engineered immunocytokines disclosed herein can comprise an immunoglobulin constant domain from a heavy chain and/or light chain of an antibody isotype, class, or subclass disclosed herein. As used herein, “immunoglobulin constant domain” does not necessarily refer to the full constant region of an immunoglobulin chain. Immunoglobulin constant domain can describe at least one domain from the full immunoglobulin constant region. For example, immunoglobulin constant domain can describe a CH1 domain only or a variant, derivative or fragment thereof; a CH2 domain only or a variant, derivative, or fragment thereof; a CH3 domain only or a variant, derivative, or fragment thereof; a CH2 and CH3 domain without CH1; a CH2, CH3, and a hinge or fragment thereof without a CH1 domain; or a CH1, CH2, and CH3, with or without a hinge. In each case, the CH1, CH2, CH3, or hinge, may be a variant, derivative, or fragment thereof. In some embodiments, an immunoglobulin constant domain is a CH2 and CH3, for example of an IgG, IgG1, IgG2, IgG3, IgG4, IgA, IgA1, IgA2, IgM, IgD, or IgE. In some embodiments, an immunoglobulin constant domain is a CH2, CH3, and a hinge or fragment thereof, for example of an IgG, IgG1, IgG2, IgG3, IgG4, IgA, IgA1, IgA2, IgM, IgD, or IgE.

A hinge region of an antibody can be subdivided into three domains, an upper hinge domain, core hinge domain, and lower hinge domain. A core hinge domain can comprise one or more cysteine residues that can form a disulfide bond, for example, with a corresponding core hinge region of a second immunoglobulin constant region. In some embodiments, a hinge or fragment thereof in an immunoglobulin constant domain comprises, consists essentially of, or consists of the upper hinge domain, core hinge domain, and lower hinge domain. In some embodiments, a hinge or fragment thereof in an immunoglobulin constant domain comprises, consists essentially of, or consists of the upper hinge domain. In some embodiments, a hinge or fragment thereof in an immunoglobulin constant domain comprises, consists essentially of, or consists of the core hinge domain. In some embodiments, a hinge or fragment thereof in an immunoglobulin constant domain comprises, consists essentially of, or consists of the lower hinge domain. In some embodiments, a hinge or fragment thereof in an immunoglobulin constant domain comprises, consists essentially of, or consists of the upper hinge domain and core hinge domain. In some embodiments, a hinge or fragment thereof in an immunoglobulin constant domain comprises, consists essentially of, or consists of the upper hinge domain and lower hinge domain. In some embodiments, a hinge or fragment thereof in an immunoglobulin constant domain comprises, consists essentially of, or consists of the core hinge domain and lower hinge domain. In some embodiments, an immunoglobulin constant domain can lack an upper hinge region, for example, to reduce susceptibility to proteolysis. In some embodiments, an upper hinge region can be replaced by a linker disclosed herein, for example, to alter the distance between cytokines or cytokine receptor-binding domains that are present in an engineered immunocytokine, and/or to reduce susceptibility to proteolysis.

In some embodiments, an immunoglobulin constant domain comprises a first hinge domain or hinge fragment from a first immunoglobulin isotype or subclass, and a second hinge domain or hinge fragment from a second immunoglobulin isotype or subclass. For example, in some embodiments an immunoglobulin constant domain comprises a core hinge domain of IgG1 and a lower hinge domain of IgG2, for example, to enhance resistance to proteases and/or reduce effector function.

A fusion polypeptide or engineered immunocytokine can comprise an immunoglobulin constant domain that is a heavy chain constant domain, for example, a CH1, CH2, CH3, and/or CH4 domain, or a variant, derivative, or fragment thereof. The heavy chain constant domain can be a mammalian heavy chain constant domain. The heavy chain constant domain can be a human heavy chain constant domain. In some embodiments, the heavy chain constant domain is a murine, rodent, canine, feline, equine, porcine, primate, or bovine heavy chain constant domain. In some embodiments, the heavy chain constant domain is a non-human heavy chain constant domain.

The immunoglobulin constant domain can be or can comprise a domain from any suitable immunoglobulin isotype, class, or subclass. For example, an immunoglobulin heavy chain constant domain can be a CH1, CH2, and/or CH3 of IgG1 or IgG4 (e.g., mammalian, human, or other IgG1 or IgG4).

A fusion polypeptide or engineered immunocytokine can comprise an immunoglobulin constant domain of IgG, for example, mammalian or human IgG. In some embodiments, a fusion polypeptide or engineered immunocytokine comprises a CH1, hinge, CH2, CH3, fragment thereof, or a combination thereof, of IgG. In some embodiments, a fusion polypeptide or engineered immunocytokine comprises a CH2 and CH3 of IgG. In some embodiments, a fusion polypeptide or engineered immunocytokine comprises a CH2, CH3, and hinge or fragment thereof of IgG. In some embodiments, a fusion polypeptide or engineered immunocytokine comprises an Fc domain of IgG. In some embodiments, a fusion polypeptide or engineered immunocytokine does not include a CH1 domain of IgG. SEQ ID NOs: 75-78 and 146 provide an illustrative sequences of full constant regions of human IgG, which comprises a CH1 domain, hinge, CH2 domain, and CH3 domain. SEQ ID NOs: 84-103 and 147-172 provide illustrative sequences of IgG CH1, CH2, CH3, hinge, and Fc domains. The immunoglobulin constant domain can comprise a modification, e.g., to induce heterodimerization, increase resistance to proteases, alter Fc receptor binding, and/or alter effector function. In some embodiments, an immunoglobulin constant domain comprises a single chain Fc domain, for example, parts of two heavy chains within the one open reading frame.

A fusion polypeptide or engineered immunocytokine can comprise an immunoglobulin constant domain of IgG1, for example, mammalian or human IgG1. In some embodiments, a fusion polypeptide or engineered immunocytokine comprises a CH1, hinge, CH2, CH3, fragment thereof, or a combination thereof, of IgG1. In some embodiments, a fusion polypeptide or engineered immunocytokine comprises a CH2 and CH3 of IgG1. In some embodiments, a fusion polypeptide or engineered immunocytokine comprises a CH2, CH3, and hinge or fragment thereof of IgG1. In some embodiments, a fusion polypeptide or engineered immunocytokine comprises an Fc domain of IgG1. In some embodiments, a fusion polypeptide or engineered immunocytokine does not include a CH1 domain of IgG1. SEQ ID NO: 75 provides an illustrative sequence of a full constant region of human IgG1, which comprises a CH1 domain, hinge, CH2 domain, and CH3 domain. SEQ ID NOs: 84-88 and 147-152 provide illustrative sequences of IgG1 CH1, CH2, CH3, hinge, and Fc domains. In some embodiments, a fusion polypeptide or engineered immunocytokine comprises one or more domains from IgG1 or variants, derivatives, or fragments thereof (e.g., CH2, CH3, and a core hinge domain) and one or more domains from another heavy chain isotype or subclass or variants, derivatives, or fragments thereof (e.g., a lower hinge domain of IgG2, to enhance protease resistance and reduce effector function). The immunoglobulin constant domain can comprise a modification, e.g., to induce heterodimerization, increase resistance to proteases, alter Fc receptor binding, and/or alter effector function.

A fusion polypeptide or engineered immunocytokine can comprise an immunoglobulin constant domain of IgG2, for example, mammalian or human IgG2. In some embodiments, a fusion polypeptide or engineered immunocytokine comprises a CH1, hinge, CH2, CH3, fragment thereof, or a combination thereof, of IgG2. In some embodiments, a fusion polypeptide or engineered immunocytokine comprises a CH2 and CH3 of IgG2. In some embodiments, a fusion polypeptide or engineered immunocytokine comprises a CH2, CH3, and hinge or fragment thereof of IgG2. In some embodiments, a fusion polypeptide or engineered immunocytokine comprises an Fc domain of IgG2. In some embodiments, a fusion polypeptide or engineered immunocytokine does not include a CH1 domain of IgG2. SEQ ID NO: 76 provides an illustrative sequence of a full constant region of human IgG2, which comprises a CH1 domain, hinge, CH2 domain, and CH3 domain. SEQ ID NOs: 89-93 and 153-158 provide illustrative sequences of IgG2 CH1, CH2, CH3, hinge, and Fc domains. The immunoglobulin constant domain can comprise a modification, e.g., to induce heterodimerization, increase resistance to proteases, alter Fc receptor binding, and/or alter effector function.

A fusion polypeptide or engineered immunocytokine can comprise an immunoglobulin constant domain of IgG3, for example, mammalian or human IgG3. In some embodiments, a fusion polypeptide or engineered immunocytokine comprises a CH1, hinge, CH2, CH3, fragment thereof, or a combination thereof, of IgG3. In some embodiments, a fusion polypeptide or engineered immunocytokine comprises CH2 and CH3 of IgG3. In some embodiments, a fusion polypeptide or engineered immunocytokine comprises a CH2, CH3, and hinge or fragment thereof of IgG3. In some embodiments, a fusion polypeptide or engineered immunocytokine comprises an Fc domain of IgG3. In some embodiments, a fusion polypeptide or engineered immunocytokine does not include a CH1 domain of IgG3. SEQ ID NO: 77 provides an illustrative sequence of a full constant region of human IgG3, which comprises a CH1 domain, hinge, CH2 domain, and CH3 domain. SEQ ID NOs: 94-98 and 159-164 provide illustrative sequences of IgG3 CH1, CH2, CH3, hinge, and Fc domains. The immunoglobulin constant domain can comprise a modification, e.g., to induce heterodimerization, increase resistance to proteases, alter Fc receptor binding, and/or alter effector function.

A fusion polypeptide or engineered immunocytokine can comprise an immunoglobulin constant domain of IgG4, for example, mammalian or human IgG4. In some embodiments, a fusion polypeptide or engineered immunocytokine comprises a CH1, hinge, CH2, CH3, fragment thereof, or a combination thereof, of IgG4. In some embodiments, a fusion polypeptide or engineered immunocytokine comprises a CH2 and CH3 of IgG4. In some embodiments, a fusion polypeptide or engineered immunocytokine comprises a CH2, CH3, and hinge or fragment thereof of IgG4. In some embodiments, a fusion polypeptide or engineered immunocytokine comprises an Fc domain of IgG4. In some embodiments, a fusion polypeptide or engineered immunocytokine does not include a CH1 domain of IgG4. SEQ ID NO: 78 and SEQ ID NO: 146 provide illustrative sequences of a full constant region of human IgG4, which comprises a CH1 domain, hinge, CH2 domain, and CH3 domain. SEQ ID NOs: 99-103 and 165-172 provide illustrative sequences of IgG4 CH1, CH2, CH3, hinge, and Fc domains. The immunoglobulin constant domain can comprise a modification, e.g., to induce heterodimerization, increase resistance to proteases, alter Fc receptor binding, and/or alter effector function.

A fusion polypeptide or engineered immunocytokine can comprise an immunoglobulin constant domain of IgA, for example, mammalian or human IgA1 or IgA2. In some embodiments, a fusion polypeptide or engineered immunocytokine comprises a CH1, hinge, CH2, CH3, fragment thereof, or a combination thereof, of IgA. In some embodiments, a fusion polypeptide or engineered immunocytokine comprises CH2 and CH3 of IgA. In some embodiments, a fusion polypeptide or engineered immunocytokine comprises a CH2, CH3, and hinge or fragment thereof of IgA. In some embodiments, a fusion polypeptide or engineered immunocytokine comprises an Fc domain of IgA. In some embodiments, a fusion polypeptide or engineered immunocytokine does not include a CH1 domain of IgA. SEQ ID NO: 80 and SEQ ID NO: 81 provide illustrative sequences of full constant region of human IgA1 and IgA2, respectively, which each comprises a CH1 domain, hinge (e.g., SEQ ID NO: 173 and SEQ ID NO: 174), CH2 domain, and CH3 domain. The immunoglobulin constant domain can comprise a modification, e.g., to induce heterodimerization, increase resistance to proteases, alter Fc receptor binding, and/or alter effector function.

A fusion polypeptide or engineered immunocytokine can comprise an immunoglobulin constant domain of IgE, for example, mammalian or human IgE. In some embodiments, a fusion polypeptide or engineered immunocytokine comprises a CH1, hinge, CH2, CH3, CH4, fragment thereof, or a combination thereof, of IgE. In some embodiments, a fusion polypeptide or engineered immunocytokine comprises CH2, CH3, and CH4 of IgE. In some embodiments, a fusion polypeptide or engineered immunocytokine comprises a CH2, CH3, CH4, and hinge or fragment thereof of IgE. In some embodiments, a fusion polypeptide or engineered immunocytokine comprises an Fc domain of IgE. In some embodiments, a fusion polypeptide or engineered immunocytokine does not include a CH1 domain of IgE. SEQ ID NO: 79 provides an illustrative sequence of a full constant region of human IgE, which comprises a CH1 domain, hinge, CH2 domain, CH3 domain, and CH4 domain. The immunoglobulin constant domain can comprise a modification, e.g., to induce heterodimerization, increase resistance to proteases, alter Fc receptor binding, and/or alter effector function.

A fusion polypeptide or engineered immunocytokine can comprise an immunoglobulin constant domain of IgM, for example, mammalian or human IgM. In some embodiments, a fusion polypeptide or engineered immunocytokine comprises a CH1, hinge, CH2, CH3, CH4, fragment thereof, or a combination thereof, of IgM. In some embodiments, a fusion polypeptide or engineered immunocytokine comprises CH2, CH3, and CH4 of IgM. In some embodiments, a fusion polypeptide or engineered immunocytokine comprises CH2, CH3, CH4, and hinge or fragment thereof of IgM. In some embodiments, a fusion polypeptide or engineered immunocytokine comprises an Fc domain of IgM. In some embodiments, a fusion polypeptide or engineered immunocytokine does not include a CH1 domain of IgM. SEQ ID NO: 82 provides an illustrative sequence of a full constant region of human IgM, which comprises a CH1 domain, hinge, CH2 domain, CH3 domain, and CH4 domain. The immunoglobulin constant domain can comprise a modification, e.g., to induce heterodimerization, increase resistance to proteases, alter Fc receptor binding, and/or alter effector function.

A fusion polypeptide or engineered immunocytokine can comprise an immunoglobulin constant domain of IgD, for example, mammalian or human IgD. In some embodiments, a fusion polypeptide or engineered immunocytokine comprises a CH1, hinge, CH2, CH3, fragment thereof, or a combination thereof, of IgD. In some embodiments, a fusion polypeptide or engineered immunocytokine comprises CH2 and CH3 of IgD. In some embodiments, a fusion polypeptide or engineered immunocytokine comprises CH2, CH3, and hinge or fragment thereof of IgD. In some embodiments, a fusion polypeptide or engineered immunocytokine comprises an Fc domain of IgD. In some embodiments, a fusion polypeptide or engineered immunocytokine does not include a CH1 domain of IgD. SEQ ID NO: 83 provides an illustrative sequence of a full constant region of human IgD, which comprises a CH1 domain, hinge, CH2 domain, and CH3 domain. The immunoglobulin constant domain can comprise a modification, e.g., to induce heterodimerization, increase resistance to proteases, alter Fc receptor binding, and/or alter effector function.

In some embodiments, an engineered immunocytokine or immunoglobulin constant domain comprises a CH2 domain and a CH3 domain. In some embodiments, an engineered immunocytokine or immunoglobulin constant domain comprises a CH2 domain, a CH3 domain, and a CH4 domain. In some embodiments, an engineered immunocytokine or immunoglobulin constant domain lacks a CH1 domain.

In some embodiments, an engineered immunocytokine or immunoglobulin constant domain comprises a CH1 domain and a CH2 domain. In some embodiments, an engineered immunocytokine or immunoglobulin constant domain comprises a CH1 domain and a CH3 domain. In some embodiments, an engineered immunocytokine or immunoglobulin constant domain comprises a CH1 domain and a CH4 domain. In some embodiments, an engineered immunocytokine or immunoglobulin constant domain comprises a CH2 domain and a CH4 domain. In some embodiments, an engineered immunocytokine or immunoglobulin constant domain comprises a CH3 domain and a CH4 domain. In some embodiments, an engineered immunocytokine or immunoglobulin constant domain CH1 domain, CH2 domain, and CH3 domain. In some embodiments, an engineered immunocytokine or immunoglobulin constant domain CH1 domain, CH2 domain, and CH4 domain. In some embodiments, an engineered immunocytokine or immunoglobulin constant domain CH1 domain, CH3 domain, and CH4 domain.

An immunoglobulin constant domain can comprise a light chain constant domain, for example, a CL domain. The light chain constant domain can be a mammalian light chain constant domain. The light chain constant domain can be a human light chain constant domain. In some embodiments, the light chain constant domain is a murine, rodent, canine, feline, equine, porcine, primate, or bovine light chain constant domain. In some embodiments, the light chain constant domain is a non-human light chain constant domain.

The immunoglobulin light chain constant domain can be or can comprise a domain from any suitable immunoglobulin isotype, class, or subclass. For example, an immunoglobulin light chain constant domain can be a lambda (IgL) or kappa (IgK) CL domain (e.g., mammalian, human, or other CL).

A fusion polypeptide or engineered immunocytokine can comprise an immunoglobulin constant domain of IgL or IgK, for example, mammalian or human IgL or IgK. In some embodiments, a fusion polypeptide or engineered immunocytokine comprises a CL of IgL. In some embodiments, a fusion polypeptide or engineered immunocytokine comprises a CL of IgK. SEQ ID NOs: 104 and 105 provide illustrative sequences of IgK and IgL constant domains, respectively.

TABLE 8 provides illustrative sequences of immunoglobulin constant domains that can be used (e.g., in full or in part) or adapted in compositions and methods disclosed herein.

TABLE 8

| SEQ | | |
|---|---|---|
| ID NO: | Description | Sequence |
| 75 | IgG1 | ASTKGPSVFPLAPSSKSTSGGTAALGCLVKDYFPEPVTVSWNS |
| | constant | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPAPI |
| | | EKTISKAKGQPREPQVYTLPPSRDELTKNQVSLTCLVKGFYPSD |
| | | IAVEWESNGQPENNYKTTPPVLDSDGSFFLYSKLTVDKSRWQQ |
| | | GNVFSCSVMHEALHNHYTQKSLSLSPGK |
| 76 | IgG2 | ASTKGPSVFPLAPCSRSTSESTAALGCLVKDYFPEPVTVSWNSG |
| | constant | ALTSGVHTFPAVLQSSGLYSLSSVVTVPSSNFGTQTYTCNVDH |
| | | KPSNTKVDKTVERKCCVECPPCPAPPVAGPSVFLFPPKPKDTL |
| | | MISRTPEVTCVVVDVSHEDPEVQFNWYVDGVEVHNAKTKPRE |
| | | EQFNSTFRVVSVLTVVHQDWLNGKEYKCKVSNKGLPAPIEKTI |
| | | SKTKGQPREPQVYTLPPSREEMTKNQVSLTCLVKGFYPSDISVE |
| | | WESNGQPENNYKTTPPMLDSDGSFFLYSKLTVDKSRWQQGNV |
| | | FSCSVMHEALHNHYTQKSLSLSPGK |
| 77 | IgG3 | ASTKGPSVFPLAPCSRSTSGGTAALGCLVKDYFPEPVTVSWNS |
| | constant | GALTSGVHTFPAVLQSSGLYSLSSVVTVPSSSLGTQTYTCNVN |
| | | HKPSNTKVDKRVELKTPLGDTTHTCPRCPEPKSCDTPPPCPRCP |
| | | EPKSCDTPPPCPRCPEPKSCDTPPPCPRCPAPELLGGPSVFLFP |
| | | PKPKDTLMISRTPEVTCVVVDVSHEDPEVQFKWYVDGVEVHNAK |
| | | TKPREEQYNSTFRVVSVLTVLHQDWLNGKEYKCKVSNKALPA |
| | | PIEKTISKTKGQPREPQVYTLPPSREEMTKNQVSLTCLVKGFYP |
| | | SDIAVEWESSGQPENNYNTTPPMLDSDGSFFLYSKLTVDKSRW |
| | | QQGNIFSCSVMHEALHNRFTQKSLSLSPGK |
| 78 | IgG4 | ASTKGPSVFPLAPCSRSTSESTAALGCLVKDYFPEPVTVSWNSG |
| | constant | ALTSGVHTFPAVLQSSGLYSLSSVVTVPSSSLGTKTYTCNVDH |
| | | KPSNTKVDKRVESKYGPPCPPCPAPEFLGGPSVFLFPPKPKDTL |
| | | MISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIAV |
| | | EWESNGQPENNYKTTPPVLDSDGSFFLYSRLTVDKSRWQEGN |
| | | VFSCSVMHEALHNHYTQKSLSLSLGK |
| 146 | IgG4 | ASTKGPSVFPLAPCSRSTSESTAALGCLVKDYFPEPVTVSWNSG |
| | constant (alt) | ALTSGVHTFPAVLQSSGLYSLSSVVTVPSSSLGTKTYTCNVDH |
| | | KPSNTKVDKRVESKYGPPCPSCPAPEFLGGPSVFLFPPKPKDTL |
| | | MISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIAV |
| | | EWESNGQPENNYKTTPPVLDSDGSFFLYSRLTVDKSRWQEGN |
| | | VFSCSVMHEALHNHYTQKSLSLSLGK |
| 79 | IgE constant | ASTQSPSVFPLTRCCKNIPSNATSVTLGCLATGYFPEPVMVTW |
| | | DTGSLNGTTMTLPATTLTLSGHYATISLLTVSGAWAKQMFTCR |
| | | VAHTPSSTDWVDNKTFSVCSRDFTPPTVKILQSSCDGGGHFPPT |
| | | IQLLCLVSGYTPGTINITWLEDGQVMDVDLSTASTTQEGELAST |
| | | QSELTLSQKHWLSDRTYTCQVTYQGHTFEDSTKKCADSNPRG |
| | | VSAYLSRPSPFDLFIRKSPTITCLVVDLAPSKGTVNLTWSRASG |
| | | KPVNHSTRKEEKQRNGTLTVTSTLPVGTRDWIEGETYQCRVTH |
| | | PHLPRALMRSTTKTSGPRAAPEVYAFATPEWPGSRDKRTLACL |
| | | IQNFMPEDISVQWLHNEVQLPDARHSTTQPRKTKGSGFFVFSR |
| | | LEVTRAEWEQKDEFICRAVHEAASPSQTVQRAVSVNPGK |
| 80 | IgA1 | ASPTSPKVFPLSLCSTQPDGNVVIACLVQGFFPQEPLSVTWSES |
| | constant | GQGVTARNFPPSQDASGDLYTTSSQLTLPATQCLAGKSVTCHV |
| | | KHYTNPSQDVTVPCPVPSTPPTPSPSTPPTPSPSCCHPRLSLHRP |
| | | ALEDLLLGSEANLTCTLTGLRDASGVTFTWTPSSGKSAVQGPP |
| | | ERDLCGCYSVSSVLPGCAEPWNHGKTFTCTAAYPESKTPLTAT |
| | | LSKSGNTFRPEVHLLPPPSEELALNELVTLTCLARGFSPKDVLV |
| | | RWLQGSQELPREKYLTWASRQEPSQGTTTFAVTSILRVAAED |
| | | WKKGDTFSCMVGHEALPLAFTQKTIDRLAGKPTHVNVSVVM |
| | | AEVDGTCY |
| 81 | IgA2 | ASPTSPKVFPLSLDSTPQDGNVVVACLVQGFFPQEPLSVTWSES |
| | constant | GQNVTARNFPPSQDASGDLYTTSSQLTLPATQCPDGKSVTCHV |
| | | KHYTNSSQDVTVPCRVPPPPPCCHPRLSLHRPALEDLLLGSEAN |
| | | LTCTLTGLRDASGATFTWTPSSGKSAVQGPPERDLCGCYSVSS |
| | | VLPGCAQPWNHGETFTCTAAHPELKTPLTANITKSGNTFRPEV |
| | | HLLPPPSEELALNELVTLTCLARGFSPKDVLVRWLQGSQELPRE |
| | | KYLTWASRQEPSQGTTTYAVTSILRVAAEDWKKGETFSCMVG |
| | | HEALPLAFTQKTIDRMAGKPTHINVSVVMAEADGTCY |
| 82 | IgM constant | GSASAPTLFPLVSCENSPSDTSSVAVGCLAQDFLPDSITFSWK |
| | | YKNNSDISSTRGFPSVLRGGKYAATSQVLLPSKDVMQGTDEHV |
| | | VCKVQHPNGNKEKNVPLPVIAELPPKVSVFVPPRDGFFGNPRK |
| | | SKLICQATGFSPRQIQVSWLREGKQVGSGVTTDQVQAEAKESG |
| | | PTTYKVTSTLTIKESDWLGQSMFTCRVDHRGLTFQQNASSMCV |
| | | PDQDTAIRVFAIPPSFASIFLTKSTKLTCLVTDLTTYDSVTISW |
| | | TRONGEAVKTHTNISESHPNATFSAVGEASICEDDWNSGERFT |
| | | CTVTHTDLPSPLKQTISRPKGVALHRPDVYLLPPAREQLNLRES |
| | | ATITCLVTGFSPADVFVQWMQRGQPLSPEKYVTSAPMPEPQAP |
| | | GRYFAHSILTVSEEEWNTGETYTCVVAHEALPNRVTERTVDKS |
| | | TGKPTLYNVSLVMSDTAGTCY |
| 83 | IgD constant | APTKAPDVFPIISGCRHPKDNSPVVLACLITGYHPTSVTVTWYM |
| | | GTQSQPQRTFPEIQRRDSYYMTSSQLSTPLQQWRQGEYKCVVQ |
| | | HTASKSKKEIFRWPESPKAQASSVPTAQPQAEGSLAKATTAPA |
| | | TTRNTGRGGEEKKKEKEKEEQEERETKTPECPSHTQPLGVYLL |
| | | TPAVQDLWLRDKATFTCFVVGSDLKDAHLTWEVAGKVPTGG |
| | | VEEGLLERHSNGSQSQHSRLTLPRSLWNAGTSVTCTLNHPSLPP |
| | | QRLMALREPAAQAPVKLSLNLLASSDPPEAASWLLCEVSGFSP |
| | | PNILLMWLEDQREVNTSGFAPARPPPQPRSTTFWAWSVLRVPA |
| | | PPSPQPATYTCVVSHEDSRTLLNASRSLEVSYVTDHGPMK |
| 84 | IgG1 CH1 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKV |
| 85 | IgG1 CH2 | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAK |
| 147 | IgG1 CH2 | SVFLFPPKPKDTLMISRTPEVTCVVVDVSHEDPEVKFNWYVDG |
| | (alt) | VEVHNAKTKPREEQYNSTYRVVSVLTVLHQDWLNGKEYKCK |
| | | VSNKALPAPIEKTISKAK |
| 86 | IgG1 CH3 | GQPREPQVYTLPPSRDELTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPGK |
| 87 | IgG1 hinge | EPKSCDKTHTCP |
| 148 | IgG1 hinge | EPKSCDKTHTCPPCPAPELLGGP |
| | (alt) | |
| 149 | IgG1 upper | EPKSCDKTHT |
| | hinge | |
| 150 | IgGl core | CPPCP |
| | hinge | |
| 151 | IgGI lower | APELLGGP |
| | hinge | |
| 88 | IgG1 Fc | PCPAPELLGGPSVFLFPPKPKDTLMISRTPEVTCVVVDVSHED |
| | | PEVKFNWYVDGVEVHNAKTKPREEQYNSTYRVVSVLTVLHQD |
| | | WLNGKEYKCKVSNKALPAPIEKTISKAKGQPREPQVYTLPPSR |
| | | DELTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| 152 | IgG1 Fc (alt) | SVFLFPPKPKDTLMISRTPEVTCVVVDVSHEDPEVKFNWYVD |
| | | GVEVHNAKTKPREEQYNSTYRVVSVLTVLHQDWLNGKEYKCK |
| | | VSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPGK |
| 89 | IgG2 CH1 | ASTKGPSVFPLAPCSRSTSESTAALGCLVKDYFPEPVTVSWNSG |
| | | ALTSGVHTFPAVLQSSGLYSLSSVVTVPSSNFGTQTYTCNVDH |
| | | KPSNTKVDKTV |
| 90 | IgG2 CH2 | APPVAGPSVFLFPPKPKDTLMISRTPEVTCVVVDVSHEDPEVQF |
| | | NWYVDGVEVHNAKTKPREEQFNSTFRVVSVLTVVHQDWLNG |
| | | KEYKCKVSNKGLPAPIEKTISKTK |
| 153 | IgG2 CH2 | SVFLFPPKPKDTLMISRTPEVTCVVVDVSHEDPEVQFNWYVDG |
| | (alt) | VEVHNAKTKPREEQFNSTFRVVSVLTVVHQDWLNGKEYKCK |
| | | VSNKGLPAPIEKTISKTK |
| 91 | IgG2 CH3 | GQPREPQVYTLPPSREEMTKNQVSLTCLVKGFYPSDISVEWES |
| | | NGQPENNYKTTPPMLDSDGSFFLYSKLTVDKSRWQQGNVFSC |
| | | SVMHEALHNHYTQKSLSLSPGK |
| 92 | IgG2 hinge | ERKCCVECPPCP |
| 154 | IgG2 hinge | ERKCCVECPPCPAPPVAGP |
| | (alt) | |
| 155 | IgG2 upper | ERK |
| | hinge | |
| 156 | IgG2 core | CCVECPPCP |
| | hinge | |
| 157 | IgG2 lower | APPVAGP |
| | hinge | |
| 93 | IgG2 Fc | APPVAGPSVFLFPPKPKDTLMISRTPEVTCVVVDVSHEDPEVQ |
| | | FNWYVDGVEVHNAKTKPREEQFNSTFRVVSVLTVVHQDWLNG |
| | | KEYKCKVSNKGLPAPIEKTISKTKGQPREPQVYTLPPSREEMTK |
| | | NQVSLTCLVKGFYPSDISVEWESNGQPENNYKTTPPMLDSDGS |
| | | FFLYSKLTVDKSRWQQGNVFSCSVMHEALHNHYTQKSLSLSP |
| | | GK |
| 158 | IgG2 Fc (alt) | SVFLFPPKPKDTLMISRTPEVTCVVVDVSHEDPEVQFNWYVDG |
| | | VEVHNAKTKPREEQFNSTFRVVSVLTVVHQDWLNGKEYKCKV |
| | | SNKGLPAPIEKTISKTKGQPREPQVYTLPPSREEMTKNQVSLT |
| | | CLVKGFYPSDISVEWESNGQPENNYKTTPPMLDSDGSFFLYSK |
| | | LTVDKSRWQQGNVFSCSVMHEALHNHYTQKSLSLSPGK |
| 94 | IgG3 CH1 | ASTKGPSVFPLAPCSRSTSGGTAALGCLVKDYFPEPVTVSWNS |
| | | GALTSGVHTFPAVLQSSGLYSLSSVVTVPSSSLGTQTYTCNVN |
| | | HKPSNTKVDKRV |
| 95 | IgG3 CH2 | APELLGGPSVFLFPPKPKDTLMISRTPEVTCVVVDVSHEDPEVQ |
| | | FKWYVDGVEVHNAKTKPREEQYNSTFRVVSVLTVLHQDWLN |
| | | GKEYKCKVSNKALPAPIEKTISKTK |
| 159 | IgG3 CH2 | SVFLFPPKPKDTLMISRTPEVTCVVVDVSHEDPEVQFKWYVDG |
| | (alt) | VEVHNAKTKPREEQYNSTFRVVSVLTVLHQDWLNGKEYKCK |
| | | VSNKALPAPIEKTISKTK |
| 96 | IgG3 CH3 | GQPREPQVYTLPPSREEMTKNQVSLTCLVKGFYPSDIAVEWES |
| | | SGQPENNYNTTPPMLDSDGSFFLYSKLTVDKSRWQQGNIFSCS |
| | | VMHEALHNRFTQKSLSLSPG |
| 97 | IgG3 hinge | ELKTPLGDTTHTCPRCPEPKSCDTPPPCPRCPEPKSCDTPPPCPR |
| | | CPEPKSCDTPPPCPRCP |
| 160 | IgG3 hinge | ELKTPLGDTTHTCPRCPEPKSCDTPPPCPRCPEPKSCDTPPPCPR |
| | (alt) | CPEPKSCDTPPPCPRCPAPELLGGP |
| 161 | IgG3 upper | ELKTPLGDTTHT |
| | hinge | |
| 162 | IgG3 core | CPRCPEPKSCDTPPPCPRCPEPKSCDTPPPCPRCPEPKSCDTPPPC |
| | hinge | PRCP |
| 163 | IgG3 lower | APELLGGP |
| | hinge | |
| 98 | IgG3 Fc | APELLGGPSVFLFPPKPKDTLMISRTPEVTCVVVDVSHEDPEVQ |
| | | FKWYVDGVEVHNAKTKPREEQYNSTFRVVSVLTVLHQDWLNG |
| | | KEYKCKVSNKALPAPIEKTISKTKGQPREPQVYTLPPSREEMT |
| | | KNQVSLTCLVKGFYPSDIAVEWESSGQPENNYNTTPPMLDSDG |
| | | SFFLYSKLTVDKSRWQQGNIFSCSVMHEALHNRFTQKSLSLSP |
| | | G |
| 164 | IgG3 Fc (alt) | SVFLFPPKPKDTLMISRTPEVTCVVVDVSHEDPEVQFKWYVDG |
| | | VEVHNAKTKPREEQYNSTFRVVSVLTVLHQDWLNGKEYKCK |
| | | VSNKALPAPIEKTISKTKGQPREPQVYTLPPSREEMTKNQVSLT |
| | | CLVKGFYPSDIAVEWESSGQPENNYNTTPPMLDSDGSFFLYSK |
| | | LTVDKSRWQQGNIFSCSVMHEALHNRFTQKSLSLSPG |
| 99 | IgG4 CH1 | ASTKGPSVFPLAPCSRSTSESTAALGCLVKDYFPEPVTVSWNSG |
| | | ALTSGVHTFPAVLQSSGLYSLSSVVTVPSSSLGTKTYTCNVDH |
| | | KPSNTKVDKRV |
| 100 | IgG4 CH2 | APEFLGGPSVFLFPPKPKDTLMISRTPEVTCVVVDVSQEDPEVQ |
| | | FNWYVDGVEVHNAKTKPREEQFNSTYRVVSVLTVLHQDWLN |
| | | GKEYKCKVSNKGLPSSIEKTISKAK |
| 165 | IgG4 CH2 | SVFLFPPKPKDTLMISRTPEVTCVVVDVSQEDPEVQFNWYVDG |
| | (alt) | VEVHNAKTKPREEQFNSTYRVVSVLTVLHQDWLNGKEYKCK |
| | | VSNKGLPSSIEKTISKAK |
| 101 | IgG4 CH3 | GQPREPQVYTLPPSQEEMTKNQVSLTCLVKGFYPSDIAVEWES |
| | | NGQPENNYKTTPPVLDSDGSFFLYSRLTVDKSRWQEGNVFSCS |
| | | VMHEALHNHYTQKSLSLSLGK |
| 102 | IgG4 hinge | ESKYGPPCPSCP |
| 166 | IgG4 hinge | ESKYGPPCPSCPAPEFLGGP |
| | (alt) | |
| 167 | IgG4 hinge | ESKYGPPCPPCP |
| | (alt | |
| 168 | IgG4 hinge | ESKYGPPCPPCPAPEFLGGP |
| | (alt | |
| 169 | IgG4 upper | ESKYGPP |
| | hinge | |
| 170 | IgG4 core | CPSCP |
| | hinge | |
| 171 | IgG4 lower | APEFLGGP |
| | hinge | |
| 103 | IgG4 Fc | APEFLGGPSVFLFPPKPKDTLMISRTPEVTCVVVDVSQEDPEVQ |
| | | FNWYVDGVEVHNAKTKPREEQFNSTYRVVSVLTVLHQDWLN |
| | | GKEYKCKVSNKGLPSSIEKTISKAKGQPREPQVYTLPPSQEEMT |
| | | KNQVSLTCLVKGFYPSDIAVEWESNGQPENNYKTTPPVLDSDG |
| | | SFFLYSRLTVDKSRWQEGNVFSCSVMHEALHNHYTQKSLSLSL |
| | | GK |
| 172 | IgG4 Fc (alt) | SVFLFPPKPKDTLMISRTPEVTCVVVDVSQEDPEVQFNWYVDG |
| | | VEVHNAKTKPREEQFNSTYRVVSVLTVLHQDWLNGKEYKCK |
| | | VSNKGLPSSIEKTISKAKGQPREPQVYTLPPSQEEMTKNQVSLT |
| | | CLVKGFYPSDIAVEWESNGQPENNYKTTPPVLDSDGSFFLYSR |
| | | LTVDKSRWQEGNVFSCSVMHEALHNHYTQKSLSLSLGK |
| 173 | IgA1 hinge | PSTPPTPSPSTPPTPSPS |
| 174 | IgA2 hinge | VPCRVPPPPPCCH |
| 104 | IgK constant | TVAAPSVFIFPPSDEQLKSGTASVVCLLNNFYPREAKVQWKVD |
| | | NALQSGNSQESVTEQDSKDSTYSLSSTLTLSKADYEKHKVYAC |
| | | EVTHQGLSSPVTKSFNRGEC |
| 105 | IgL constant | GQPKANPTVTLFPPSSEELQANKATLVCLISDFYPGAVTVAWK |
| | | ADGSPVKAGVETTKPSKQSNNKYAASSYLSLTPEQWKSHRSY |
| | | SCQVTHEGSTVEKTVAPTECS |

An immunoglobulin constant domain or stabilizing domain disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 75-107 and 146-178. The immunoglobulin constant domain can comprise one or more modifications relative to a disclosed sequence, for example, Fc modifications and/or heterodimerization domain modifications as disclosed herein.

An immunoglobulin constant domain or stabilizing domain disclosed herein can comprise, consist essentially of, or consist of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 75-107 and 146-178.

In some embodiments, an immunoglobulin constant domain or stabilizing domain comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity or sequence similarity to any one of SEQ ID NOs: 75-107 and 146-178.

In some embodiments, the immunoglobulin constant domain or stabilizing domain comprises, consists essentially of, or consists of the amino acid sequence of any one of SEQ ID NOs: 75-107 and 146-178. The immunoglobulin constant domain can comprise one or more modifications relative to a disclosed sequence, for example, Fc modifications and/or heterodimerization domain modifications as disclosed herein.

In some embodiments, the immunoglobulin constant domain or stabilizing domain comprises an amino acid sequence with one or more insertions, deletions, and/or substitutions relative to any one of SEQ ID NOs: 75-107 and 146-178. In some embodiments, the insertions, deletions, and/or substitutions comprise Fc modifications and/or heterodimerization domain modifications as disclosed herein.

For example, the immunoglobulin constant domain or stabilizing domain can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 75-107 and 146-178.

In some embodiments, the immunoglobulin constant domain or stabilizing domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 75-107 and 146-178.

In some embodiments, the immunoglobulin constant domain or stabilizing domain comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 75-107 and 146-178.

The one or more insertions can be at the N-terminus, the C-terminus, within the amino acid sequence, or a combination thereof. The one or more insertions can be contiguous, non-contiguous, or a combination thereof.

In some embodiments, the immunoglobulin constant domain or stabilizing domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 75-107 and 146-178.

In some embodiments, the immunoglobulin constant domain or stabilizing domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 75-107 and 146-178.

In some embodiments, the immunoglobulin constant domain or stabilizing domain comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 75-107 and 146-178.

The one or more deletions can be at the N-terminus, the C-terminus, within the amino acid sequence, or a combination thereof. The one or more deletions can be contiguous, non-contiguous, or a combination thereof.

In some embodiments, the immunoglobulin constant domain or stabilizing domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 75-107 and 146-178. In some embodiments, the substitutions comprise Fc modifications and/or heterodimerization domain modifications as disclosed herein.

In some embodiments, the immunoglobulin constant domain or stabilizing domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 75-107 and 146-178. In some embodiments, the substitutions comprise Fc modifications and/or heterodimerization domain modifications as disclosed herein.

In some embodiments, the immunoglobulin constant domain or stabilizing domain comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 75-107 and 146-178. In some embodiments, the substitutions comprise Fc modifications and/or heterodimerization domain modifications as disclosed herein.

The one or more substitutions can be at the N-terminus, the C-terminus, within the amino acid sequence, or a combination thereof. The one or more substitutions can be contiguous, non-contiguous, or a combination thereof.

### 6. Fc Modifications

Modifications to the constant domains can affect characteristics of an antibody, fusion polypeptide, or engineered immunocytokine that comprises the constant domain, for example, for enhancement or reduction of Fc receptor ligation, enhancement or reduction of ADCC, enhancement or reduction of ADCP, enhancement or reduction of CDC, enhancement or reduction of signaling through ITAMs, enhancement or reduction of cytokine induction, enhancement or reduction of signaling through ITIMs, enhancement or reduction of susceptibility to protease-mediated degradation, or enhancement or reduction of half-life. Modifications can include, for example, amino acid mutations, altering post-translational modifications (e.g., glycosylation), combining domains from different isotypes or subclasses, or a combination thereof. A fusion polypeptide or engineered immunocytokine disclosed herein can comprise an immunoglobulin constant domain or Fc region that is modified to achieve desirable characteristics, for example, reduced binding to one or more particular Fc receptors, reduced induction of immune effector functions, increased resistance to proteases, and enhanced half-life in vivo. Binding to one or more particular Fc receptors can be increased or decreased, while binding to one or more other Fc receptors is not substantially altered. Binding to one or more particular Fc receptors can be increased, while binding to one or more other Fc receptors is not substantially altered. Binding to one or more particular Fc receptors can be decreased, while binding to one or more other Fc receptors is not substantially altered. Binding to one or more particular Fc receptors can be increased, while binding to one or more other Fc receptors is decreased. A particular Fc receptor can be, for example, a single chain IgG receptor (e.g., FcγRIIA, FcγRIIB, FcγRIIC, and FcγRIIIB), IgE receptor (e.g., FcεRII), IgM receptor (e.g., FcμR), or IgA/IgM receptors (e.g., pIgR and FcαμR). A particular Fc receptor can be, for example, a multichain receptor of IgA (e.g., FcαRI), IgE (e.g., FcεRI), or IgG (e.g., FcγRI, FcγRIIIA, FcγRIV, and FcRn). In some embodiments, an immunoglobulin constant domain or Fc region comprises modifications that enhance recycling via the FcRn receptor.

A fusion polypeptide or engineered immunocytokine disclosed herein can comprise an immunoglobulin constant domain or Fc region that is selected or modified to provide suitable characteristics, for example, suitable characteristics for treating a disease or condition as disclosed herein. In some embodiments, IgG1 can be used, for example, to promote immune activation effector functions (e.g., ADCC, ADCP, CDC, ITAM signaling, cytokine induction, or a combination thereof). In some embodiments, IgG4 can be used, for example, in cases where reduced immune effector functions are desirable.

Non-limiting examples of immunoglobulin constant domain modifications and their effects are provided in TABLE 9. The numbering used can be EU numbering. For example, for IgG1, numbering of the constant region according to EU numbering starts with residue number 118, and accordingly, a mutation at residue “L234” in the table below will be at residue L117 in SEQ ID NO: 75, residue “N434” will be residue N317 in SEQ ID NO: 75, etc. Similarly, numbering can be adjusted to the EU numbering of constant region sequences of other isotypes.

TABLE 9

| Effect | Isotype | Mutation(s)/modification(s) |
|---|---|---|
| Enhanced ADCC | IgG1 | F243L/R292P/Y300L/V305I/ |
| | | P396L |
| Enhanced ADCC | IgG1 | S239D/I332E |
| Enhanced ADCC | IgG1 | S239D/I332E/A330L |
| Enhanced ADCC | IgG1 | S298A/E333A/K334A |
| Enhanced ADCC | IgG1 | In one heavy chain: |
| | | L234Y/L235Q/G236W/ |
| | | S239M/H268D/D270E/S298A |
| | | In the opposing heavy chain: |
| | | D270E/K326D/A330M/K334E |
| Enhanced ADCP | IgG1 | G236A/S239D/I332E |
| Enhanced CDC | IgG1 | K326W/E333S |
| Enhanced CDC | IgG1 | S267E/H268F/S324T |
| Enhanced CDC | IgG1, | Combination of domains from |
| | IgG3 | IgG1/IgG3 |
| Enhanced CDC | IgG1 | E345R/E430G/S440Y |
| Loss of glycosylation, | IgG1 | N297A or N297Q or N297G |
| reduced effector functions | | |
| Reduced effector functions | IgG1 | D265A or D265N |
| Reduced effector functions | IgG1, | L235E |
| | IgG4 | |
| Reduced effector functions | IgG1 | L234A/L235A |
| Reduced effector functions | IgG4 | F234A/L235A |
| Reduced effector functions | IgG1 | L234F/L235E/P331S |
| Reduced effector functions | IgG1 | L234A/L235A/P329G |
| Reduced effector functions | IgG4 | F234A/L235A/G237A/P238S |
| Reduced effector functions | IgG4 | F234A/L235A/ΔG236(deletion)/ |
| | | G237A/P238S |
| Reduced effector functions | IgG2, | Combination of domains from |
| | IgG4 | IgG2/IgG4 |
| Reduced effector functions | IgG2 | H268Q/V309L/A330S/P331S |
| Reduced effector functions | IgG2 | V234A/G237A/P238S/H268A/ |
| | | V309L/A330S/P331S |
| Reduced effector functions | IgG1 | L234A/L235A/G237A/P238S/ |
| | | H268A/A330S/P331S |
| Increased half-life | IgG1 | M252Y/T256D |
| Increased half-life | IgG1 | T256D/T307Q |
| Increased half-life | IgG1 | T256D/T307W |
| Increased half-life | IgG1 | M252Y/T256D(or E)/T307Q(or |
| | | W)/N434F(or Y) |
| Increased half-life | IgG1 | M252Y/S254T/T256E |
| Increased half-life | IgG1 | M428L/N434S |
| Increased half-life | IgG3 | R435H |
| Increased half-life | IgG1 | M428L/N434S |
| Increased half-life | IgG1 | T252L/T253S/T254F |
| Increased antigen/Fc | IgG1 | S267E/L328F |
| receptor co-engagement | | |
| Altered antigen/Fc receptor | IgG1 | N325S/L328F |
| co-engagement | | |
| Reduced Fab arm exchange | IgG4 | S228P |
| Reduced effector functions | IgG1 | L234F/L235Q/K322Q/M252Y/ |
| and increased half-life | | S254T/T256E |

### 7. Heterodimerization Domain and Heterodimeric Immunocytokines

Compositions and methods of the disclosure can include heterodimeric immunocytokines, for example, heterodimeric Fc fusions with a first cytokine or cytokine receptor-binding domain appended to a first immunoglobulin constant domain (e.g., Fc chain), and a second cytokine or cytokine receptor-binding domain appended to a second immunoglobulin constant domain (e.g., Fc chain).

Heterodimerization can be induced using a number of methods. Various techniques can be used to promote pairing of desirable heavy chain combinations, rather than random chain associations. Heterodimerization domain(s) can be used to facilitate formation of the heterodimer. A heterodimerization domain can be, for example, an immunoglobulin constant domain or Fc chain with one or more modifications that promote heterodimer formation. A heterodimerization domain can be or can comprise, for example, one or more modifications in an immunoglobulin constant domain or Fc chain that facilitates heterodimer formation.

In some embodiments, engineering strategies are used to introduce mutations into the CH2 and/or CH3 domains to promote heterodimerization based on steric and/or electrostatic complementarity.

Non-limiting examples of heterodimerization domains and/or strategies to induce heterodimerization of polypeptides (e.g., immunoglobulin constant domains disclosed herein, such as Fc domains) include knobs-in-holes, SEEDbody, biochemical optimization and mutations identified therefrom, electrostatic optimization/steering and mutations identified therefrom, DNL (natural association of 2 antibodies or antibody fragments anchored with DDD (dimerization and docking domain) from PKA (protein kinase A) and AD (anchoring domain) from A-kinase anchor protein (AKAP), respectively), CrossMab, LUZ-Y (e.g., leucine zipper tethered at the C-termini of HC and later proteolytically removed, plus point mutation), quadroma (e.g., somatic fusion of hybridomas each encoding a monoclonal antibody), and strand exchange.

In some embodiments, knob-in-hole modifications of immunoglobulin constant (e.g., CH3 or IgG Fc) domains are used to promote formation of heterodimers between the first immunoglobulin constant domain (e.g., Fc chain) and the second immunoglobulin constant domain (e.g., Fc chain). The “knobs in holes” approach allows the generation of complementary interacting interfaces by manipulating key amino acid residues that participate in the Fc dimeric interaction. Amino acids with small side chain are replaced by ones with larger side chains, thereby creating a knob or protrusion in one chain, and vice versa to create a hole or socket in the partner chain.

The “knob” heavy chain can contain a mutation of threonine at a position equivalent to 366 in CH3 of IgG, such as a T366W or T366Y mutation. The “knob” heavy chain can also contain, for example, an F405A mutation.

The “hole” heavy chain can contain multiple mutations, e.g., T366S, L368A, T394W, F405A, and/or Y407V/T). In some embodiments, the “hole” heavy chain comprises T366S, L368A, and Y407V substitutions. In some embodiments, the “hole” heavy chain comprises T366S, L368A, and Y407V substitutions.

The residue numbering can be according to EU numbering (e.g., as described herein).

In some embodiments, an immunoglobulin constant domain (e.g., comprising a knob or hole heterodimerization domain) comprises one or more cysteine replacement residues, for example, to facilitate formation of a disulfide bond with another immunoglobulin constant domain. The immunoglobulin constant domain can comprise a cysteine replacement, for example, at residue Y349, L351, S354, E356, E357, K392, T394, V397, D399, or a combination thereof. A first immunoglobulin constant domain and a second immunoglobulin constant domain can each comprise residues replaced with cysteines, for example, the pair can comprise K392C and D399′C; S354C and Y349′C; E356C and Y349′C; or E357C and Y349′C mutations (where the ′ indicates the mutation is in the second immunoglobulin constant domain). In some embodiments, a first immunoglobulin constant domain comprises an S354C substitution and a second immunoglobulin constant domain comprises a Y349′C substitution. In some embodiments, a first immunoglobulin constant domain comprises a Y349C substitution and a second immunoglobulin constant domain comprises an E356′C substitution.

An immunoglobulin constant domain or a heterodimerization domain can comprise a combination of cysteine replacement residues and knob-in-hole modifications, for example, one or more cysteine replacement residues and one or more knob-in-hole modifications in each of a pair of immunoglobulin constant domains.

In some embodiments, a first immunoglobulin constant domain comprises substitutions at positions S354 and T366, and a second immunoglobulin constant domain comprises substitutions at positions Y349, T366, L368, and Y407. In some embodiments, a first immunoglobulin constant domain comprises S354C and T366W substitutions, and a second immunoglobulin constant domain comprises Y349C, T366S, L368A, and Y407V substitutions. In some embodiments, knob and hole mutations are introduced into the Fc chain as described in or based on the disclosure in Merchant (1998) An efficient route to human bispecific IgG. Nature Biotechnology 16, 677-681.

Illustrative, non-limiting immunoglobulin constant domains (e.g., Fc domains) that comprise heterodimerization domains include SEQ ID NO: 106 (Fc-hole) and SEQ ID NO: 107 (Fc-knob). In some embodiments, similar or corresponding knob and hole mutations are introduced in a different immunoglobulin constant domain or Fc fragment disclosed herein, e.g., an IgG2, IgG3, IgG4, IgA1, or IgA2 Fc fragment, or a CH2 and/or CH3 domain thereof. In some embodiments, other known knobs in holes mutations are used.

In some embodiments, pairs of a heterodimer comprise mutations in CH2 domain residues (e.g., F241R/F243S or F241S/F243R) that remain solvent exposed in aglycosylated IgG molecules to avoid covalent association of knob/knob or hole/hole monomers.

A SEEDbody (Strand-Exchange Engineered Domain) approach can involve creating alternating human IgG and IgA fragments in CH3 to guide heavy chain heterodimerization. For example, patches of IgG and IgA CH3 can be mutually replaced in a heterodimerization domain to facilitate heterodimerization.

In some embodiments, a heterodimerization domain developed using biochemical optimization is used to facilitate heterodimer formation. An illustrative heterodimerization domain comprises mutations in CH3 domains, for example, S364H and F405A in the first chain CH3, and Y349T and T394F in the second chain CH3.

In some embodiments, an electrostatic heterodimerization domain is used to promote heterodimer formation. An engineered immunocytokine can comprise an electrostatic steering modification that favors heterodimeric interaction between a first immunoglobulin constant domain (e.g., first Immunoglobulin heavy chain constant domain) and a second first immunoglobulin constant domain (e.g., second immunoglobulin heavy chain constant domain). For example, charged pair based attraction/repulsion can be tailored in different Fc chains. Mutations in certain residues of the chains can favor heterodimer formation, e.g., (K409D-D399′K, K409D-D399′R, K409E-D399′K, K409E-D399′R, D399′K-E356′K, or K392D-E356′K). In some embodiments, charged amino acids from the core of the CH3 domain are substituted with hydrophobic residues to perturb the structural symmetry, and long-range electrostatic attraction is engineered at the edge of the CH3 domain to promote heterodimer formation. Illustrative examples include a K409W and D399′V/F405′T pair; and K360E and Q347′R pair. In some embodiments, heterodimer formation is promoted by mutations (T350V/L351Y/F405A/Y407V) in a first chain heterodimerization domain and (T350V/T366L/K393L/T394W) in a second chain heterodimerization domain. In some embodiments, heterodimer formation is promoted by mutations (K409D/K392D) in a first chain heterodimerization domain and (D399′K/D357′K) in a second chain heterodimerization domain. In some embodiments, heterodimer formation is promoted by mutations (K409D/K370D) in a first chain heterodimerization domain and (D399′K/D357′K) in a second chain heterodimerization domain. In some embodiments, heterodimer formation is promoted by mutations (K409D/K392D) in a first chain heterodimerization domain and (D339′K/E356′K) in a second chain heterodimerization domain. In some embodiments, heterodimer formation is promoted by mutations (K409D/K392D) in a first chain heterodimerization domain and (D399′K/E356′K) in a second chain heterodimerization domain (e.g., as illustrated in SEQ ID NOs: 175 and 176, respectively).

In some embodiments, an electrostatic heterodimerization domain comprises mutations disclosed in Gunasekaran (2010) Enhancing antibody Fc heterodimer formation through electrostatic steering effects: applications to bispecific molecules and monovalent IgG. J Biol Chem 285: 19637-19646.

In some embodiments, an electrostatic heterodimerization domain comprises substitutions of residues on one CH3 with positively charged (e.g., lysine) residues (e.g., 366 and/or 351), and one or more residues of a second (partner) CH3 are substituted with negatively charged (e.g., glutamic acid or aspartic acid) residues (e.g., 349, 351, 355, and/or 368), for example, as in the Biclonics platform. If light chains are present, they can be common (e.g., the same on both sides of the heterodimer).

In some embodiments, an oxidation-reduction methodology or heterodimerization domain can be used for chain pairing, for example, K409 and L368 can be mutated in the CH3 domain (e.g., of IgG1/IgG2), and the chains can be co-expressed, or purified monomers can be mixed under mild reducing conditions. The K409/L368 mutations can be introduced alone or in combination with IgG1-hinge or IgG2-hinge mutations (if the hinge domain or a fragment thereof is present).

A heterodimerization domain can be generated in a mammalian (e.g., non-human mammalian) immunoglobulin constant domain. For example, in some embodiments a heterodimerization domain is generated in murine IgG as described in Wang et al. (2019) Design and characterization of mouse IgG1 and IgG2a bispecific antibodies for use in syngeneic models. mAbs, 12:1, 1685350. In a non-limiting example, heterodimerization is promoted by generating E356K and D399K mutations in a first murine IgG heavy chain or constant domain therefrom and K409E and K439D in a second murine IgG heavy chain or constant domain therefrom, thereby promoting heterodimerization via electrostatic steering (e.g., as illustrated in SEQ ID NOs: 177 & 178). In some embodiments a non-human mammalian immunoglobulin constant domain is used in a fusion polypeptide or engineered immunocytokine for a therapeutic purpose. In some embodiments a non-human mammalian immunoglobulin constant domain is used in a fusion polypeptide or engineered immunocytokine based on suitability for an experimental model, for example, a mouse model for evaluating pharmacokinetic and/or therapeutic parameters.

TABLE 10 provides illustrative sequences of immunoglobulin constant domains that include heterodimerization domains.

TABLE 10

| SEQ | | |
|---|---|---|
| ID NO: | Description | Sequence |
| 106 | Fc-hole | TCPPCPAPELLGGPSVFLFPPKPKDTLMISRTPEVTCVVVDVS |
| | | HEDPEVKFNWYVDGVEVHNAKTKPREEQYNSTYRVVSVLTVL |
| | | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNHYT |
| | | QKSLSLSPGK |
| 107 | Fc-knob | TCPPCPAPELLGGPSVFLFPPKPKDTLMISRTPEVTCVVVDVS |
| | | HEDPEVKFNWYVDGVEVHNAKTKPREEQYNSTYRVVSVLTVLH |
| | | QDWLNGKEYKCKVSNKALPAPIEKTISKAKGQPREPQVYTLP |
| | | PCRDELTKNQVSLWCLVKGFYPSDIAVEWESNGQPENNYKTT |
| | | PPVLDSDGSFFLYSKLTVDKSRWQQGNVFSCSVMHEALHNHY |
| | | TQKSLSLSPGK |
| 175 | Fc-ES-A | TCPPCPAPEAAGGPSVFLFPPKPKDTLMISRTPEVTCVVVDVS |
| | (K409D/ | HEDPEVKFNWYVDGVEVHNAKTKPREEQYNSTYRVVSVLTVL |
| | K392D; also | HQDWLNGKEYKCKVSNKALPAPIEKTISKAKGQPREPQVYTLP |
| | L234A/ | PSRDELTKNQVSLTCLVKGFYPSDIAVEWESNGQPENNYDTTP |
| | L235A) | PVLDSDGSFFLYSDLTVDKSRWQQGNVFSCSVMHEALHNHYT |
| | | QKSLSLSPGK |
| 176 | Fc-ES-B | TCPPCPAPEAAGGPSVFLFPPKPKDTLMISRTPEVTCVVVDVS |
| | (D399’K/ | HEDPEVKFNWYVDGVEVHNAKTKPREEQYNSTYRVVSVLTVL |
| | E356’K; also | HQDWLNGKEYKCKVSNKALPAPIEKTISKAKGQPREPQVYTLP |
| | L234A/ | PSRDKLTKNQVSLTCLVKGFYPSDIAVEWESNGQPENNYKTTP |
| | L235A) | PVLKSDGSFFLYSKLTVDKSRWQQGNVFSCSVMHEALHNHYT |
| | | QKSLSLSPGK |
| 177 | Murine Fc- | CKPCICTVPEVSSVFIFPPKPKDVLTITLTPKVTCVVVAISKDD |
| | ES-A | PEVQFSWFVDDVEVHTAQTKPREEQINSTFRSVSELPIMHQDWL |
| | (E356K/ | NGKEFKCRVNSAAFPAPIEKTISKTKGRPKAPQVYTIPPPKKQM |
| | D399K) | AKDKVSLTCMITNFFPEDITVEWQWNGQPAENYKNTQPIMKT |
| | | DGSYFVYSKLNVQKSNWEAGNTFTCSVLHEGLHNHHTEKSLS |
| | | HSPGK |
| 178 | Murine Fc- | CKPCICTVPEVSSVFIFPPKPKDVLTITLTPKVTCVVVAISKDD |
| | ES-B | PEVQFSWFVDDVEVHTAQTKPREEQINSTFRSVSELPIMHQDWL |
| | (K439’D/ | NGKEFKCRVNSAAFPAPIEKTISKTKGRPKAPQVYTIPPPKEQM |
| | K409’E) | AKDKVSLTCMITNFFPEDITVEWQWNGQPAENYKNTQPIMDT |
| | | DGSYFVYSELNVQKSNWEAGNTFTCSVLHEGLHNHHTEDSLS |
| | | HSPGK |

An immunoglobulin constant domain (e.g., Fc region) can comprise a combination of modifications to facilitate heterodimerization and other modifications disclosed herein. For example, SEQ ID NOs: 175 and 176 comprise amino acid substitutions to facilitate heterodimerization (K409D/K392D and D399′K/E356′K), and further comprise additional substitutions to reduce effector functions (L234A/L235A: “LALA” mutations). In another example, SEQ ID NOs: 177 and 178 comprise amino acid substitutions to facilitate heterodimerization (E356K/D399K and K439′D/K409′E), and further comprise additional substitutions to reduce Fc receptor binding and effector functions (D265A).

In some embodiments, a first cytokine is fused to a CL domain of an antibody light chain and a second cytokine is fused to a CH1 domain of the antibody heavy chain to provide a heterodimer, which can optionally be coupled to a second heavy chain and second light chain (e.g., with or without the same cytokines appended).

In some embodiments, cytokine and/or immunoglobulin constant domains can be chemically coupled. In some embodiments, COVX-Bodies are generated by chemically linking two pharmacophore peptides via branched azetidinone linker followed by an irreversible site-specific covalent fusion to the scaffold antibody.

In some embodiments, heavy chains can be covalently linked, e.g., via disulfide bonds or click chemistry. In some embodiments, heavy chains are non-covalently associated.

### 8. Illustrative Engineered Immunocytokines

A cytokine or cytokine-receptor-binding domain can be appended to the N or C-terminus of an immunoglobulin constant domain (e.g., Fe arm comprising CH2 and CH3) to generate an engineered immunocytokine or fusion polypeptide.

An IL10 polypeptide, engineered IL10 polypeptide, or IL10 receptor-binding domain disclosed herein can be joined to an immunoglobulin constant domain to generate a fusion polypeptide, engineered cytokine, or component thereof. For example, a C-terminus of an IL10 polypeptide or IL10 receptor-binding domain can be joined to an N-terminus of an immunoglobulin constant domain, optionally via a linker, to generate an engineered immunocytokine or fusion polypeptide. In some embodiments, an N-terminus of an IL10 polypeptide or IL10 receptor-binding domain can be joined to a C-terminus of an immunoglobulin constant domain, optionally via a linker, to generate an engineered immunocytokine or fusion polypeptide.

The IL10 polypeptide or engineered IL10 polypeptide of any one of SEQ ID NOs: 1-9 can be joined to an immunoglobulin constant domain (for example, comprising any one or more SEQ ID NOs: 75-107 and 146-178, or a variant thereof with one or more Fc modifications and/or modifications to induce heterodimerization), optionally via a linker (for example, a linker that comprises the amino acid sequence of any one of SEQ ID NOs: 22-64 and 187, or a repeat thereof). The N-terminus of the IL10 polypeptide or engineered IL10 polypeptide can be joined to the C-terminus of the immunoglobulin constant domain via the linker. The C-terminus of the IL10 polypeptide or engineered IL10 polypeptide can be joined to the N-terminus of the immunoglobulin constant domain via the linker.

In some embodiments, the IL10 polypeptide of SEQ ID NO: 2 is joined to an immunoglobulin constant domain that comprises a CH2, CH3 domain, and heterodimerization domain, via the linker of any one of SEQ ID NOs: 22-64. In some embodiments, a C-terminus of the IL10 polypeptide is joined to an N-terminus of the immunoglobulin constant domain.

In some embodiments, the engineered IL10 polypeptide of SEQ ID NO: 3 is joined to an immunoglobulin constant domain that comprises a CH2, CH3 domain, and heterodimerization domain, via the linker of any one of SEQ ID NOs: 22-64. In some embodiments, a C-terminus of the IL10 polypeptide is joined to an N-terminus of the immunoglobulin constant domain.

In some embodiments, the engineered IL10 polypeptide of SEQ ID NO: 4 is joined to an immunoglobulin constant domain that comprises a CH2, CH3 domain, and heterodimerization domain, via the linker of any one of SEQ ID NOs: 22-64. In some embodiments, a C-terminus of the IL10 polypeptide is joined to an N-terminus of the immunoglobulin constant domain.

In some embodiments, the engineered IL10 polypeptide of SEQ ID NO: 5 is joined to an immunoglobulin constant domain that comprises a CH2, CH3 domain, and heterodimerization domain, via the linker of any one of SEQ ID NOs: 22-64. In some embodiments, a C-terminus of the IL10 polypeptide is joined to an N-terminus of the immunoglobulin constant domain.

In some embodiments, the engineered IL10 polypeptide of SEQ ID NO: 6 is joined to an immunoglobulin constant domain that comprises a CH2, CH3 domain, and heterodimerization domain, via the linker of any one of SEQ ID NOs: 22-64. In some embodiments, a C-terminus of the IL10 polypeptide is joined to an N-terminus of the immunoglobulin constant domain.

In some embodiments, the engineered IL10 polypeptide of SEQ ID NO: 8 is joined to an immunoglobulin constant domain that comprises a CH2, CH3 domain, and heterodimerization domain, via the linker of any one of SEQ ID NOs: 22-64. In some embodiments, a C-terminus of the IL10 polypeptide is joined to an N-terminus of the immunoglobulin constant domain.

In an illustrative example, the C-terminus of the engineered IL10 polypeptide of SEQ ID NO: 3 is joined to the N-terminus of the immunoglobulin constant domain of SEQ ID NO: 107 via the linker of SEQ ID NO: 25, thereby generating the fusion polypeptide or engineered immunocytokine of SEQ ID NO: 110.

In an illustrative example, the C-terminus of the engineered IL10 polypeptide of SEQ ID NO: 8 is joined to the N-terminus of the immunoglobulin constant domain of SEQ ID NO: 107 via the linker of SEQ ID NO: 25, thereby generating the fusion polypeptide or engineered immunocytokine of SEQ ID NO: 111.

In an illustrative example, the C-terminus of the engineered IL10 polypeptide of SEQ ID NO: 3 is joined to the N-terminus of the immunoglobulin constant domain of SEQ ID NO: 106 via the linker of SEQ ID NO: 25, thereby generating the fusion polypeptide or engineered immunocytokine of SEQ ID NO: 116.

In an illustrative example, the N-terminus of the engineered IL10 polypeptide of SEQ ID NO: 3 is joined to the C-terminus of the immunoglobulin constant domain of SEQ ID NO: 107 via the linker of SEQ ID NO: 25, thereby generating the fusion polypeptide or engineered immunocytokine of SEQ ID NO: 116.

In an illustrative example, the C-terminus of the engineered IL10 polypeptide of SEQ ID NO: 8 is joined to the N-terminus of the immunoglobulin constant domain of SEQ ID NO: 106 via the linker of SEQ ID NO: 25, thereby generating the fusion polypeptide or engineered immunocytokine of SEQ ID NO: 118.

In an illustrative example, the C-terminus of the engineered IL10 polypeptide of SEQ ID NO: 8 is joined to the N-terminus of the immunoglobulin constant domain of SEQ ID NO: 175 via the linker of SEQ ID NO: 23, thereby generating the fusion polypeptide or engineered immunocytokine of SEQ ID NO: 194.

An IL4 polypeptide or IL4 receptor-binding domain disclosed herein can be joined to an immunoglobulin constant domain to generate a fusion polypeptide, engineered cytokine, or component thereof. For example, a C-terminus of an IL4 polypeptide or IL4 receptor-binding domain can be joined to an N-terminus of an immunoglobulin constant domain, optionally via a linker, to generate an engineered immunocytokine or fusion polypeptide. In some embodiments, an N-terminus of an IL4 polypeptide or IL4 receptor-binding domain can be joined to a C-terminus of an immunoglobulin constant domain, optionally via a linker, to generate an engineered immunocytokine or fusion polypeptide.

The IL4 polypeptide or engineered IL4 polypeptide of any one of SEQ ID NOs: 10-13 can be joined to an immunoglobulin constant domain (for example, comprising any one or more SEQ ID NOs: 75-107 and 146-178, or a variant thereof with one or more Fc modifications and/or modifications to induce heterodimerization), optionally via a linker (for example, a linker that comprises the amino acid sequence of any one of SEQ ID NOs: 22-64 and 187, or a repeat thereof). The N-terminus of the IL4 polypeptide can be joined to the C-terminus of the immunoglobulin constant domain via the linker. The C-terminus of the IL4 polypeptide can be joined to the N-terminus of the immunoglobulin constant domain via the linker.

In some embodiments, the IL4 polypeptide of SEQ ID NO: 10 is joined to an immunoglobulin constant domain that comprises a CH2, CH3 domain, and heterodimerization domain, via the linker of any one of SEQ ID NOs: 22-64. In some embodiments, a C-terminus of the IL4 polypeptide is joined to an N-terminus of the immunoglobulin constant domain.

In some embodiments, the IL4 polypeptide of SEQ ID NO: 11 is joined to an immunoglobulin constant domain that comprises a CH2, CH3 domain, and heterodimerization domain, via the linker of any one of SEQ ID NOs: 22-64. In some embodiments, a C-terminus of the IL4 polypeptide is joined to an N-terminus of the immunoglobulin constant domain.

In some embodiments, the IL4 polypeptide of SEQ ID NO: 12 is joined to an immunoglobulin constant domain that comprises a CH2, CH3 domain, and heterodimerization domain, via the linker of any one of SEQ ID NOs: 22-64. In some embodiments, a C-terminus of the IL4 polypeptide is joined to an N-terminus of the immunoglobulin constant domain.

In some embodiments, the IL4 polypeptide of SEQ ID NO: 13 is joined to an immunoglobulin constant domain that comprises a CH2, CH3 domain, and heterodimerization domain, via the linker of any one of SEQ ID NOs: 22-64. In some embodiments, a C-terminus of the IL4 polypeptide is joined to an N-terminus of the immunoglobulin constant domain.

In an illustrative example, the C-terminus of the IL4 polypeptide of SEQ ID NO: 10 is joined to the N-terminus of the immunoglobulin constant domain of SEQ ID NO: 106 via the linker of SEQ ID NO: 25, thereby generating the fusion polypeptide or engineered immunocytokine of SEQ ID NO: 108.

In an illustrative example, the N-terminus of the IL4 polypeptide of SEQ ID NO: 10 is joined to the C-terminus of the immunoglobulin constant domain of SEQ ID NO: 106 via the linker of SEQ ID NO: 25, thereby generating the fusion polypeptide or engineered immunocytokine of SEQ ID NO: 112.

In an illustrative example, the C-terminus of the IL4 polypeptide of SEQ ID NO: 10 is joined to the N-terminus of the immunoglobulin constant domain of SEQ ID NO: 107 via the linker of SEQ ID NO: 25, thereby generating the fusion polypeptide or engineered immunocytokine of SEQ ID NO: 113.

An IL13 polypeptide or IL13 receptor-binding domain disclosed herein can be joined to an immunoglobulin constant domain to generate a fusion polypeptide, engineered cytokine, or component thereof. For example, a C-terminus of an IL13 polypeptide or IL13 receptor-binding domain can be joined to an N-terminus of an immunoglobulin constant domain, optionally via a linker, to generate an engineered immunocytokine or fusion polypeptide. In some embodiments, an N-terminus of an IL13 polypeptide or IL13 receptor-binding domain can be joined to a C-terminus of an immunoglobulin constant domain, optionally via a linker, to generate an engineered immunocytokine or fusion polypeptide.

The IL13 polypeptide or engineered IL13 polypeptide of any one of SEQ ID NOs: 14-21 can be joined to an immunoglobulin constant domain (for example, comprising any one or more SEQ ID NOs: 75-107 and 146-178, or a variant thereof with one or more Fc modifications and/or modifications to induce heterodimerization), optionally via a linker (for example, a linker that comprises the amino acid sequence of any one of SEQ ID NOs: 22-64 and 187, or a repeat thereof). The N-terminus of the IL13 polypeptide can be joined to the C-terminus of the immunoglobulin constant domain via the linker. The C-terminus of the IL13 polypeptide can be joined to the N-terminus of the immunoglobulin constant domain via the linker.

In some embodiments, the IL13 polypeptide of SEQ ID NO: 14 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In some embodiments, the IL13 polypeptide of SEQ ID NO: 15 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In some embodiments, the IL13 polypeptide of SEQ ID NO: 16 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In some embodiments, the IL13 polypeptide of SEQ ID NO: 17 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In some embodiments, the IL13 polypeptide of SEQ ID NO: 18 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In some embodiments, the IL13 polypeptide of SEQ ID NO: 19 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In some embodiments, the IL13 polypeptide of SEQ ID NO: 20 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In some embodiments, the IL13 polypeptide of SEQ ID NO: 21 is joined to an immunoglobulin constant domain that comprises a CH2, CH3 domain, and heterodimerization domain, via the linker of any one of SEQ ID NOs: 22-64. In some embodiments, a C-terminus of the IL13 polypeptide is joined to an N-terminus of the immunoglobulin constant domain.

In an illustrative example, the C-terminus of the IL13 polypeptide of SEQ ID NO: 14 is joined to the N-terminus of the immunoglobulin constant domain of SEQ ID NO: 107 via the linker of SEQ ID NO: 23, thereby generating the fusion polypeptide or engineered immunocytokine of SEQ ID NO: 109.

In an illustrative example, the N-terminus of the IL13 polypeptide of SEQ ID NO: 14 is joined to the C-terminus of the immunoglobulin constant domain of SEQ ID NO: 107 via the linker of SEQ ID NO: 23, thereby generating the fusion polypeptide or engineered immunocytokine of SEQ ID NO: 114.

In an illustrative example, the C-terminus of the IL13 polypeptide of SEQ ID NO: 14 is joined to the N-terminus of the immunoglobulin constant domain of SEQ ID NO: 106 via the linker of SEQ ID NO: 23, thereby generating the fusion polypeptide or engineered immunocytokine of SEQ ID NO: 115.

A cytokine or cytokine receptor-binding domain disclosed herein can be joined to an immunoglobulin constant domain to generate a fusion polypeptide, engineered cytokine, or component thereof. For example, a C-terminus of a cytokine or cytokine receptor-binding domain can be joined to an N-terminus of an immunoglobulin constant domain, optionally via a linker, to generate an engineered immunocytokine or fusion polypeptide. In some embodiments, an N-terminus of a cytokine or cytokine receptor-binding domain can be joined to a C-terminus of an immunoglobulin constant domain, optionally via a linker, to generate an engineered immunocytokine or fusion polypeptide.

For example, the cytokine of any one of SEQ ID NOs: 1-21 and 125-139 can be joined to an immunoglobulin constant domain (for example, comprising any one or more SEQ ID NOs: 75-107 and 146-178, or a variant thereof with one or more Fc modifications and/or modifications to induce heterodimerization), optionally via a linker (for example, a linker that comprises the amino acid sequence of any one of SEQ ID NOs: 22-64 and 187, or a repeat thereof). The N-terminus of the cytokine can be joined to the C-terminus of the immunoglobulin constant domain via the linker. The C-terminus of the cytokine can be joined to the N-terminus of the immunoglobulin constant domain via the linker.

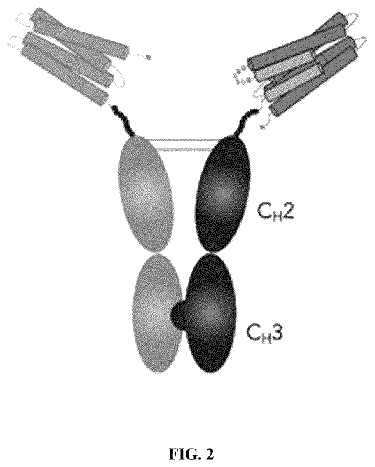

In some embodiments, the disclosure provides an engineered immunocytokine that is heterodimeric, comprising a first polypeptide chain with a first cytokine joined to a first immunoglobulin constant domain, and a second polypeptide chain with a second cytokine joined to a second immunoglobulin constant domain. The first cytokine and the second cytokine can be different. In some embodiments, the first cytokine and the second cytokine are not the same. The first polypeptide chain and the second polypeptide chain can form a covalently-linked heterodimer, for example, via interchain disulfide bonds between the immunoglobulin constant domains. An illustrative schematic of such a heterodimeric immunocytokine with IL4 appended to the N-terminus of a first heavy chain of an Fc fragment and an engineered IL10 appended to the N-terminus of the second heavy chain is provided in FIG. 2.

In some embodiments, the first cytokine and the second cytokine are the same or are variants of the same parent cytokine. In some embodiments, the first cytokine is an IL4 polypeptide, IL10 polypeptide, IL13 polypeptide, IL27 polypeptide, IL27A polypeptide, IL33 polypeptide, TGFβ1 polypeptide, or TGFβ2 polypeptide. In some embodiments, the second cytokine is an IL4 polypeptide, IL10 polypeptide, IL13 polypeptide, IL27 polypeptide, IL27A polypeptide, IL33 polypeptide, TGFβ1 polypeptide, or TGFβ2 polypeptide.

In some embodiments, the first cytokine is an anti-inflammatory cytokine. In some embodiments, the first cytokine is a regulatory cytokine. In some embodiments, the first cytokine is IL4. In some embodiments, the first cytokine is IL10. In some embodiments, the first cytokine is IL13. In some embodiments, the first cytokine is IL27. In some embodiments, the first cytokine is IL27A. In some embodiments, the first cytokine is IL33. In some embodiments, the first cytokine is TGFβ1. In some embodiments, the first cytokine is TGFβ2.

In some embodiments, the second cytokine is an anti-inflammatory cytokine. In some embodiments, the second cytokine is a regulatory cytokine. In some embodiments, the second cytokine is IL4. In some embodiments, the second cytokine is IL10. In some embodiments, the second cytokine is IL13. In some embodiments, the second cytokine is IL27. In some embodiments, the second cytokine is IL27A. In some embodiments, the second cytokine is IL33. In some embodiments, the second cytokine is TGFβ1. In some embodiments, the second cytokine is TGFβ2.

In some embodiments, the first cytokine is not IL4. In some embodiments, the first cytokine is not IL10. In some embodiments, the first cytokine is not IL13. In some embodiments, the first cytokine is not IL27. In some embodiments, the first cytokine is not IL27A. In some embodiments, the first cytokine is not IL33. In some embodiments, the first cytokine is not TGFβ1. In some embodiments, the first cytokine is not TGFβ2.

In some embodiments, the second cytokine is not IL4. In some embodiments, the second cytokine is not IL10. In some embodiments, the second cytokine is not IL13. In some embodiments, the second cytokine is not IL27. In some embodiments, the second cytokine is not IL27A. In some embodiments, the second cytokine is not IL33. In some embodiments, the second cytokine is not TGFβ1. In some embodiments, the second cytokine is not TGFβ2.

For example, a C-terminus of the first cytokine can be joined to an N-terminus of an immunoglobulin constant domain, optionally via a linker, to generate a first fusion polypeptide. In some embodiments, the N-terminus of the first cytokine is joined to the C-terminus of the immunoglobulin constant domain, optionally via a linker, to generate the first fusion polypeptide.

The first cytokine can be joined to an immunoglobulin constant domain (for example, comprising any one or more SEQ ID NOs: 75-107 and 146-178, or a variant thereof with one or more Fc modifications and/or modifications to induce heterodimerization), optionally via a linker (for example, a linker that comprises the amino acid sequence of any one of SEQ ID NOs: 22-64 and 187, or a repeat thereof). The N-terminus of the first cytokine can be joined to the C-terminus of the immunoglobulin constant domain via the linker. The C-terminus of the first cytokine can be joined to the N-terminus of the immunoglobulin constant domain via the linker.

In some embodiments, the first cytokine is joined to an immunoglobulin constant domain that comprises a CH2, CH3 domain, and heterodimerization domain, via the linker of any one of SEQ ID NOs: 22-64 and 187. In some embodiments, a C-terminus of the first cytokine is joined to an N-terminus of the immunoglobulin constant domain.

A C-terminus of the second cytokine can be joined to an N-terminus of an immunoglobulin constant domain, optionally via a linker, to generate a second fusion polypeptide. In some embodiments, the N-terminus of the second cytokine is joined to the C-terminus of the immunoglobulin constant domain, optionally via a linker, to generate the second fusion polypeptide.

The second cytokine can be joined to an immunoglobulin constant domain (for example, comprising any one or more SEQ ID NOs: 75-107 and 146-178, or a variant thereof with one or more Fc modifications and/or modifications to induce heterodimerization), optionally via a linker (for example, a linker that comprises the amino acid sequence of any one of SEQ ID NOs: 22-64 and 187, or a repeat thereof). The N-terminus of the second cytokine can be joined to the C-terminus of the immunoglobulin constant domain via the linker. The C-terminus of the second cytokine can be joined to the N-terminus of the immunoglobulin constant domain via the linker.

In some embodiments, the second cytokine is joined to an immunoglobulin constant domain that comprises a CH2, CH3 domain, and heterodimerization domain, via the linker of any one of SEQ ID NOs: 22-64 and 187. In some embodiments, a C-terminus of the second cytokine is joined to an N-terminus of the immunoglobulin constant domain.

In some embodiments, the first cytokine is joined to the N-terminus of the first immunoglobulin constant domain, and the second cytokine is joined to the N-terminus of the second immunoglobulin constant domain.

In some embodiments, the first cytokine is joined to the C-terminus of the first immunoglobulin constant domain, and the second cytokine is joined to the C-terminus of the second immunoglobulin constant domain.

In some embodiments, the first cytokine is joined to the N-terminus of the first immunoglobulin constant domain, and the second cytokine is joined to the C-terminus of the second immunoglobulin constant domain. In some embodiments, the first cytokine is joined to the C-terminus of the first immunoglobulin constant domain, and the second cytokine is joined to the N-terminus of the second immunoglobulin constant domain.

In some embodiments, the first cytokine is joined to the N-terminus of the first immunoglobulin constant domain and the C-terminus of the first immunoglobulin constant domain (e.g., two copies of the cytokine are present), and/or the second cytokine is joined to the N-terminus of the second immunoglobulin constant domain and the C-terminus of the second immunoglobulin constant domain (e.g., two copies of the cytokine are present).

In some embodiments, the first cytokine is joined to the N-terminus of the first immunoglobulin constant domain and the second cytokine is joined to the C-terminus of the first immunoglobulin constant domain, and/or the first cytokine is joined to the C-terminus of the second immunoglobulin constant domain and the second cytokine is joined to the N-terminus of the second immunoglobulin constant domain.

In some embodiments, the first cytokine is joined to the N-terminus of the first immunoglobulin constant domain and the second cytokine is joined to the C-terminus of the first immunoglobulin constant domain, and/or the first cytokine is joined to the N-terminus of the second immunoglobulin constant domain and the second cytokine is joined to the C-terminus of the second immunoglobulin constant domain.

In some embodiments, the first cytokine is IL4 and the second cytokine is IL10, or vice versa. In some embodiments, the first cytokine is IL4 and the second cytokine is IL13, or vice versa. In some embodiments, the first cytokine is IL4 and the second cytokine is IL27 or IL27A, or vice versa. In some embodiments, the first cytokine is IL4 and the second cytokine is IL33, or vice versa. In some embodiments, the first cytokine is IL4 and the second cytokine is TGFβ1, or vice versa. In some embodiments, the first cytokine is IL4 and the second cytokine is TGFβ2, or vice versa.

In some embodiments, the first cytokine is IL10 and the second cytokine is IL4, or vice versa. In some embodiments, the first cytokine is IL10 and the second cytokine is IL13, or vice versa. In some embodiments, the first cytokine is IL10 and the second cytokine is IL27 or IL27A, or vice versa. In some embodiments, the first cytokine is IL10 and the second cytokine is IL33, or vice versa. In some embodiments, the first cytokine is IL10 and the second cytokine is TGFβ1, or vice versa. In some embodiments, the first cytokine is IL10 and the second cytokine is TGFβ2, or vice versa.

In some embodiments, the first cytokine is IL13 and the second cytokine is IL4, or vice versa. In some embodiments, the first cytokine is IL13 and the second cytokine is IL10, or vice versa. In some embodiments, the first cytokine is IL13 and the second cytokine is IL27 or IL27A, or vice versa. In some embodiments, the first cytokine is IL13 and the second cytokine is IL33, or vice versa. In some embodiments, the first cytokine is IL13 and the second cytokine is TGFβ1, or vice versa. In some embodiments, the first cytokine is IL13 and the second cytokine is TGFβ2, or vice versa.

In some embodiments, the first cytokine is IL27 or IL27A and the second cytokine is IL4, or vice versa. In some embodiments, the first cytokine is IL27 or IL27A and the second cytokine is IL10, or vice versa. In some embodiments, the first cytokine is IL27 or IL27A and the second cytokine is IL13, or vice versa. In some embodiments, the first cytokine is IL27 or IL27A and the second cytokine is IL33, or vice versa. In some embodiments, the first cytokine is IL27 or IL27A and the second cytokine is TGFβ1, or vice versa. In some embodiments, the first cytokine is IL27 or IL27A and the second cytokine is TGFβ2, or vice versa.

In some embodiments, the first cytokine is IL33 and the second cytokine is IL4, or vice versa. In some embodiments, the first cytokine is IL33 and the second cytokine is IL10, or vice versa. In some embodiments, the first cytokine is IL33 and the second cytokine is IL13, or vice versa. In some embodiments, the first cytokine is IL33 and the second cytokine is IL27 or IL27A, or vice versa. In some embodiments, the first cytokine is IL33 and the second cytokine is TGFβ1, or vice versa. In some embodiments, the first cytokine is IL33 and the second cytokine is TGFβ2, or vice versa.

In some embodiments, the first cytokine is TGFβ1 and the second cytokine is IL4, or vice versa. In some embodiments, the first cytokine is TGFβ1 and the second cytokine is IL10, or vice versa. In some embodiments, the first cytokine is TGFβ1 and the second cytokine is IL13, or vice versa. In some embodiments, the first cytokine is TGFβ1 and the second cytokine is IL33, or vice versa. In some embodiments, the first cytokine is TGFβ1 and the second cytokine is IL27 or IL27A, or vice versa. In some embodiments, the first cytokine is TGFβ1 and the second cytokine is TGFβ2, or vice versa.

In some embodiments, the first cytokine is TGFβ2 and the second cytokine is IL4, or vice versa. In some embodiments, the first cytokine is TGFβ2 and the second cytokine is IL10, or vice versa. In some embodiments, the first cytokine is TGFβ2 and the second cytokine is IL13, or vice versa. In some embodiments, the first cytokine is TGFβ2 and the second cytokine is IL33, or vice versa. In some embodiments, the first cytokine is TGFβ2 and the second cytokine is IL27 or IL27A, or vice versa. In some embodiments, the first cytokine is TGFβ2 and the second cytokine is TGFβ2, or vice versa.

Any two suitable fusion polypeptides disclosed herein that comprise a cytokine or cytokine receptor-binding domain and an immunoglobulin constant domain can be combined in heterodimeric immunocytokine.

An illustrative heterodimeric immunocytokine comprises an IL4 polypeptide appended to a first immunoglobulin constant domain, and an IL10 polypeptide or engineered IL10 polypeptide appended to a second immunoglobulin constant domain. Each immunoglobulin constant domain can comprise a CH2, CH3, and a heterodimerization domain (e.g., as individual chains of an Fc fragment with modifications to promote heterodimerization).

In some embodiments, the IL4 and the IL10 are each appended to the N-termini of the respective immunoglobulin constant domains. Illustrative, non-limiting examples include a combination of (i) SEQ ID NO: 108 and SEQ ID NO: 110, (ii) SEQ ID NO: 108 and SEQ ID NO: 111, (iii) SEQ ID NO: 113 and SEQ ID NO: 116, and (iv) SEQ ID NO: 113 and SEQ ID NO: 118.

In some embodiments, the IL4 and the IL10 are each appended to the C-termini of the respective immunoglobulin constant domains. An illustrative example is the combination of SEQ ID NO: 112 and SEQ ID NO: 117.

In some embodiments, the IL4 is appended to the N-terminus of the first immunoglobulin constant domain and the IL10 is appended to the C-terminus of the second immunoglobulin domain. In some embodiments, the IL4 is appended to the C-terminus of the first immunoglobulin constant domain and the IL10 is appended to the N-terminus of the second immunoglobulin domain.

In some embodiments, the IL4 is appended to the N-terminus of the first immunoglobulin constant domain and the C-terminus of the second immunoglobulin constant domain, and the IL10 is appended to the C-terminus of the first immunoglobulin domain and the N-terminus of the second immunoglobulin domain (e.g., two copies of each cytokine are present in the heterodimeric engineered immunocytokine).

An illustrative heterodimeric immunocytokine comprises an IL4 polypeptide appended to a first immunoglobulin constant domain, and an IL13 polypeptide appended to a second immunoglobulin constant domain. Each immunoglobulin constant domain can comprise a CH2, CH3, and a heterodimerization domain (e.g., as individual chains of an Fc fragment with modifications to promote heterodimerization).

In some embodiments, the IL4 and the IL13 are each appended to the N-termini of the respective immunoglobulin constant domains. Illustrative, non-limiting examples include a combination of (i) SEQ ID NO: 108 and SEQ ID NO: 109, and (ii) SEQ ID NO: 113 and SEQ ID NO: 115.

In some embodiments, the IL4 and the IL13 are each appended to the C-termini of the respective immunoglobulin constant domains. An illustrative example is the combination of SEQ ID NO: 112 and SEQ ID NO: 114.

In some embodiments, the IL4 is appended to the N-terminus of the first immunoglobulin constant domain and the IL13 is appended to the C-terminus of the second immunoglobulin domain. In some embodiments, the IL4 is appended to the C-terminus of the first immunoglobulin constant domain and the IL13 is appended to the N-terminus of the second immunoglobulin domain.

In some embodiments, the IL4 is appended to the N-terminus of the first immunoglobulin constant domain and the C-terminus of the second immunoglobulin constant domain, and the IL13 is appended to the C-terminus of the first immunoglobulin domain and the N-terminus of the second immunoglobulin domain (e.g., two copies of each cytokine are present in the heterodimeric engineered immunocytokine).

An illustrative heterodimeric immunocytokine comprises an IL13 polypeptide appended to a first immunoglobulin constant domain, and an IL10 polypeptide or engineered IL10 polypeptide appended to a second immunoglobulin constant domain. Each immunoglobulin constant domain can comprise a CH2, CH3, and a heterodimerization domain (e.g., as individual chains of an Fc fragment with modifications to promote heterodimerization).

In some embodiments, the IL13 and the IL10 are each appended to the N-termini of the respective immunoglobulin constant domains. Illustrative, non-limiting examples include a combination of (i) SEQ ID NO: 109 and SEQ ID NO: 116, (ii) SEQ ID NO: 109 and SEQ ID NO: 118, (iii) SEQ ID NO: 115 and SEQ ID NO: 110, (iv) SEQ ID NO: 115 and SEQ ID NO: 111, and (v) SEQ ID NO: 194 and SEQ ID NO: 180.

In some embodiments, the IL13 and the IL10 are each appended to the C-termini of the respective immunoglobulin constant domains.

In some embodiments, the IL13 is appended to the N-terminus of the first immunoglobulin constant domain and the IL10 is appended to the C-terminus of the second immunoglobulin domain. In some embodiments, the IL13 is appended to the C-terminus of the first immunoglobulin constant domain and the IL10 is appended to the N-terminus of the second immunoglobulin domain.

In some embodiments, the IL13 is appended to the N-terminus of the first immunoglobulin constant domain and the C-terminus of the second immunoglobulin constant domain, and the IL10 is appended to the C-terminus of the first immunoglobulin domain and the N-terminus of the second immunoglobulin domain (e.g., two copies of each cytokine are present in the heterodimeric engineered immunocytokine).

A fusion polypeptide or engineered immunocytokine can comprise a mammalian (e.g., non-human mammalian) cytokine and/or immunoglobulin constant domain. For example, in some embodiments a fusion polypeptide or engineered immunocytokine comprises a murine cytokine and murine immunoglobulin constant domain, as illustrated in SEQ ID NOs: 181, 182, and 195. In some embodiments, a fusion polypeptide or engineered immunocytokine comprises a human cytokine and murine immunoglobulin constant domain, as illustrated in SEQ ID NOs: 183-186. In some embodiments a non-human mammalian immunoglobulin constant domain and/or cytokine is used in a fusion polypeptide or engineered immunocytokine for a therapeutic purpose. In some embodiments a non-human mammalian immunoglobulin constant domain and/or cytokine is used in a fusion polypeptide or engineered immunocytokine based on suitability for an experimental model, for example, a mouse model for evaluating pharmacokinetic and/or therapeutic parameters.

TABLE 11 provides illustrative sequences of fusion polypeptides, engineered immunocytokines, and fusion polypeptides that can be used in engineered immunocytokines. Linker sequences between the cytokine and immunoglobulin constant domain are underlined.

TABLE 11

| SEQ | | |
|---|---|---|
| ID NO: | Description | Sequence |
| 108 | IL4-Fc(hole) | HKCDITLQEIIKTLNSLTEQKTLCTELTVTDIFAASKNTTEKETF |
| | | CRAATVLRQFYSHHEKDTRCLGATAQQFHRHKQLIRFLKRLD |
| | | RNLWGLAGLNSCPVKEANQSTLENFLERLKTIMREKYSKCSSG |
| | | SGGGGSGTCPPCPAPELLGGPSVFLFPPKPKDTLMISRTPEVTC |
| | | VVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVCTLPPSRDELTKNQVSLSCAVKGFYPSDIAVEWESNGQPE |
| | | NNYKTTPPVLDSDGSFFLVSKLTVDKSRWQQGNVFSCSVMHE |
| | | ALHNHYTQKSLSLSPGK |
| 112 | Fc(hole)-IL4 | TCPPCPAPELLGGPSVFLFPPKPKDTLMISRTPEVTCVVVDVS |
| | | HEDPEVKFNWYVDGVEVHNAKTKPREEQYNSTYRVVSVLTVL |
| | | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNHYTQ |
| | | KSLSLSPGKGSGGGGSGHKCDITLQEIIKTLNSLTEQKTLCTEL |
| | | TVTDIFAASKNTTEKETFCRAATVLRQFYSHHEKDTRCLGATA |
| | | QQFHRHKQLIRFLKRLDRNLWGLAGLNSCPVKEANQSTLENFL |
| | | ERLKTIMREKYSKCSS |
| 113 | IL4-Fc(knob) | HKCDITLQEIIKTLNSLTEQKTLCTELTVTDIFAASKNTTEKET |
| | | FCRAATVLRQFYSHHEKDTRCLGATAQQFHRHKQLIRFLKRLD |
| | | RNLWGLAGLNSCPVKEANQSTLENFLERLKTIMREKYSKCSSG |
| | | SGGGGSGTCPPCPAPELLGGPSVFLFPPKPKDTLMISRTPEVTC |
| | | VVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVYTLPPCRDELTKNQVSLWCLVKGFYPSDIAVEWESNGQP |
| | | ENNYKTTPPVLDSDGSFFLYSKLTVDKSRWQQGNVFSCSVMH |
| | | EALHNHYTQKSLSLSPGK |
| 109 | IL13- | SPGPVPPSTALRELIEELVNITQNQKAPLCNGSMVWSINLTAGM |
| | Fc(Knob) | YCAALESLINVSGCSAIEKTQRMLSGFCPHKVSAGQFSSLHVR |
| | | DTKIEVAQFVKDLLLHLKKLFREGRFNTGSGGGGSGTCPPCPA |
| | | PELLGGPSVFLFPPKPKDTLMISRTPEVTCVVVDVSHEDPEVKF |
| | | NWYVDGVEVHNAKTKPREEQYNSTYRVVSVLTVLHQDWLN |
| | | GKEYKCKVSNKALPAPIEKTISKAKGQPREPQVYTLPPCRDELT |
| | | KNQVSLWCLVKGFYPSDIAVEWESNGQPENNYKTTPPVLDSD |
| | | GSFFLYSKLTVDKSRWQQGNVFSCSVMHEALHNHYTQKSLSL |
| | | SPGK |
| 114 | Fc(Knob)- | TCPPCPAPELLGGPSVFLFPPKPKDTLMISRTPEVTCVVVDVSH |
| | IL13 | EDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PCRDELTKNQVSLWCLVKGFYPSDIAVEWESNGQPENNYKTT |
| | | PPVLDSDGSFFLYSKLTVDKSRWQQGNVFSCSVMHEALHNHY |
| | | TQKSLSLSPGKTGSGGGGSGSPGPVPPSTALRELIEELVNITQNQ |
| | | KAPLCNGSMVWSINLTAGMYCAALESLINVSGCSAIEKTQRML |
| | | SGFCPHKVSAGQFSSLHVRDTKIEVAQFVKDLLLHLKKLFREG |
| | | RFN |
| 115 | IL13- | SPGPVPPSTALRELIEELVNITQNQKAPLCNGSMVWSINLTAGM |
| | Fc(hole) | YCAALESLINVSGCSAIEKTQRMLSGFCPHKVSAGQFSSLHVR |
| | | DTKIEVAQFVKDLLLHLKKLFREGRFNTGSGGGGSGTCPPCPA |
| | | PELLGGPSVFLFPPKPKDTLMISRTPEVTCVVVDVSHEDPEVKF |
| | | NWYVDGVEVHNAKTKPREEQYNSTYRVVSVLTVLHQDWLN |
| | | GKEYKCKVSNKALPAPIEKTISKAKGQPREPQVCTLPPSRDELT |
| | | KNQVSLSCAVKGFYPSDIAVEWESNGQPENNYKTTPPVLDSDG |
| | | SFFLVSKLTVDKSRWQQGNVFSCSVMHEALHNHYTQKSLSLS |
| | | PGK |
| 110 | IL10- | KSKAVEQVKNAFNKLQEKGIYKAMSEFDIFINYIEAYMTMKIR |
| | Fc(Knob) | NGSGGGGSGTSPGQGTQSENSCTHFPGNLPNMLRDLRDAFSRV |
| | | KTFFQMKDQLDNLLLKESLLEDFKGYLGCQALSEMIQFYLEEV |
| | | MPQAENQDPDIKAHVNSLGENLKTLRLRLRRCHRFLPCENGSG |
| | | GGGSGTCPPCPAPELLGGPSVFLFPPKPKDTLMISRTPEVTCVV |
| | | VDVSHEDPEVKFNWYVDGVEVHNAKTKPREEQYNSTYRVVS |
| | | VLTVLHQDWLNGKEYKCKVSNKALPAPIEKTISKAKGQPREPQ |
| | | VYTLPPCRDELTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| 111 | IL10- | SPGQGTQSENSCTHFPGNLPNMLRDLRDAFSRVKTFFQMKDQ |
| | Fc(Knob) | LDNLLLKESLLEDFKGYLGCQALSEMIQFYLEEVMPQAENQDP |
| | | DIKAHVNSLGENLKTLRLRLRRCHRFLPCENGGGSGGKSKAVE |
| | | QVKNAFNKLQEKGIYKAMSEFDIFINYIEAYMTMKIRNGSGGG |
| | | GSGTCPPCPAPELLGGPSVFLFPPKPKDTLMISRTPEVTCVVVD |
| | | VSHEDPEVKFNWYVDGVEVHNAKTKPREEQYNSTYRVVSVL |
| | | TVLHQDWLNGKEYKCKVSNKALPAPIEKTISKAKGQPREPQV |
| | | YTLPPCRDELTKNQVSLWCLVKGFYPSDIAVEWESNGQPENN |
| | | YKTTPPVLDSDGSFFLYSKLTVDKSRWQQGNVFSCSVMHEAL |
| | | HNHYTQKSLSLSPGK |
| 116 | IL10- | KSKAVEQVKNAFNKLQEKGIYKAMSEFDIFINYIEAYMTMKIR |
| | Fc(hole) | NGSGGGGSGTSPGQGTQSENSCTHFPGNLPNMLRDLRDAFSRV |
| | | KTFFQMKDQLDNLLLKESLLEDFKGYLGCQALSEMIQFYLEEV |
| | | MPQAENQDPDIKAHVNSLGENLKTLRLRLRRCHRFLPCENGSG |
| | | GGGSGTCPPCPAPELLGGPSVFLFPPKPKDTLMISRTPEVTCVV |
| | | VDVSHEDPEVKFNWYVDGVEVHNAKTKPREEQYNSTYRVVS |
| | | VLTVLHQDWLNGKEYKCKVSNKALPAPIEKTISKAKGQPREPQ |
| | | VCTLPPSRDELTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| 117 | Fc(knob)- | TCPPCPAPELLGGPSVFLFPPKPKDTLMISRTPEVTCVVVDVSH |
| | IL10 | EDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PCRDELTKNQVSLWCLVKGFYPSDIAVEWESNGQPENNYKTT |
| | | PPVLDSDGSFFLYSKLTVDKSRWQQGNVFSCSVMHEALHNHY |
| | | TQKSLSLSPGKGSGGGGSGKSKAVEQVKNAFNKLQEKGIYKA |
| | | MSEFDIFINYIEAYMTMKIRNGSGGGGSGTSPGQGTQSENSCTH |
| | | FPGNLPNMLRDLRDAFSRVKTFFQMKDQLDNLLLKESLLEDFK |
| | | GYLGCQALSEMIQFYLEEVMPQAENQDPDIKAHVNSLGENLK |
| | | TLRLRLRRCHRFLPCEN |
| 118 | IL10- | SPGQGTQSENSCTHFPGNLPNMLRDLRDAFSRVKTFFQMKDQ |
| | Fc(hole) | LDNLLLKESLLEDFKGYLGCQALSEMIQFYLEEVMPQAENQDP |
| | | DIKAHVNSLGENLKTLRLRLRRCHRFLPCENGGGSGGKSKAVE |
| | | QVKNAFNKLQEKGIYKAMSEFDIFINYIEAYMTMKIRNGSGGG |
| | | GSGTCPPCPAPELLGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQV |
| | | CTLPPSRDELTKNQVSLSCAVKGFYPSDIAVEWESNGQPENNY |
| | | KTTPPVLDSDGSFFLVSKLTVDKSRWQQGNVFSCSVMHEALH |
| | | NHYTQKSLSLSPGK |
| 179 | IL4-Fc ES-A | HKCDITLQEIIKTLNSLTEQKTLCTELTVTDIFAASKNTTEKET |
| | (L234A/ | FCRAATVLRQFYSHHEKDTRCLGATAQQFHRHKQLIRFLKRLD |
| | L235A; | RNLWGLAGLNSCPVKEANQSTLENFLERLKTIMREKYSKCSSG |
| | K409D/ | SGGGGSGTCPPCPAPEAAGGPSVFLFPPKPKDTLMISRTPEVT |
| | K392D) | CVVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVYTLPPSRDELTKNQVSLTCLVKGFYPSDIAVEWESNGQPE |
| | | NNYDTTPPVLDSDGSFFLYSDLTVDKSRWQQGNVFSCSVMHE |
| | | ALHNHYTQKSLSLSPGK |
| 194 | IL10M(J)-Fc | SPGQGTQSENSCTHFPGNLPNMLRDLRDAFSRVKTFFQMKDQ |
| | ES-A | LDNLLLKESLLEDFKGYLGCQALSEMIQFYLEEVMPQAENQDP |
| | (L234A/ | DIKAHVNSLGENLKTLRLRLRRCHRFLPCENGGGSGGKSKAVE |
| | L235A; | QVKNAFNKLQEKGIYKAMSEFDIFINYIEAYMTMKIRNTGSGG |
| | K409D/ | GGSGTCPPCPAPEAAGGPSVFLFPPKPKDTLMISRTPEVTCVV |
| | K392D) | VDVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPSRDELTKNQVSLTCLVKGFYPSDIAVEWESNGQPENNY |
| | | DTTPPVLDSDGSFFLYSDLTVDKSRWQQGNVFSCSVMHEALH |
| | | NHYTQKSLSLSPGK |
| 180 | IL13-Fc | SPGPVPPSTALRELIEELVNITQNQKAPLCNGSMVWSINLTAGM |
| | ES-B | YCAALESLINVSGCSAIEKTQRMLSGFCPHKVSAGQFSSLHVR |
| | (L234A/ | DTKIEVAQFVKDLLLHLKKLFREGRFNTGSGGGGSGTCPPCPA |
| | L235A; | PEAAGGPSVFLFPPKPKDTLMISRTPEVTCVVDVDVSHEDPEV |
| | D399'K/ | KFNWYVDGVEVHNAKTKPREEQYNSTYRVVSVLTVLHQDWLN |
| | E356'K) | GKEYKCKVSNKALPAPIEKTISKAKGQPREPQVYTLPPSRDKLT |
| | | KNQVSLTCLVKGFYPSDIAVEWESNGQPENNYKTTPPVLKSDG |
| | | SFFLYSKLTVDKSRWQQGNVFSCSVMHEALHNHYTQKSLSLS |
| | | PGK |
| 181 | Murine IL4- | CDKNHLREIIGILNEVTGEGTPCTEMDVPNVLTATKNTTESELV |
| | Fc-ES-A | CRASKVLRIFYLKHGKTPCLKKNSSVLMELQRLFRAFRCLDSSI |
| | (E356K/ | SCTMNESKSTSLKDFLESLKSIMQMDYSGSGGGGSGTCKPCICT |
| | D399K) | VPEVSSVFIFPPKPKDVLTITLTPKVTCVVVAISKDDPEVQFSW |
| | | FVDDVEVHTAQTKPREEQINSTFRSVSELPIMHQDWLNGKEFK |
| | | CRVNSAAFPAPIEKTISKTKGRPKAPQVYTIPPPKKQMAKDKVS |
| | | LTCMITNFFPEDITVEWQWNGQPAENYKNTQPIMKTDGSYFV |
| | | YSKLNVQKSNWEAGNTFTCSVLHEGLHNHHTEKSLSHSPGK |
| 195 | Alternative | HIHGCDKNHLREIIGILNEVTGEGTPCTEMDVPNVLTATKNTTE |
| | Murine IL4- | SELVCRASKVLRIFYLKHGKTPCLKKNSSVLMELQRLFRAFRC |
| | Fc-ES-A | LDSSISCTMNESKSTSLKDFLESLKSIMQMDYSGSGGGGSGTCK |
| | (E356K/ | PCICTVPEVSSVFIFPPKPKDVLTITLTPKVTCVVVAISKDDPE |
| | D399K) | VQFSWFVDDVEVHTAQTKPREEQINSTFRSVSELPIMHQDWLNG |
| | | KEFKCRVNSAAFPAPIEKTISKTKGRPKAPQVYTIPPPKKQMAK |
| | | DKVSLTCMITNFFPEDITVEWQWNGQPAENYKNTQPIMKTDG |
| | | SYFVYSKLNVQKSNWEAGNTFTCSVLHEGLHNHHTEKSLSHS |
| | | PGK |
| 182 | Murine IL 13 | APGPVPRSVSLPLTLKELIEELSNITQDQTPLCNGSMVWSVDLA |
| | Fc-ES-B | AGGFCVALDSLTNISNCNAIYRTQRILHGLCNRKAPTTVSSLPD |
| | (K439D/ | TKIEVAHFITKLLSYTKQLFRHGPFTGSGGGGSGTCKPCICTVP |
| | K409E) | EVSSVFIFPPKPKDVLTITLTPKVTCVVVAISKDDPEVQFSWFV |
| | | DDVEVHTAQTKPREEQINSTFRSVSELPIMHQDWLNGKEFKCRV |
| | | NSAAFPAPIEKTISKTKGRPKAPQVYTIPPPKEQMAKDKVSLTC |
| | | MITNFFPEDITVEWQWNGQPAENYKNTQPIMDTDGSYFVYSEL |
| | | NVQKSNWEAGNTFTCSVLHEGLHNHHTEDSLSHSPGK |
| 183 | hIL4-mFc- | HKCDITLQEIIKTLNSLTEQKTLCTELTVTDIFAASKNTTEKET |
| | ES-A | FCRAATVLRQFYSHHEKDTRCLGATAQQFHRHKQLIRFLKRLD |
| | (E356K/ | RNLWGLAGLNSCPVKEANQSTLENFLERLKTIMREKYSKCSSGS |
| | D399K) | GGGGSGTCKPCICTVPEVSSVFIFPPKPKDVLTITLTPKVTCVV |
| | | VAISKDDPEVQFSWFVDDVEVHTAQTKPREEQINSTFRSVSELP |
| | | IMHQDWLNGKEFKCRVNSAAFPAPIEKTISKTKGRPKAPQVYT |
| | | IPPPKKQMAKDKVSLTCMITNFFPEDITVEWQWNGQPAENYK |
| | | NTQPIMKTDGSYFVYSKLNVQKSNWEAGNTFTCSVLHEGLHN |
| | | HHTEKSLSHSPGK |
| 184 | hIL13-mFc- | SPGPVPPSTALRELIEELVNITQNQKAPLCNGSMVWSINLTAGM |
| | ES-B | YCAALESLINVSGCSAIEKTQRMLSGFCPHKVSAGQFSSLHVR |
| | (K439D/ | DTKIEVAQFVKDLLLHLKKLFREGRFNTGSGGGGSGTCKPCIC |
| | K409E) | TVPEVSSVFIFPPKPKDVLTITLTPKVTCVVVAISKDDPEVQFS |
| | | WFVDDVEVHTAQTKPREEQINSTFRSVSELPIMHQDWLNGKEFK |
| | | CRVNSAAFPAPIEKTISKTKGRPKAPQVYTIPPPKEQMAKDKVS |
| | | LTCMITNFFPEDITVEWQWNGQPAENYKNTQPIMDTDGSYFV |
| | | YSELNVQKSNWEAGNTFTCSVLHEGLHNHHTEDSLSHSPGK |
| 185 | hIL 10(J)- | SPGQGTQSENSCTHFPGNLPNMLRDLRDAFSRVKTFFQMKDQ |
| | mFc-ES-B | LDNLLLKESLLEDFKGYLGCQALSEMIQFYLEEVMPQAENQDP |
| | (K439D/ | DIKAHVNSLGENLKTLRLRLRRCHRFLPCENGGGSGGKSKAVE |
| | K409E) | QVKNAFNKLQEKGIYKAMSEFDIFINYIEAYMTMKIRNTGSGG |
| | | GGSGTCKPCICTVPEVSSVFIFPPKPKDVLTITLTPKVTCVVVA |
| | | ISKDDPEVQFSWFVDDVEVHTAQTKPREEQINSTFRSVSELPIM |
| | | HQDWLNGKEFKCRVNSAAFPAPIEKTISKTKGRPKAPQVYTIPP |
| | | PKEQMAKDKVSLTCMITNFFPEDITVEWQWNGQPAENYKNTQ |
| | | PIMDTDGSYFVYSELNVQKSNWEAGNTFTCSVLHEGLHNHHT |
| | | EDSLSHSPGK |
| 186 | hIL 10(S)- | KSKAVEQVKNAFNKLQEKGIYKAMSEFDIFINYIEAYMTMKIR |
| | mFc-ES-B | NGSGGGGSGTSPGQGTQSENSCTHFPGNLPNMLRDLRDAFSRV |
| | (K439D/ | KTFFQMKDQLDNLLLKESLLEDFKGYLGCQALSEMIQFYLEEV |
| | K409E) | MPQAENQDPDIKAHVNSLGENLKTLRLRLRRCHRFLPCENTGSG |
| | | GGGSGTCKPCICTVPEVSSVFIFPPKPKDVLTITLTPKVTCVVV |
| | | AISKDDPEVQFSWFVDDVEVHTAQTKPREEQINSTFRSVSELPI |
| | | MHQDWLNGKEFKCRVNSAAFPAPIEKTISKTKGRPKAPQVYTI |
| | | PPPKEQMAKDKVSLTCMITNFFPEDITVEWQWNGQPAENYKN |
| | | TQPIMDTDGSYFVYSELNVQKSNWEAGNTFTCSVLHEGLHNH |
| | | HTEDSLSHSPGK |

A fusion polypeptide or engineered immunocytokine disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of SEQ ID NOs: 65-74, 108-118, 179-186, 194, and 195. The fusion polypeptide or engineered immunocytokine can comprise one or more modifications relative to a disclosed sequence, for example, Fc modifications and/or heterodimerization domain modifications as disclosed herein.

A fusion polypeptide or engineered immunocytokine disclosed herein can comprise, consist essentially of, or consist of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of SEQ ID NOs: 65-74, 108-118, 179-186, 194, and 195.

In some embodiments, a fusion polypeptide or engineered immunocytokine comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, or about 99.5% or about 100% sequence identity or sequence similarity to any one of SEQ ID NOs: 65-74, 108-118, 179-186, 194, and 195.

In some embodiments, the fusion polypeptide or engineered immunocytokine comprises, consists essentially of, or consists of the amino acid sequence of any one of SEQ ID NOs: 65-74, 108-118, 179-186, 194, and 195.

In some embodiments, the fusion polypeptide or engineered immunocytokine comprises an amino acid sequence with one or more insertions, deletions, and/or substitutions relative to any one of SEQ ID NOs: 65-74, 108-118, 179-186, 194, and 195. The insertions, deletions, and/or substitutions can comprise one or more modifications relative to a disclosed sequence, for example, Fc modifications and/or heterodimerization domain modifications as disclosed herein.

For example, the fusion polypeptide or engineered immunocytokine can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 65-74, 108-118, 179-186, 194, and 195.

In some embodiments, the fusion polypeptide or engineered immunocytokine comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 65-74, 108-118, 179-186, 194, and 195.

In some embodiments, the fusion polypeptide or engineered immunocytokine comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 65-74, 108-118, 179-186, 194, and 195.

The one or more insertions can be at the N-terminus, the C-terminus, within the amino acid sequence, or a combination thereof. The one or more insertions can be contiguous, non-contiguous, or a combination thereof.

In some embodiments, the fusion polypeptide or engineered immunocytokine comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 65-74, 108-118, 179-186, 194, and 195.

In some embodiments, the fusion polypeptide or engineered immunocytokine comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 65-74, 108-118, 179-186, 194, and 195.

In some embodiments, the fusion polypeptide or engineered immunocytokine comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 65-74, 108-118, 179-186, 194, and 195.

The one or more deletions can be at the N-terminus, the C-terminus, within the amino acid sequence, or a combination thereof. The one or more deletions can be contiguous, non-contiguous, or a combination thereof.

In some embodiments, the fusion polypeptide or engineered immunocytokine comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 65-74, 108-118, 179-186, 194, and 195.

In some embodiments, the fusion polypeptide or engineered immunocytokine comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 65-74, 108-118, 179-186, 194, and 195.

In some embodiments, the fusion polypeptide or engineered immunocytokine comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 65-74, 108-118, 179-186, 194, and 195.

The one or more substitutions can be at the N-terminus, the C-terminus, within the amino acid sequence, or a combination thereof. The one or more substitutions can be contiguous, non-contiguous, or a combination thereof.

The degree of sequence identity between two sequences can be determined, for example, by comparing the two sequences using computer programs designed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A global alignment algorithm, such as a Needleman and Wunsch algorithm, can be used to align two sequences over their entire length, maximizing the number of matches and minimizes the number of gaps. Default settings can be used.

To generate similarity scores for two amino acid sequences, scoring matrices can be used that assign positive scores for some non-identical amino acids (e.g., amino acids with similar physio-chemical properties and/or amino acids that exhibit frequent substitutions in orthologs, homologs, or paralogs), Non-limiting examples of scoring matrices include PAM30, PAM70, PAM250, BLOSUM45, BLOSUM50, BLOUM62, BLOSUM80, and BLOSUM90.

Amino acids can include genetically encoded and non-genetically encoded occurring amino acids. Amino acids can include naturally occurring and non-naturally occurring amino acids. Amino acids can be L forms or D forms. Substitutions disclosed herein can include conservative and/or non-conservative amino acid substitutions. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser (S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser (S) and Thr (T). A conservative amino acid change can be an amino acid change from one nonpolar amino acid to another nonpolar amino acid. Nonpolar amino acids can include Leu (L), Val (V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

A protein or polypeptide disclosed herein can comprise an N-terminal methionine. A protein or polypeptide disclosed herein can lack an N-terminal methionine.

Polypeptides disclosed herein can comprise chemical modifications, such as glycosylation, fucosylation, sialylation, and/or pegylation.

### G. Linkers and Tags

A polypeptide disclosed herein, such as an engineered IL10 polypeptide, fusion polypeptide, or engineered immunocytokine, can comprise one or more linkers for example, between different domains of the polypeptide. A linker can be a chemical bond, for example, a covalent bond or a non-covalent bond. A linker as described herein can include a flexible or rigid linker. A linker can be a peptide.

A linker can be selected to achieve a desired functionality of the polypeptide. For example, various linkers can be tested to identify a configuration of one or more linkers that allow an engineered IL10 polypeptide, fusion polypeptide, or engineered immunocytokine to induce a desirable profile of cytokine receptor clustering, cytokine receptor signaling, in vitro activity, and/or in vivo activity. In some embodiments, a fusion polypeptide or engineered immunocytokine is configured with linkers appropriate to induce a desirable profile of clustering of an IL4 receptor and IL10 receptor, an IL4 receptor and IL13 receptor, or an IL10 receptor and IL13 receptor. In some embodiments, a fusion polypeptide or engineered immunocytokine is configured with linkers appropriate to induce a desirable signaling profile of IL4 receptor and IL10 receptor, IL4 receptor and IL13 receptor, or IL10 receptor and IL13 receptor, and the desirable signaling profile can facilitate surprising therapeutic activity of the fusion polypeptide or engineered immunocytokine in vitro and/or in vivo.

A linker can comprise a linker sequence, for example, a linker peptide sequence. The length a linker can be adjusted to allow for proper folding or to increase or decrease biological activity of the engineered IL10 polypeptide, fusion polypeptide, or engineered immunocytokine. A linker can be, for example, about 1, about 2, about 3, about 4, about 5, about 6, about 7, about 8, about 9, about 10, about 11, about 12, about 13, about 14, about 15, about 20, about 25, about 30, about 40, about 50, about 60, or about 70 amino acid residues in length. In some cases, a linker can be, for example at least about 2, at least about 3, at least about 4, at least about 5, at least about 6, at least about 7, at least about 8, at least about 9, at least about 10, at least about 15, at least about 20, at least about 30, at least about 40, or at least about 50 amino acids in length. In some cases, a linker can be, for example at most about 2, at most about 3, at most about 4, at most about 5, at most about 6, at most about 7, at most about 8, at most about 9, at most about 10, at most about 15, at most about 20, at most about 30, at most about 40, at most about 50, at most about 60, at most about 70, at most about 80, or at most about 100 amino acids in length. In some cases, a linker is 5-20 amino acids in length. In some cases, a linker is 10-20 amino acids in length.

A flexible linker can have a sequence containing glycine residues. The small size of the glycine residues can provide flexibility, and allow for mobility of the connected protein domains. The incorporation of serine or threonine can maintain the stability of the linker in aqueous conditions by forming hydrogen bonds with the water molecules, thereby reducing unfavorable interactions between the linker and protein moieties. In some cases flexible linkers can also contain additional amino acids, such as threonine and alanine, to maintain flexibility, and/or polar amino acids such as lysine and glutamine, to improve solubility.

A rigid linker can have, for example, an alpha helix-structure. An alpha-helical rigid linker can act as a spacer between protein domains. A rigid linker can have a proline-rich sequence, (XP)n, with X designating alanine, lysine, glutamine, or any amino acid, and n designating a number of repeats. The presence of proline in non-helical linkers can increase stiffness, and allow for effective separation of protein domains.

In some embodiments, a linker used in a polypeptide disclosed herein is a stiff linker with a flexible ending or two flexible endings.

A linker can comprise a hinge region or fragment thereof, for example an amino acid sequence derived from a hinge region of an antibody or immune receptor. In some embodiments, a linker comprises a hinge region from CD8α, IgG1, or IgG4 (e.g., mammalian or human).

Examples of linkers include, but are not limited to, those disclosed in SEQ ID NOs: 22-64 and 187, or repeats thereof, which can be used to link any domain or polypeptide disclosed herein to any other portion domain or polypeptide disclosed herein. A linker can comprise any one of SEQ ID NOs: 22-64 or 187, or repeats thereof, for example, 1, 2, 3, 4, 5, 6, 7, 8, 9, or 10 repeats of any one of SEQ ID NOs: 22-64 and 187. SEQ ID NOs: 22-41 and 187 provide non-limiting examples of flexible linkers or subunits thereof. SEQ ID NOs: 42-45 provide non-limiting examples of rigid linkers or subunits thereof.

TABLE 12: illustrative linkers and linker subunits.

TABLE 12

| SEQ | | |
|---|---|---|
| ID NO: | Description | Sequence |
| 22 | Linker | GSGGGGSGT |
| 23 | Linker | TGSGGGGSG |
| 24 | Linker | GGSGGGSGT |
| 25 | Linker | GSGGGGSG |
| 26 | Linker | GGSGGGSG |
| 187 | Linker | TGSGGGGSGT |
| 27 | Flexible | GG |
| | linker/linker | |
| | subunit | |
| 28 | Flexible | GGGS |
| | linker/linker | |
| | subunit | |
| 29 | Linker or | GGGGS |
| | linker | |
| | subunit | |
| 30 | Flexible | GGGGSGGGGSGGGGS |
| | linker/linker | |
| | subunit | |
| 31 | Flexible | KESGSVSSEQLAQFRSLD |
| | linker/linker | |
| | subunit | |
| 32 | Flexible | EGKSSGSGSESKST |
| | linker/linker | |
| | subunit | |
| 33 | Flexible | GSAGSAAGSGEF |
| | linker/linker | |
| | subunit | |
| 34 | (G4S)1 | GGGGSAS |
| | linker | |
| 35 | (G4S)2 | GGGGSGGGGSAS |
| | linker | |
| 36 | (G4S)3 | GGGGSGGGGSGGGGSAS |
| | linker | |
| 37 | (G4S)5 | GGGGSGGGGSGGGGSGGGGSGGGGSAS |
| | linker | |
| 38 | (G4S)6 | GGGGSGGGGSGGGGSGGGGSGGGGSGGGGSAS |
| | linker | |
| 39 | (G4S)7 | AGGGSGGGGSGGGGSGGGGSGGGGSGGGGSGGGGSAS |
| | linker | |
| 40 | (G4S)8 | AGGGSGGGGSGGGGSGGGGSGGGGSGGGGSGGGGSGGGGSA |
| | linker | S |
| 41 | (G4S)9 | AGGGSGGGGSGGGGSGGGGSGGGGSGGGGSGGGGSGGGGSG |
| | linker | GGGSAS |
| 42 | Rigid | EAAAK |
| | linker/linker | |
| | subunit | |
| 43 | Rigid | EAAAR |
| | linker/linker | |
| | subunit | |
| 44 | Rigid | PAPAP |
| | linker/linker | |
| | subunit | |
| 45 | Rigid | AEAAAKEAAAKA |
| | linker/linker | |
| | subunit | |
| 46 | IgG1 hinge | SGGGSDPAEPKSPDKTHTCPPCPKDPK |
| 47 | IgG1 hinge | AEPKSPDKTHTCPPCPKDPK |
| 48 | CD8 Hinge | TTTPAPRPPTPAPTIASQPLSLRPEACRPAAGGAVHTRGLDFAC |
| | | D |
| 49 | GPcPcPc | AGSGGSGGSGGSPVPSTPPTNSSSTPPTPSPSPVPSTPPTNSSSTP |
| | linker | PTPSPSPVPSTPPTNSSSTPPTPSPSAS |
| 50 | GPPcP linker | AGSGGSGGSGGSPVPSTPPTPSPSTPPTPSPSPVPSTPPTNSSSTPP |
| | | TPSPSPVPSTPPTPSPSTPPTPSPSAS |
| 51 | GPGcP | AGSGGSGGSGGSPVPSTPPTPSPSTPPTPSPSGGSGNSSGSGGSP |
| | linker | VPSTPPTPSPSTPPTPSPSAS |
| 52 | GPPP linker | AGSGGSGGSGGSPVPSTPPTPSPSTPPTPSPSPVPSTPPTPSPSTPP |
| | | TPSPSPVPSTPPTPSPSTPPTPSPSAS |
| 53 | GPbP linker | AGSGGSGGSGGSPVPSTPPTPSPSTPPTPSPSIQRTPKIQVYSRHP |
| | | AENGKSNFLNCYVSGFHPSDIEVDLLKNGERIEKVEHSDLSFSK |
| | | DWSFYLLYYTEFTPTEKDEYACRVNHVTLSQPKIVKWDRDPV |
| | | PSTPPTPSPSTPPTPSPSAS |
| 54 | GPbG linker | AGSGGSGGSGGSPVPSTPPTPSPSTPPTPSPSIQRTPKIQVYSRHP |
| | | AENGKSNFLNCYVSGFHPSDIEVDLLKNGERIEKVEHSDLSFSK |
| | | DWSFYLLYYTEFTPTEKDEYACRVNHVTLSQPKIVKWDRDGG |
| | | SGGSGGSGGSAS |
| 55 | PbGbG | AGPVPSTPPTPSPSTPPTPSPSIQRTPKIQVYSRHPAENGKSNFL |
| | linker | NCYVSGFHPSDIEVDLLKNGERIEKVEHSDLSFSKDWSFYLLYYT |
| | | EFTPTEKDEYACRVNHVTLSQPKIVKWDRDGGSGGSGGSGGSI |
| | | QRTPKIQVYSRHPAENGKSNFLNCYVSGFHPSDIEVDLLKNGE |
| | | RIEKVEHSDLSFSKDWSFYLLYYTEFTPTEKDEYACRVNHVTL |
| | | SQPKIVKWDRDGGSGGSGGSGAS |
| 56 | GPbGbP | AGSGGSGGSGGSPVPSTPPTPSPSTPPTPSPSIQRTPKIQVYSRH |
| | linker | PAENGKSNFLNCYVSGFHPSDIEVDLLKNGERIEKVEHSDLSFSK |
| | | DWSFYLLYYTEFTPTEKDEYACRVNHVTLSQPKIVKWDRDGG |
| | | SGGSGGSGGSIQRTPKIQVYSRHPAENGKSNFLNCYVSGFHPSD |
| | | IEVDLLKNGERIEKVEHSDLSFSKDWSFYLLYYTEFTPTEKDEY |
| | | ACRVNHVTLSQPKIVKWDRDPVPSTPPTPSPSTPPTPSPSAS |
| 57 | GPUG linker | AGSGGSGGSGGSPVPSTPPTPSPSTPPTPSPSQIFVKTLTGKTITL |
| | | EVEPSDTIENVKAKIQDKEGIPPDQQRLIFAGKQLEDGRTLSDY |
| | | NIQKESTLHLVLRLRGGGGSGGSGGSGGSAS |
| 58 | GPZP linker | AGSGGSGGSGGSPVPSTPPTPSPSTPPTPSPSDGRYSLTYIYTG |
| | | LSKHVEDVPAFQALGSLNDLQFFRYNSKDRKSQPMGLWRQVEG |
| | | MEDWKQDSQLQKAREDIFMETLKDIVEYYNDSNGSHVLQGRF |
| | | GCEIENNRSSGAFWKYYYDGKDYIEFNKEIPAWVPFDPAAQIT |
| | | KQKWEAEPVYVQRAKAYLEEECPATLRKYLKYSKNILDRQDP |
| | | PSVVVTSHQAPGEKKKLKCLAYDFYPGKIDVHWTRAGEVQEP |
| | | ELRGDVLHNGNGTYQSWVVVAVPPQDTAPYSCHVQHSSLAQ |
| | | PLVVPWEASPVPSTPPTPSPSTPPTPSAS |
| 59 | GGZGZP | AGSGGSGGSGGSGGSGGSGGSGGSDGRYSLTYIYTGLSKHVED |
| | linker | VPAFQALGSLNDLQFFRYNSKDRKSQPMGLWRQVEGMEDWK |
| | | QDSQLQKAREDIFMETLKDIVEYYNDSNGSHVLQGRFGCEIEN |
| | | NRSSGAFWKYYYDGKDYIEFNKEIPAWVPFDPAAQITKQKWE |
| | | AEPVYVQRAKAYLEEECPATLRKYLKYSKNILDRQDPPSVVVT |
| | | SHQAPGEKKKLKCLAYDFYPGKIDVHWTRAGEVQEPELRGDV |
| | | LHNGNGTYQSWVVVAVPPQDTAPYSCHVQHSSLAQPLVVPW |
| | | EASGGSGGSGGSGGSDGRYSLTYIYTGLSKHVEDVPAFQALGS |
| | | LNDLQFFRYNSKDRKSQPMGLWRQVEGMEDWKQDSQLQKA |
| | | REDIFMETLKDIVEYYNDSNGSHVLQGRFGCEIENNRSSGAFW |
| | | KYYYDGKDYIEFNKEIPAWVPFDPAAQITKQKWEAEPVYVQR |
| | | AKAYLEEECPATLRKYLKYSKNILDRQDPPSVVVTSHQAPGEK |
| | | KKLKCLAYDFYPGKIDVHWTRAGEVQEPELRGDVLHNGNGT |
| | | YQSWVVVAVPPQDTAPYSCHVQHSSLAQPLVVPWEASPVPST |
| | | PPTPSPSTPPTPSPSAS |
| 60 | GcGcP linker | AGSGNSSGSGGSGGSGNSSGSGGSPVPSTPPTPSPSTPPTPS |
| | | PSAS |
| 61 | cTPR3 linker | KLSGGGGSGGGGSGGGGSAEAWYNLGNAYYKQGDYQKAIEY |
| | | YQKALELDPNNAEAWYNLGNAYYKQGDYQKAIEYYQKALEL |
| | | DPNNAEAWYNLGNAYYKQGDYQKAIEDYQKALELDPNNLQR |
| | | SAGGGGSGGGGSGGGGAS |
| 62 | cTPR6 linker | KLSGGGGSGGGGSGGGGSAEAWYNLGNAYYKQGDYQKAIEY |
| | | YQKALELDPNNAEAWYNLGNAYYKQGDYQKAIEYYQKALEL |
| | | DPNNAEAWYNLGNAYYKQGDYQKAIEDYQKALELDPNNLQA |
| | | EAWKNLGNAYYKQGDYQKAIEYYQKALELDPNNASAWYNL |
| | | GNAYYKQGDYQKAIEYYQKALELDPNNAKAWYRRGNAYYK |
| | | QGDYQKAIEDYQKALELDPNNRSRSAGGGGSGGGGSGGGGAS |
| 63 | cTPR9 linker | KLSGGGGSGGGGSGGGGSAEAWYNLGNAYYKQGDYQKAIEY |
| | | YQKALELDPNNAEAWYNLGNAYYKQGDYQKAIEYYQKALEL |
| | | DPNNAEAWYNLGNAYYKQGDYQKAIEDYQKALELDPNNLQA |
| | | EAWKNLGNAYYKQGDYQKAIEYYQKALELDPNNASAWYNL |
| | | GNAYYKQGDYQKAIEYYQKALELDPNNAKAWYRRGNAYYK |
| | | QGDYQKAIEDYQKALELDPNNRSAEAWYNLGNAYYKQGDYQ |
| | | KAIEYYQKALELDPNNAEAWYNLGNAYYKQGDYQKAIEYYQ |
| | | KALELDPNNAEAWYNLGNAYYKQGDYQKAIEDYQKALELDP |
| | | NNLQRSAGGGGSGGGGSGGGGAS |
| 64 | cTPR12 | KLSGGGGSGGGGSGGGGSAEAWYNLGNAYYKQGDYQKAIEY |
| | linker | YQKALELDPNNAEAWYNLGNAYYKQGDYQKAIEYYQKALEL |
| | | DPNNAEAWYNLGNAYYKQGDYQKAIEDYQKALELDPNNLQA |
| | | EAWKNLGNAYYKQGDYQKAIEYYQKALELDPNNASAWYNL |
| | | GNAYYKQGDYQKAIEYYQKALELDPNNAKAWYRRGNAYYK |
| | | QGDYQKAIEDYQKALELDPNNRSAEAWYNLGNAYYKQGDYQ |
| | | KAIEYYQKALELDPNNAEAWYNLGNAYYKQGDYQKAIEYYQ |
| | | KALELDPNNAEAWYNLGNAYYKQGDYQKAIEDYQKALELDP |
| | | NNLQAEAWKNLGNAYYKQGDYQKAIEYYQKALELDPNNASA |
| | | WYNLGNAYYKQGDYQKAIEYYQKALELDPNNAKAWYRRGN |
| | | AYYKQGDYQKAIEDYQKALELDPNNRSAGGGGSGGGGSGGG |
| | | GAS |
| 192 | Ubiquitin C- | MQIFVKTLTGKTITLEVEPSDTIENVKAKIQDKEGIPPDQQRL |
| | terminal tag | IFAGKQLEDGRTLSDYNIQKESTLHLVLRLRGG |
| 193 | Ubiquitin | MQIFVRTLTDRTITLEVEPSDTIENVRARIQDREGIPPDQQRL |
| | acceptor | IFAGRQLEDGRTLSDYNIQKESTLHLVLRPRVVD |

In some embodiments, a linker comprises a ubiquitin monomer. In some embodiments, a linker comprises two ubiquitin monomers. In some embodiments, a linker comprises two or more ubiquitin monomers.

A fusion polypeptide, engineered immunocytokine, immunoglobulin constant domain, or component thereof can comprise a ubiquitin linker that comprises one or more ubiquitin monomers. A ubiquitin linker can act as a rigid linker that maintains the spacing and orientation between components bound to the ubiquitin linker.

In some embodiments, a ubiquitin linker is used to join two components of a fusion polypeptide or engineered immunocytokine in a linear manner.

In some embodiments, a ubiquitin linker is used to join two components of a fusion polypeptide or engineered immunocytokine in a branched manner. For example, in some embodiments a ubiquitin linker joins a stabilizing domain or immunoglobulin constant domain to two cytokines disclosed herein, wherein each of the cytokines are joined to the ubiquitin in a branched fashion. In this way, the two cytokines (e.g., IL4 and IL10, IL4 and IL13, or IL10 and IL13) can be joined to a single immunoglobulin constant region (e.g., Fc domain, or single chain Fc domain) in a branched fashion. In some embodiments, use of a branched linker, such as a branch ubiquitin linker, can allow for a molecule disclosed herein to be produced without requiring heterodimeric association of immunoglobulin polypeptide chains, such as heavy chains.

A fusion polypeptide, engineered immunocytokine, immunoglobulin constant domain, or component thereof can be joined to the N-terminus or the C-terminus of ubiquitin, optionally via another linker.

A C-terminal site of a ubiquitin monomer can be conjugated with a lysine of a second ubiquitin monomer. A ubiquitin linker can be formed by covalently bonding a donor ubiquitin, e.g., in which one or more lysines of the ubiquitin are substituted with other amino acids such as arginine or alanine, and an acceptor ubiquitin, e.g., in which the lysine at residue 6, 11, 27, 29, 33, 48, and/or 63 of ubiquitin is substituted with another amino acid such as arginine or alanine. In some embodiments, leucine at position 73 of ubiquitin is substituted with another amino acid, such as proline. In some embodiments, a donor and/or acceptor ubiquitin comprises substitutions of one or more lysine residues. In some embodiments, a donor and/or acceptor ubiquitin comprises substitutions of all lysine residues except for K48, e.g., lysine to alanine or lysine to arginine. In some embodiments, a donor and/or acceptor ubiquitin comprises substitutions of all lysine residues except for K63, e.g., lysine to alanine or lysine to arginine. In some embodiments, two or more ubiquitins are repeatedly linked in a head-to-tail form or in a branched form (branched type or iso-peptide branch type form). The glycines at positions 75 and/or 76 of the ubiquitin can be substituted with another amino acid, such as valine.

A fusion polypeptide, engineered immunocytokine, or component thereof can be reacted (e.g., in vitro) with a ubiquitination-related enzyme. In some embodiments, two components of a fusion polypeptide or engineered immunocytokine are joined by via two or more ubiquitin monomers, e.g., as a linker. The ubiquitin monomers can be covalently bound via treating with enzymes E1 (activation enzyme), E2 (conjugation enzyme) and/or E3 (ligase), for example, E1, E2 and E3, or E1 and E2, or treating with E2-25K ubiquitin conjugating enzyme or Ucb13-MMS2, a ubiquitin conjugating enzyme complex. An illustrative buffer that can be used for the ubiquitin conjugating reaction is 25 mM HEPES, pH 7.5, 50 mM NaCl, 4 mM MgCl2, with addition of 0.5 μM E1, 5 μM E2, 1 μM E3, 4 mM ATP to initiate the reaction.

An illustrative acceptor ubiquitin sequence is provided in SEQ ID NO: 193. An illustrative ubiquitin C-terminal tag is provided in SEQ ID NO: 192.

A linker of the disclosure can include a chemical linker. For example, two compounds (e.g., polypeptides) of the disclosure can be connected together by a chemical linker. Each chemical linker of the disclosure can be alkylene, alkenylene, alkynylene, heteroalkylene, cycloalkylene, heterocycloalkylene, arylene, or heteroarylene, any of which is optionally substituted. In some embodiments, a chemical linker of the disclosure can be an ester, ether, amide, thioether, or polyethyleneglycol (PEG). Non-limiting examples of such linkers include diesters of dicarboxylic acids, such as oxalyl diester, malonyl diester, succinyl diester, glutaryl diester, adipyl diester, pimetyl diester, fumaryl diester, maleyl diester, phthalyl diester, isophthalyl diester, and terephthalyl diester. Non-limiting examples of such linkers include diamides of diamino linkers, such as ethylene diamine, 1,2-di(methylamino)ethane, 1,3-diaminopropane, 1,3-di(methylamino)propane, 1,4-di(methylamino)butane, 1,5-di(methylamino)pentane, 1,6-di(methylamino)hexane, and pipyrizine.

A linker can be a cleavable linker. A linker can be a non-cleavable linker.

A nucleic acid encoding two or more polypeptides disclosed herein can be designed to encode the two or more polypeptides linked by one or more 2A linkers, which can be processed into separate polypeptides co-translationally or after translation. Inclusion of a 2A linker can increase the likelihood that an appropriate ratio of components are produced (e.g., a 1:1, 1:2, 1:3, 1:4, or 1:5 ratio of two components). In some cases, inclusion of a 2A linker can increase the likelihood that equal or close to equal levels of two components of an engineered immunocytokine are produced, e.g., a first heavy polypeptide chain and a second polypeptide chain, each containing an immunoglobulin constant domain joined to a cytokine. In some cases, use of a 2A linker can allow for fewer components in a system for transgene expression and/or genome modification, e.g., inclusion of multiple components in one vector rather than separate vectors.

A nucleic acid encoding a polypeptide of the disclosure can encode a signal peptide. In some cases, a polypeptide of the disclosure comprises a signal peptide. A signal peptide can be cleaved off during processing of the protein, thus in some cases a mature polypeptide disclosed herein does not contain a signal peptide.

A signal peptide at the N-terminus of a protein can be involved in transport of the protein to or through a membrane, transport to different a membranous cellular compartment, or secretion of the protein from the cell. A nucleic acid encoding a polypeptide of the disclosure can encode a signal peptide to facilitate secretion of the polypeptide. A signal peptide can be selected for its ability to facilitate ER processing and secretion of the polypeptide. Any suitable signal peptide can be used. A signal peptide can be about 10 to about 40 amino acids in length. In some cases, a signal peptide is at least about 10, 15, 16, 20, 21, 22, 25, or 30 amino acids in length, or more. In some cases, a signal peptide is at most about 15, 16, 20, 21, 22, 25, or 30 amino acids in length, or less. In some cases, a signal peptide is about 16-30 amino acids in length.

A polypeptide (e.g., cytokine, engineered IL10 polypeptide, fusion polypeptide, or immunocytokine) disclosed herein can comprise an added amino acid sequence at the N- and/or C-terminus, e.g., an affinity tag to facilitate purification. For example, a poly-histidine-tag, GST-tag, FLAG-tag, CBP tag, HA tag, or Myc tag can be present at the C- or N-terminus to facilitate purification. In some embodiments, an affinity tag is removed from a protein of the disclosure, e.g., after purification. In some embodiments, a polypeptide of the disclosure does not contain an affinity tag, (e.g., the polypeptide can be purified by other methods).

### H. Pharmacokinetics

Fusion polypeptides and engineered immunocytokines disclosed herein can have advantageous pharmacokinetic properties, for example, increased half-life and/or prolonged therapeutic efficacy.

In some embodiments, a fusion polypeptide or engineered immunocytokine exhibits a plasma half-life of at least about 3 hours, at least about 6 hours, at least about 12 hours, at least about 18 hours, at least about 24 hours, at least about 36 hours, at least about 48 hours, at least about 72 hours, at least about 5 days, at least about 7 days, at least about 10 days, at least about 14 days, at least about 21 days, at least about 28 days, at least about 35 days, at least about 42 days, at least about 49 days, at least about 60 days, at least about 75 days, at least about 100 days, at least about 125 days, at least about 150 days, or at least about 180 days, for example, after intravenous administration. In some embodiments, the intravenous administration is to a human. In some embodiments, the intravenous administration is to a non-human mammal.

In some embodiments, a fusion polypeptide or engineered immunocytokine exhibits a half-life in a target tissue of at least about 3 hours, at least about 6 hours, at least about 12 hours, at least about 18 hours, at least about 24 hours, at least about 36 hours, at least about 48 hours, at least about 72 hours, at least about 5 days, at least about 7 days, at least about 10 days, at least about 14 days, at least about 21 days, at least about 28 days, at least about 35 days, at least about 42 days, at least about 49 days, at least about 60 days, at least about 75 days, at least about 100 days, at least about 125 days, at least about 150 days, or at least about 180 days after administration, for example, parenteral, local, or systemic administration. In some embodiments, the administration is to a human. In some embodiments, the administration is to a non-human mammal.

A half-life can be determined, for example, by a pharmacokinetic assay in a suitable animal, e.g., rodent, primate, or human. In some embodiments, the assay is conducted with a fusion polypeptide or engineered immunocytokine that comprises a non-human (e.g., murine) cytokine and/or immunoglobulin constant domain, e.g., to reduce immunogenicity.

In some embodiments, a fusion polypeptide or engineered immunocytokine exhibits a therapeutic activity, for example, a reduction of pain or hyperalgesia that is significant and/or to below a certain threshold, a reduction of inflammation that is significant and/or to below a certain threshold, or a reduction of neuropathy or neurodegeneration that is significant and/or to below a certain threshold, for at least about 3 hours, at least about 6 hours, at least about 12 hours, at least about 18 hours, at least about 24 hours, at least about 36 hours, at least about 48 hours, at least about 72 hours, at least about 5 days, at least about 7 days, at least about 10 days, at least about 14 days, at least about 21 days, at least about 28 days, at least about 35 days, at least about 42 days, at least about 49 days, at least about 60 days, at least about 75 days, at least about 100 days, at least about 125 days, at least about 150 days, or at least about 180 days after administration, for example, parenteral, local, or systemic administration.

### Nucleic Acids and Vectors

In some embodiments the disclosure provides a nucleic acid that encodes a cytokine (e.g., engineered IL10 polypeptide), fusion polypeptide, or engineered immunocytokine disclosed herein. The nucleic acid can be prepared by standard molecular biology techniques. The nucleic acid can be prepared by molecular cloning. The nucleic acid can be synthesized de novo. The nucleic acid can comprise a nucleotide sequence encoding the cytokine (e.g., engineered IL10 polypeptide), fusion polypeptide, or engineered immunocytokine, operably linked to transcription regulatory sequences such as a promoter, and optionally a 3′ untranslated region. A constitutive, inducible, or tissue-specific promoter can be used.

The nucleic acid can be a DNA. The nucleic acid can be an RNA. The nucleic acid can comprise a modified base, for example, to enhance stability of the nucleic acid upon administration to a subject. A nucleic acid provided can include a recombinant, artificial, or synthetic nucleic acid. The nucleic acid can be single stranded. The nucleic acid can be double stranded. The nucleic acid can be recombinant and/or isolated.

The nucleic acid can be inserted into or part of a vector, such as an expression vector, such that the genes are operatively linked to transcriptional and/or translational control sequences. The vector can comprise a selectable marker for selection of a vector-carrying host cell. The vector can lack a selectable marker. The vector can comprise an origin of replication or can lack an origin of replication. The vector can be a plasmid, for example, a nanoplasmid. The vector can be a minicircle. The vector can be a liner nucleic acid phagemid, cosmid, RNA vector, viral vector or the like. Non-limiting examples of viral vectors include a retrovirus (e.g., lentivirus), an adenovirus, and an adeno-associated virus.

In some embodiments, a nucleic acid encoding a cytokine, fusion polypeptide, or engineered immunocytokine is packaged in a lipid-based delivery vector, such as a liposome or lipid nanoparticle.

Regulatory sequences for mammalian host cell expression can include viral elements that direct high levels of protein expression in mammalian cells, such as promoters and/or enhancers derived from cytomegalovirus (CMV), Simian Virus 40 (SV40), and adenovirus, (e.g., the adenovirus major late promoter (AdMLP)). Nonviral regulatory sequences can be used, such as the ubiquitin promoter, or a cell type-specific or tissue-specific promoter. An inducible and/or repressible promoter can be used.

In some embodiments the disclosure provides a host cell comprising a nucleic acid that encodes a cytokine (e.g., engineered IL10 polypeptide), fusion polypeptide, or engineered immunocytokine. The host cell can contain the nucleic acid or vector as an extra-chromosomally (episomal) replicating molecule, or integrated in the genome of the host cell. In some embodiments, a vector or host cell includes an enzyme or a gene encoding an enzyme for post-translational modification of the cytokine, fusion polypeptide, or engineered immunocytokine, such as glycosylation.

A cytokine (e.g., engineered IL10 polypeptide), fusion polypeptide, or engineered immunocytokine disclosed herein can be prepared using standard techniques. In some embodiments, the cytokine, fusion polypeptide, or engineered immunocytokine is produced by a continuous cell line in culture, e.g., a mammalian cell line such as a HEK cell line, CHO cell line, COS cell line, or other suitable cell line. When recombinant expression vectors comprising nucleic acid sequences encoding a cytokine, fusion polypeptide, or engineered immunocytokine are introduced into host cells, proteins can be produced by culturing the host cells for a period of time sufficient for expression of the proteins by the host cells and secretion into the culture medium in which the host cells are grown. The proteins can be recovered from the culture medium using protein purification methods. For optimal expression in a host cell, the DNA sequence encoding a protein of the disclosure can be codon-optimized by adapting the codon usage to a profile suitable or preferable for the host cell.

In an aspect, the disclosure provides a method for producing a cytokine, fusion polypeptide, or engineered immunocytokine, the method comprising culturing a host cell of the under conditions permitting production of the protein, and optionally recovering the protein.

In some embodiments, a cytokine, fusion polypeptide, or engineered immunocytokine is expressed in prokaryotic cells, such as microorganisms, e.g. E. coli, or in an algal expression systems insect cell expression systems or cell-free protein synthesis systems. In some embodiments, the cytokine, fusion polypeptide, or engineered immunocytokine is produced in a bacterial culture. In some embodiments, the cytokine, fusion polypeptide, or engineered immunocytokine is produced in a cell-free expression system.

In some embodiments, the cytokine, fusion polypeptide, or engineered immunocytokine is be synthesized de novo by chemical synthesis (using e.g. a peptide synthesizer).

A cytokine, fusion polypeptide, or engineered immunocytokine can be recovered or purified via techniques that include, without limitation, chromatographic methods (including, without limitation, size exclusion chromatography, hydrophobic interaction chromatography, ion exchange chromatography, affinity chromatography, immunoaffinity chromatography, metal binding, and the like), immunoprecipitation, HPLC, ultracentrifugation, precipitation and differential solubilisation, and extraction. Recovery or purification of the protein may be facilitated by adding, for example, a poly-His-tag to the fusion protein.

### Pharmaceutical Compositions

Compositions disclosed herein can comprise a polypeptide and a pharmaceutically-acceptable excipient, vehicle, carrier, or diluent. For example, in some embodiments the disclosure provides a pharmaceutical composition comprising a cytokine (e.g., interleukin, IL10 polypeptide, engineered IL10 polypeptide, IL4, IL13, or a variant, derivative, or functional fragment thereof) and a pharmaceutically-acceptable excipient, vehicle, carrier, or diluent. In some embodiments, the disclosure provides a pharmaceutical composition that comprises a fusion polypeptide and a pharmaceutically-acceptable excipient, vehicle, carrier, or diluent. The fusion polypeptide can be, for example, a fusion polypeptide comprising an engineered IL10 polypeptide (e.g., joined to a second cytokine, such as IL4 or IL13, optionally via a linker). The fusion polypeptide can be an engineered immunocytokine (e.g., a heterodimeric immunocytokine comprising an Fc fragment and cytokines, such as an IL4 polypeptide joined to one chain and an IL10 polypeptide joined to the second chain, an IL4 polypeptide joined to one chain and an IL13 polypeptide joined to the second chain, or an IL13 polypeptide joined to one chain and an IL10 polypeptide joined to the second chain). Compositions disclosed herein can comprise a nucleic acid or a vector and a pharmaceutically-acceptable excipient, vehicle, carrier, or diluent.

A pharmaceutical composition disclosed herein can comprise a saline solution. A pharmaceutical composition disclosed herein can comprise a buffered saline solution, for example, PBS, dPBS, HBSS, or the like. A pharmaceutical composition disclosed herein can comprise Ringer's solution, dextrose solution, or Hank's solution.

A pharmaceutical composition disclosed herein can comprise a buffer, for example, a citrate buffer (e.g., sodium citrate) or a phosphate buffer (e.g., sodium phosphate buffer). A pharmaceutical composition disclosed herein can comprise a pH-stabilizing agent.

A pharmaceutical composition disclosed herein can comprise an organic co-solvent, e.g., polysorbate 20, polysorbate 80, propylene glycol, or polyethylene glycol (PEG).

A pharmaceutical composition disclosed herein can comprise a stabilizing agent, e.g., sucrose, sorbitol, glycerol, trehalose, or mannitol.

A pharmaceutical composition disclosed herein can comprise a tonicity agent, e.g., a salt, such as NaCl or KCl.

A pharmaceutical composition disclosed herein can comprise a preservative. A pharmaceutical composition disclosed herein can comprise an antimicrobial gent. A pharmaceutical composition disclosed herein can comprise an antifungal agent.

In some embodiments, a polypeptide disclosed herein is in an aqueous buffer. In some embodiments, a polypeptide disclosed herein is in a powdered (e.g., lyophilized) form.

Non-limiting examples of pharmaceutically-acceptable excipients, vehicles, carriers, and diluents can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), and updated versions thereof, each of which is incorporated by reference in its entirety.

A cytokine, engineered IL10 polypeptide, fusion polypeptide, or engineered immunocytokine can be present as, purified into, and/or used as monomer. A cytokine, engineered IL10 polypeptide, fusion polypeptide, or engineered immunocytokine can be present as, purified into, and/or used as dimer.

In some embodiments, the disclosure provides a composition comprising an engineered IL10 polypeptide, or a pharmaceutical composition comprising the engineered IL10 polypeptide and a pharmaceutically-acceptable excipient, vehicle, carrier, or diluent. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99%, or at least 99.5% of the engineered IL10 polypeptide is in a monomeric form. In some embodiments, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 25%, at most 30%, at most 35%, at most 40%, at most 45%, at most 50%, at most 55%, at most 60%, at most 65%, at most 70%, at most 75%, or at most 80% of the engineered IL10 polypeptide is in a dimeric or multimeric form. In some embodiments, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 25%, at most 30%, at most 35%, at most 40%, at most 45%, at most 50%, at most 55%, at most 60%, at most 65%, at most 70%, at most 75%, or at most 80% of the engineered IL10 polypeptide is in an IL10 homodimeric form. The engineered IL10 polypeptide can be present in a fusion polypeptide, e.g., joined to a second cytokine, IL4, or IL13 polypeptide, optionally via a linker. The engineered IL10 polypeptide can be present in an engineered immunocytokine (e.g., a heterodimeric immunocytokine comprising an Fc fragment with an IL4 polypeptide joined to one chain and the engineered IL10 polypeptide joined to the second chain, or an IL13 polypeptide joined to one chain and the engineered IL10 polypeptide joined to the second chain).

In some embodiments, the disclosure provides a composition comprising a fusion polypeptide (e.g., that comprises an engineered IL10 polypeptide), or a pharmaceutical composition comprising the fusion polypeptide (e.g., that comprises an engineered IL10 polypeptide) and a pharmaceutically-acceptable excipient, vehicle, carrier, or diluent. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99%, or at least 99.5% of the fusion polypeptide is in a monomeric form. In some embodiments, at most 1%, at most 2%, at most 3%, at most 4%, at most 5%, at most 6%, at most 7%, at most 8%, at most 9%, at most 10%, at most 110, at most 12%, at most 13%, at most 14%, at most 15%, at most 16%, at most 17%, at most 18%, at most 19%, at most 20%, at most 25%, at most 30%, at most 35%, at most 40%, at most 45%, at most 50%, at most 55%, at most 60%, at most 65%, at most 70%, at most 75%, or at most 80% of the fusion polypeptide is in a dimeric or multimeric form. In some embodiments, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 25%, at most 30%, at most 35%, at most 40%, at most 45%, at most 50%, at most 55%, at most 60%, at most 65%, at most 70%, at most 75%, or at most 80% of the fusion polypeptide is in an IL10 homodimeric form. The fusion polypeptide can comprise an engineered IL10 polypeptide e.g., joined to an IL4 or IL13 polypeptide, optionally via a linker.

In some embodiments, the disclosure provides a composition comprising an engineered immunocytokine (e.g., that comprises an engineered IL10 polypeptide), or a pharmaceutical composition comprising the engineered immunocytokine (e.g., that comprises an engineered IL10 polypeptide) and a pharmaceutically-acceptable excipient, vehicle, carrier, or diluent.

In some embodiments, at most 1%, at most 2%, at most 3%, at most 4%, at most 5%, at most 6%, at most 7%, at most 8%, at most 9%, at most 10%, at most 110, at most 12%, at most 13%, at most 14%, at most 15%, at most 16%, at most 17%, at most 18%, at most 19%, at most 20%, at most 25%, at most 30%, at most 35%, at most 40%, at most 45%, at most 50%, at most 55%, at most 60%, at most 65%, at most 70%, at most 75%, or at most 80% of the engineered immunocytokine is in an IL10 homodimeric form. For example, the engineered immunocytokine can be a heterodimeric immunocytokine comprising an Fc fragment with two chains that form a dimer (e.g., one with an engineered IL10 polypeptide appended and the other with a second cytokine appended), and dimerization of the engineered immunocytokine with a second copy of the engineered immunocytokine or with IL10 can be substantially reduced, e.g., to no more than a recited percentage. In some embodiments, the engineered immunocytokine comprises an engineered IL10 polypeptide and at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99%, or at least 99.5% of the engineered immunocytokine is not bound to a second IL10 polypeptide. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99%, or at least 99.5% of the engineered immunocytokine is in a monomeric form. In some embodiments, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 25%, at most 30%, at most 35%, at most 40%, at most 45%, at most 50%, at most 55%, at most 60%, at most 65%, at most 70%, at most 75%, or at most 80% of the engineered immunocytokine is in a dimeric or multimeric form. The engineered immunocytokine can be a heterodimeric immunocytokine comprising an Fc fragment with an IL4 polypeptide joined to one chain and an engineered IL10 polypeptide joined to the second chain. The engineered immunocytokine can be a heterodimeric immunocytokine comprising an Fc fragment with an IL13 polypeptide joined to one chain and an engineered IL10 polypeptide joined to the second chain.

### METHODS

The disclosure encompasses methods of treating a subject and compositions for use in a method of treating a subject. For example, a cytokine (e.g., engineered IL10 polypeptide), fusion polypeptide, or engineered immunocytokine disclosed herein can be useful for treating a condition in a subject in need thereof.

In some embodiments, provided is a method of treating a condition in a subject in need thereof, the method comprising administering to the subject an effective amount of a cytokine (e.g., engineered IL10 polypeptide), fusion polypeptide, or engineered immunocytokine disclosed herein. In some embodiments, provided is a method of treating a condition in a subject in need thereof, the method comprising administering to the subject an effective amount of a pharmaceutical composition comprising a cytokine (e.g., engineered IL10 polypeptide), fusion polypeptide, or engineered immunocytokine disclosed herein. In some embodiments, provided is a method of treating a condition in a subject in need thereof, the method comprising administering to the subject an effective amount of a nucleic acid encoding a cytokine (e.g., engineered IL10 polypeptide), fusion polypeptide, or engineered immunocytokine disclosed herein, or a vector comprising the nucleic acid. The effective amount can be a therapeutically-effective amount.

The condition can be, be characterized by, or comprise pain. The condition can be, be characterized by, or comprise chronic pain. The condition can be, be characterized by, or comprise neuropathic pain. The condition can be, be characterized by, or comprise inflammatory pain. The condition can be, be characterized by, or comprise inflammation. The condition can be, be characterized by, or comprise chronic inflammation. The condition can be, be characterized by, or comprise osteoarthritis. The condition can be, be characterized by, or comprise neuroinflammation. The condition can be, be characterized by, or comprise neuropathy. The condition can be, be characterized by, or comprise chemotherapy-induce neuropathy. The condition can be, be characterized by, or comprise neurodegeneration.

The condition can be a disorder associated with mitochondrial dysfunction, such as a neurodegenerative disorder, diabetes, a musculoskeletal disorder, or a cardiovascular disorder.

The subject can be a mammalian subject. The subject can be a human subject. In some embodiments, the subject is a murine, rodent, canine, feline, equine, porcine, primate, or bovine subject. In some embodiments, the subject is a non-human and/or non-rodent mammalian subject.

The cytokine (e.g., engineered IL10 polypeptide), fusion polypeptide, or engineered immunocytokine can be administered to the subject via local administration. The cytokine (e.g., engineered IL10 polypeptide), fusion polypeptide, or engineered immunocytokine can be administered to the subject via systemic administration. The cytokine (e.g., engineered IL10 polypeptide), fusion polypeptide, or engineered immunocytokine can be administered to the subject via parenteral administration.

In some embodiments, the cytokine (e.g., engineered IL10 polypeptide), fusion polypeptide, or engineered immunocytokine is administered to the subject via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, intracerebral, intraocular, intralesional, intracerebroventricular, intracisternal, or intraparenchymal administration, e.g., injection or infusion. In some embodiments, the cytokine (e.g., engineered IL10 polypeptide), fusion polypeptide, or engineered immunocytokine is administered to the subject via intravenous administration. In some embodiments, the cytokine (e.g., engineered IL10 polypeptide), fusion polypeptide, or engineered immunocytokine is administered to the subject via subcutaneous administration.

### EXAMPLES

The following examples are included for illustrative purposes only and are not intended to limit the scope of the disclosure.

### I. Example 1: Production and Purification of Fusion Polypeptides Comprising Engineered IL10

This example describes production and purification of fusion polypeptides disclosed herein that comprise an IL4 polypeptide and an engineered IL10 polypeptide (e.g., recombinant fusion polypeptides of SEQ ID NO: 66 and SEQ ID NO: 68). The fusion polypeptide of SEQ ID NO: 66 comprises IL4 joined to an engineered IL10 with rearranged helices to promote retention of the IL10 in a monomeric state (A helix joined to F helix, wherein the C-terminus of the D helix is not joined to the N-terminus of the E helix. The fusion polypeptide of SEQ ID NO: 68 comprises IL4 joined to an engineered IL10 that comprises an insertion between the D and E helices to promoter retention of the IL10 in a monomeric state.

HEK293E cells were transiently transfected according to standard procedures with a vector containing the relevant transgene. The cytokine fusion polypeptide insert was cloned in a pUPE expression vector containing a signal peptide. HEK293E cells were then transfected with the expression vector containing a cytokine fusion polypeptide. Six days post transfection, conditioned medium containing recombinant protein was harvested by low-speed centrifugation (10 minutes, 1000×g) followed by high-speed centrifugation (10 minutes, 4000×g). The recombinant protein was bound to 0.5 mL Nickel Excel sepharose. Nickel Excel Sepharose bound protein was harvested by centrifugation and transferred into a gravity flow column. Non-specifically bound proteins were removed by washing the column with a buffer containing 25 mM Tris 500 mM NaCl (pH 8.2) with 0 mM and 10 mM imidazol. The protein was eluted with a buffer containing 25 mM Tris 500 mM NaCl (pH 8.2) and 500 mM imidazol. Fractions of 2.5 mL were collected. Recombinant protein-containing fractions were pooled. The IMAC pool was concentrated to 2-4 mL using an Amicon 10 kDa spin filter. Aggregates were removed by centrifugation (10 minutes 18000×g, 4° C.). The proteins were purified further by gel filtration using a Superdex200 16/600 column equilibrated in PBS. Protein containing fractions were analyzed by LabChip capillary electrophoresis and recombinant protein containing fractions were pooled. The pool was sterilized by filtration over a 0.22 μm syringe filter and the product stored at −80° C.

Protein concentrations of purified preparations were determined using A280 absorbance applying a protein-specific extinction coefficient.

### J. Example 2: Production and Purification of Engineered Immunocytokines

This example describes production and purification of engineered immunocytokines. The engineered immunocytokines produced were Fc fusions with an IL4 polypeptide joined to the N-terminus of a modified IgG1 Fc chain via a flexible linker (e.g., SEQ ID NO: 108) and an IL10 or IL13 polypeptide joined to the N-terminus of a second modified IgG1 Fc chain via a flexible linker (e.g., SEQ ID NO: 110 or 111 for IL10, SEQ ID NO: 109 for IL13). The Fc portion of the polypeptide chains comprises knob-in-hole heterodimerization domain to facilitate pairing of the chains to form heterodimeric immunocytokines.

HEK293E cells were transiently transfected according to standard procedures. Cells were co-transfected using two vectors, each containing a transgene for a cytokine fused Fc-construct. Pairs of expression constructs were co-transfected to obtain heterodimeric Fc-fusion constructs (e.g., IL4 and IL10, or IL4 and IL13). Where needed, the transfection ratio of the two constructs was optimized to maximize heterodimerization. Six days post transfection, conditioned medium containing the recombinant protein, was harvested, sterile filtered, and stored at 4° C.

The Fc-fusion engineered immunocytokine construct was purified with a HiTrap Fibro PrismA column equilibrated in 20 mM Tris 150 mM NaCl (pH 7.8) on an Akta pure system, or the Fc-fusion engineered immunocytokine construct was bound in batch to 1 mL MabSelect PrismA (4-5 hour, 20° C.), and MabSelect PrismA containing the immunocytokine construct was collected by centrifugation and transferred to 1 gravity-flow column. Non-specifically bound proteins were removed by washing the column with PBS, PBS containing 1 M NaCl, and PBS. The bound Fc-fusion construct was eluted using 20 mM citrate and 150 mM NaCl (pH 3.0) and 5 mL fractions were collected in 15 mL tubes with direct mixing-in 1 mL of 1 M K2HPO4/KH2PO4 (pH 8.0) buffer for neutralization to pH 7. The HiTrap Fibro PrismA column was regenerated with 0.1 M NaOH.

The PrismA purification pools were concentrated using an Amicon 30 kDa spin filter and aggregates in the concentrated pools were removed by centrifugation. The recombinant antibody was further purified by gel filtration using a superdex200 Increase 16/40 column equilibrated in PBS. Protein-containing fractions were analyzed by LabChip capillary electrophoresis and Fc-fusion containing fractions were pooled. The concentrated pool was sterilized by filtration using a 0.22 μm syringe filter and stored at −80° C.

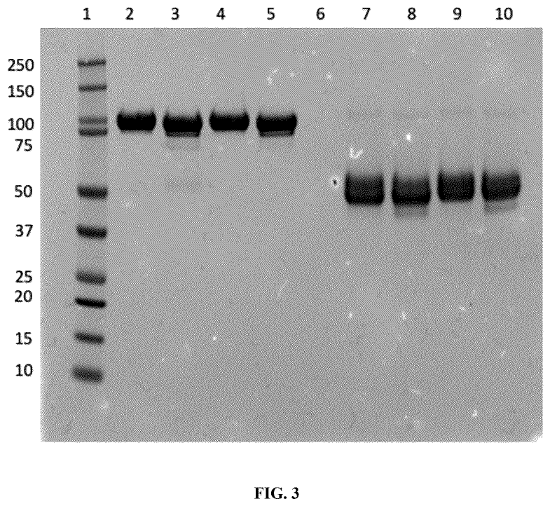

The heterodimeric immunocytokines were run on a NuPage protein gel with a 4-12% gradient in polyacrylamide concentration under non-reduced and reduced conditions to confirm heterodimeric confirmation (FIG. 3). After gel filtration, the protein-containing fractions were split in two pools. In the P1 pools (lanes 2, 4, 7, 9), the heterodimeric protein runs as a single band under non-reducing conditions, whereas under reducing conditions two bands, partially overlapping because of the close molecular size of both monomers, can be observed. Lanes 2-5 show results for non-reducing conditions, lanes 7-10 show results for reducing conditions. Lanes 2, 3, 7, and 8 are an engineered IL4-IL10 immunocytokine with SEQ ID NO: 108 paired with SEQ ID NO: 111. Lanes 4, 5, 9, and 10 are an engineered IL4-IL10 immunocytokine with SEQ ID NO: 108 paired with SEQ ID NO: 110.

P1 pools were the purest pools considered to contain a large percentage of heterodimeric protein and were used for further experimentation. Based on individual expression of the monomers, the lower band appearing in the reduced preparations of P1 pools indicate a higher percentage of IL10M containing monomer.

Labchip capillary electrophoresis was used to make an estimate on protein purity.

### K. Example 3: Receptor Signaling in Response to Fusion Polypeptides and Engineered Immunocytokines

Bioactivity at single cytokine receptors was determined using HEK-Blue™ reporter cell lines (InvivoGen), based on STAT-dependent secreted embryonic alkaline phosphatase (SEAP) expression. The HEK Blue™ IL10 reporter was used to measure IL10-induced and STAT-3 mediated induction of SEAP expression, whereas the HEK Blue™ IL4/IL13 reporter cell line was used to determine IL4 or IL13 bioactivity via STAT6-induced SEAP expression. Cells were seeded in a 96-well cell culture plate at 50,000 cells/well in a volume of 180 μL and placed in an incubator to settle overnight. The next day, 20 μL of compound solution was added to each well and incubated for 24 h in an incubator at 37° C. in 5% CO2. The next day, 20 μL of cell culture supernatant was transferred to a multi-well plate containing 180 μL of QUANTI Blue™ solution (InvivoGen) per well and incubated for 60 mins at 37° C. in the dark before the optical density (OD) was measured at 650 nM using a microplate reader. OD-values were blank-corrected before concentration-response data were fitted using a 4-parameter logistic curve to obtain the EC50 values for each compound. Generally, concentration-response data were based on 8 to 11 concentrations measured in duplicate. Serial dilutions of compound and positive controls were prepared in PBS containing 1% of BSA to prevent adsorption to lab plastics. As cytokine controls, recombinant human IL4 (Proteintech, Cat #HZ 1004), recombinant human IL13 (Invitrogen Cat #A42525), and recombinant human IL10 (Proteintech, Cat #HZ 1145) were used.

On overview of the cytokine receptor bioactivity data on the IL4 (type II) receptor and the IL10 receptor are provided as average EC50 values (in pM) in TABLE 13.

TABLE 13

| | HEK-Blue | HEK-Blue | SEQ |
|---|---|---|---|
| Description | IL4/13 | IL10 | ID NOs |
| IL4-10 fusion | 4.85 | 28.9 | 119 |
| (wt, dimer) | | | |
| IL4-13 fusion | 41.3 | | 120 |
| IL10M(J) | | 63.4 | 8 |
| IL10M(S) | | 149 | 3 |
| IL4-10M(J) | 12.7 | 77.8 | 67 |
| IL4-10M(S) | 44.6 | 117 | 66 |
| IL10M-Fc (J) | | 80.8 | 111 |
| IL4-Fc | 20.1 | | 108 |
| IL4-10M Fc (J) | 231 | 544 | 108, 111 |
| IL4-10M Fc (S) | 614 | 265 | 108, 110 |
| IL4-10M(J) | 137 | 520 | 183, 185 |
| mouse Fc | | | |
| IL4-13 Fc | 176 | | 108, 109 |
| IL4 | 5.02 | Negative | 10 |
| IL10 | Negative | 26.4 | 2 |
| IL13 | 33.0 | | 14 |

IL4-containing molecules all fully activated the HEK-Blue IL4/13 reporter cell line activity. The connection of an additional protein structure can lead to a reduction in potency, which in some embodiments is associated with the size of the protein connected. The IL4-10 dimer is most potent, without wishing to be bound by any particular theory the ability to form dimers may result in two IL4 polypeptides that can bind to receptors.

IL10-containing molecules also fully activated the HEK-Blue IL10 receptor cell line, and molecules containing the wild type (dimer-forming) version of IL10 exhibited higher potency. The connection of an additional protein structure can lead to a reduction in potency.

Surprisingly, an engineered IL10 polypeptide with rearranged helices as disclosed herein (IL10M(S)) was fully active and exhibited only a moderate reduction in potency compared to the IL10 engineered to be monomeric via a small insertion between helices D and E (IL10M(J)), despite the rearranged helices and the more extensive changes to the N-to-C terminal sequence relative to wild type IL10.

### L. Example 4: Heterologous Receptor Clustering

This example describes evaluation of heterologous clustering in response to fusion polypeptides or engineered immunocytokines disclosed herein. Heterologous receptor clustering can comprise, for example, binding and bringing two cytokine receptors or cytokine receptor subunits into close proximity, thereby inducing a unique signaling profile that is different than that induced by an individual cytokine or a combination of separate cytokines (e.g., corresponding cytokines that are not joined together, not joined to an immunoglobulin constant domain, or not present in a heterodimeric engineered immunocytokine).

Analysis of ligand-mediated heterologous receptor clustering was performed using a custom enzyme complementation bioluminescence-based protein-protein interaction assay based on the NanoBiTR reporter system (Promega). The assay uses an inactive version of NanoLuc (LgBiT) which becomes activated via complementation of small peptide (SmBiT). Both Enzyme fragments are connected to two proteins of interest via standard cloning methods and co-expressed in an expression system.

Proximity of the proteins of interest results in NanoLuc activation via the enzyme fragment complementation. This NanoLuc activation induces substrate conversion resulting in a bioluminescent signal. This technology was applied to measure proximity of cytokine receptors upon treatment with fusion polypeptides and engineered immunocytokines disclosed herein. Extracellular and transmembrane domains of different cytokine receptor chains were cloned into vectors provided in the NanoBiT kit. Two expression vectors, each cloned with a cytokine receptor, a linker, and either a SmBiT or LgBiT, were used to transiently transfect HEK293T cells and co-express two cytokine receptors. To measure the proximity of the cytokine receptors, transfected cells were loaded with a cell permeable substrate, furimazine, while monitoring the NanoLuc-induced bioluminescence on a plate reader at 37° C. Fifteen minutes after furimazine addition, compounds were added to the plate and bioluminescence measured for 30 minutes at 37° C.

For analysis, luminescence traces were normalized to the baseline luminescence response to reduce variability due to differences in cell numbers per well. Subsequently, signals were blank-corrected by subtracting the average signal after vehicle control addition. Blank corrected traces were further baseline corrected by subtracting the average of the 6 minutes prior to compound addition. The compound-induced peak luminescence values were determined based on a 3-point moving average and were subsequently fit with a 4-parameters logistic regression curve to determine the EC50.

For IL4 and IL10-containing fusion polypeptides and engineered heterodimeric immunocytokines, heterologous clustering was measured for the human IL4 receptor alpha (hIL4Ra) and the human IL10 receptor alpha (hIL10RA). For human IL4 and IL13-containing fusion polypeptides and engineered heterodimeric immunocytokines, heterologous clustering was measured for hIL4Ra and the human IL13 receptor alpha2 (hIL13Ra2). For mouse IL4 and IL13-containing fusion polypeptides and engineered heterodimeric immunocytokines, heterologous clustering was measured for mouse IL4Ra (mIL4Ra) and mouse IL13 receptor alpha2 (mIL13Ra2). For human IL13 and IL10-containing engineered heterodimeric immunocytokines, heterologous clustering was measured for the hIL10RA and the human IL13 receptor alpha1 (hIL13Rα1) or hIL13Ra2.

Fusion polypeptides and engineered immunocytokines showed concentration-dependent heterologous clustering of their cytokine-specific receptor chains which are summarized as average EC50 values (in pM) in TABLE 14, TABLE 15, and TABLE 16.

TABLE 14

| | Heterologous | |
|---|---|---|
| | clustering | |
| | hIL4Ra/hIL10RA | |
| | EC50 (2-7 | SEQ ID |
| Construct(s) | replicates) | NO(S) |
| IL4-10 fusion (wt, dimer) | 70.9 | 119 |
| IL4-10M(J) | 66.4 | 67 |
| IL4-10M (S) | 67.3 | 66 |
| IL4-10M Fc (J) | 201 | 108, 111 |
| IL4-10M Fc (S) | 221 | 108, 110 |
| IL4-10M(J) mouse Fc | 223 | 183, 185 |
| IL4 | Negative | 10 |
| IL10 | Negative | 2 |
| IL4 + IL10 | Negative | 10, 2 |

TABLE 15

| Heterologous | | | SEQ ID |
|---|---|---|---|
| clustering receptors | Construct(s) | EC50 | NO(S) |
| hIL4Ra/hIL13Ra2 | IL4-13 | 67.8 | 120 |
| | IL4-13 Fc | 188 | 108, 109 |
| | IL4 + IL13 | negative | 10, 14 |
| mIL4Ra/mIL13Ra2 | IL4-13 (mouse) | 187 | 197 |
| | IL4-13 Fc (mouse) | 1099 | 195, 182 |

TABLE 16

| Heterologous | | | SEQ ID |
|---|---|---|---|
| clustering receptors | Construct(s) | EC50 | NO(S) |
| hIL10RA/hIL13Ra1 | IL13-10M Fc (J) | 13870 | 180, 194 |
| | IL13 + IL10 | Negative | 14, 2 |
| hIL10RA/hIL13Ra2 | IL13-10M Fc (J) | 1489 | 180, 194 |
| | IL13 + IL10 | Negative | 14, 2 |

The individual cytokines, either alone or in combination, did not induce heterologous receptor clustering. Surprisingly, fusion polypeptides containing monomer versions of IL10 (e.g., engineered IL10 polypeptides disclosed herein) had comparable potencies to the version containing a dimer version of IL10 (e. g., wild type IL10).

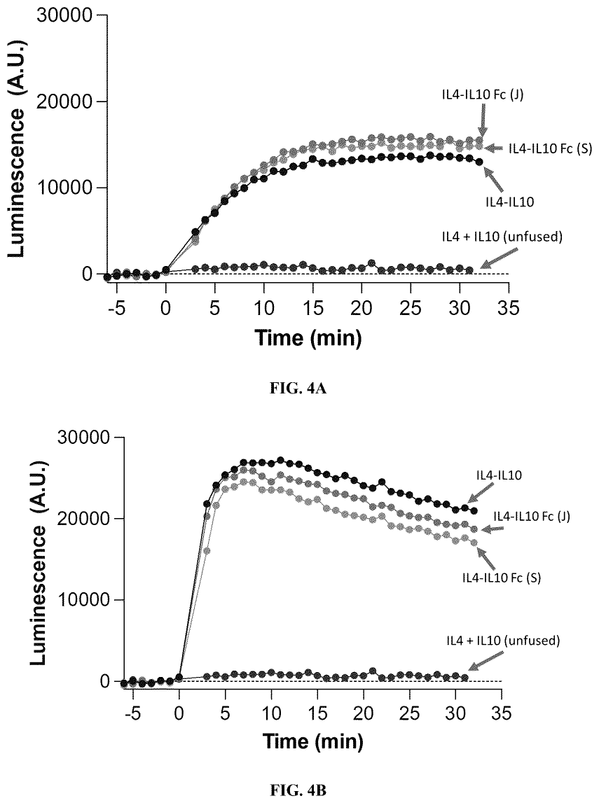

Temporal kinetics of ligand-mediated heterologous clustering showed concentration-dependent increases in the rate of clustering. Surprisingly, kinetics were highly comparable between a polypeptide fusion comprising IL4 and dimeric IL10 (e.g., wild type) within the same polypeptide chain, versus heterodimeric immunocytokines comprising IL4 and a monomeric version of IL10 separately linked to the N-termini of Fe heavy chains (FIG. 4A and FIG. 4B), suggesting that the monomeric variant has comparable receptor interaction when combined with a second cytokine.

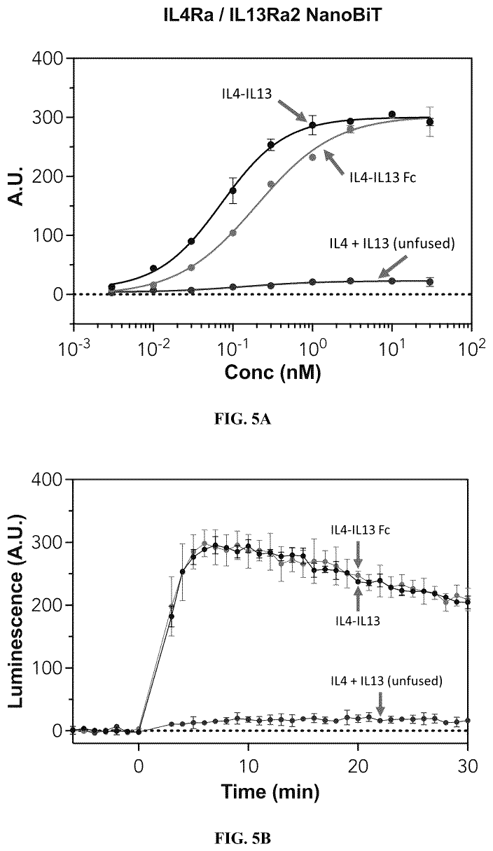

Kinetics were also comparable between a polypeptide fusion comprising IL4 and IL13 within the same polypeptide chain, versus heterodimeric immunocytokines comprising IL4 and IL13 separately linked to the N-termini of Fc heavy chains (FIG. 5A and FIG. 5B).

Compared to the receptor activity data of Example 3, the engineered immunocytokines (comprising Fc domains) showed smaller differences in receptor crosslinking potency versus the smaller single chain fusion polypeptides. Without wishing to be bound by any particular theory, in some embodiments the relative retention of receptor clustering activity can reflect bi-specific binding in the presence of both target cytokine receptors as compared to the reporter cell lines, in which only one of the two receptors can be present. For example, in some embodiments activity of the engineered immunocytokines in inducing signaling or eliciting a therapeutic effect in vivo can be higher than would be predicted based on the single receptor-based reporter assay. In some embodiments, the ability of an engineered heterodimeric immunocytokine disclosed herein to preferentially induce signaling in cells co-expressing a pair of cytokine receptors rather than just one (e.g., IL4 receptor and IL10 receptor, IL4 receptor and IL13 receptor, or IL10 receptor and IL13 receptor) can result in an advantageous efficacy and/or toxicity profile in vivo.

### M. Example 5: Pharmacokinetics

A rodent pharmacokinetic assay is performed to evaluate the half-lives of fusion polypeptides and engineered immunocytokines disclosed herein.

The fusion polypeptides and engineered immunocytokines (e.g., comprising murine cytokines and murine immunoglobulin constant domains) are administered to mice via administration route(s) disclosed herein (e.g., intravenous).

In another experiment, to test molecules with human amino acid sequences while minimizing the impact of anti-drug antibodies, human versions of the engineered immunocytokines are administered in an immunodeficient mouse model (e.g. SCID mice) or humanized mice (e.g., human FcRn transgenic mice) via administration route(s) disclosed herein (e.g., intravenous).

Blood samples are collected at regular intervals, and the fusion polypeptides/engineered immunocytokines are quantified in the blood, e.g., via an ELISA. Half-life and other pharmacokinetic parameters are calculated. Constructs are identified that have desirable pharmacokinetic profiles in systemic circulation.

### N. Example 6: MitoSOX Assay

Dorsal root ganglia of adult mice are dissected, and neurons are isolated by enzymatic digestion in Ca²⁺ and Mg²⁺ free HBSS containing 5 mM HEPES, 10 mM glucose, collagenase type XI (5 mg/ml) and dispase (10 mg/ml) for 1 h before mechanical trituration in DMEM containing 10% heat-inactivated fetal bovine serum. Cells are centrifuged for 5 min at 800 rpm, resuspended in DMEM containing 4.5 g/l glucose, 4 mM L-glutamine, 110 mg/l sodium pyruvate, 10% fetal bovine serum, 1% penicillin-streptomycin (10,000 i.u./ml), 1% glutamax, and 125 ng/ml nerve growth factor, and plated on glass cover slips coated with poly-L-lysine (0.01 mg/ml) and laminin (0.02 mg/ml). Neurons are cultured in an incubator set to 37° C. in 5% CO₂ and used 24 h after plating.

To induce increased reactive oxygen species (ROS) neuronal cultures are treated with Oxaliplatin (5 μg/ml). The cells are treated with fusion polypeptides or engineered immunocytokines disclosed herein. ROS production is measured using a MitoSOX superoxide indicator, with the intensity quantified using fluorescence microscopy. Constructs are identified that reduce ROS formation.

In some embodiments, a reduction in ROS indicates suitability of a fusion polypeptide or engineered immunocytokine for treating a disorder associated with mitochondrial dysfunction, such as a neurodegenerative disorder, diabetes, a musculoskeletal disorder, or a cardiovascular disorder.

### O. Example 7: In Vivo Efficacy

Persistent chemotherapy-induced polyneuropathy (CIPN) is induced in C57BL/6 mice by two 5-day periods of once-daily Oxaliplatin injections (3 mg/kg, i.p.) with an in-between 5-day dosing interruption. Mechanical hypersensitivity is measured at baseline and during the induction with Oxaliplatin using the von Frey test applying the up-down method described by Chaplan (1994). Following induction, animals are treated with (e.g., systemically administered) fusion polypeptides or engineered immunocytokines and changes in mechanical hypersensitivity are followed at repeated time points. Effects of treatment are determined by comparison to a vehicle-treated group. Constructs are compared for their magnitude and duration of mechanical hypersensitivity alleviation.

### Additional Sequences

TABLE 17

| SEQ | | |
|---|---|---|
| ID NO: | Description | Sequence |
| 119 | IL4-IL10 | HKCDITLQEIIKTLNSLTEQKTLCTELTVTDIFAASKNTTEKETF |
| | fusion | CRAATVLRQFYSHHEKDTRCLGATAQQFHRHKQLIRFLKRLD |
| | | RNLWGLAGLNSCPVKEANQSTLENFLERLKTIMREKYSKCSSG |
| | | SGGGGSGTSPGQGTQSENSCTHFPGNLPNMLRDLRDAFSRVKT |
| | | FFQMKDQLDNLLLKESLLEDFKGYLGCQALSEMIQFYLEEVMP |
| | | QAENQDPDIKAHVNSLGENLKTLRLRLRRCHRFLPCENKSKAV |
| | | EQVKNAFNKLQEKGIYKAMSEFDIFINYIEAYMTMKIRN |
| 120 | IL4-IL 13 | HHHHHHHKCDITLQEIIKTLNSLTEQKTLCTELTVTDIFAASKN |
| | fusion | TTEKETFCRAATVLRQFYSHHEKDTRCLGATAQQFHRHKQLIR |
| | (w/His tag) | FLKRLDRNLWGLAGLNSCPVKEANQSTLENFLERLKTIMREKY |
| | | SKCSSGSGGGGSGTSPGPVPPSTALRELIEELVNITQNQKAPLC |
| | | NGSMVWSINLTAGMYCAALESLINVSGCSAIEKTQRMLSGFCP |
| | | HKVSAGQFSSLHVRDTKIEVAQFVKDLLLHLKKLFREGRFN |
| 121 | Signal | MGWTLVFLFLLSVTAGVHS |
| | peptide | |
| 122 | Signal | MVSSAQFLGLLLLCFQGTRC |
| | peptide | |
| 188 | Signal | MGWSCIILFLVATATGVHS |
| | peptide | |
| 189 | Signal | MACPGFLWALVISTCLEFSMA |
| | peptide | |
| 123 | 6xHis tag | HHHHHH |
| 124 | Linker or | GGGSGG |
| | insertion | |
| 190 | Murine IL4 | CDKNHLREIIGILNEVTGEGTPCTEMDVPNVLTATKNTTESELV |
| | | CRASKVLRIFYLKHGKTPCLKKNSSVLMELQRLFRAFRCLDSSI |
| | | SCTMNESKSTSLKDFLESLKSIMQMDYS |
| 196 | Murine IL4 | HIHGCDKNHLREIIGILNEVTGEGTPCTEMDVPNVLTATKNTTE |
| | | SELVCRASKVLRIFYLKHGKTPCLKKNSSVLMELQRLFRAFRC |
| | | LDSSISCTMNESKSTSLKDFLESLKSIMQMDYS |
| 191 | Murine IL13 | APGPVPRSVSLPLTLKELIEELSNITQDQTPLCNGSMVWSVDLA |
| | | AGGFCVALDSLTNISNCNAIYRTQRILHGLCNRKAPTTVSSLPD |
| | | TKIEVAHFITKLLSYTKQLFRHGPF |
| 197 | Murine IL4- | HHHHHHIHGCDKNHLREIIGILNEVTGEGTPCTEMDVPNVLTA |
| | 13 fusion | TKNTTESELVCRASKVLRIFYLKHGKTPCLKKNSSVLMELQRL |
| | (w/His tag) | FRAFRCLDSSISCTMNESKSTSLKDFLESLKSIMQMDYSGSGGG |
| | | GSGTAPGPVPRSVSLPLTLKELIEELSNITQDQTPLCNGSMVWS |
| | | VDLAAGGFCVALDSLTNISNCNAIYRTQRILHGLCNRKAPTTV |
| | | SSLPDTKIEVAHFITKLLSYTKQLFRHGPF |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An engineered immunocytokine comprising:
(a) a first polypeptide chain comprising a first immunoglobulin heavy chain constant domain and IL4, wherein a C-terminus of the IL4 is joined to an N-terminus of the first immunoglobulin heavy chain constant domain via a first linker; and
(b) a second polypeptide chain comprising a second immunoglobulin heavy chain constant domain and a second cytokine, wherein the second cytokine is IL10 or IL13, wherein a C-terminus of the second cytokine is joined to an N-terminus of the second immunoglobulin heavy chain constant domain via a second linker.

2. The engineered immunocytokine of claim 1, wherein the second cytokine is the IL10.

3. The engineered immunocytokine of claim 2, wherein the IL10 is an engineered IL10 polypeptide comprising, from N-to-C terminus, an E helix, an F helix, an A helix, a B helix, a C helix, and a D helix.

4. The engineered immunocytokine of claim 3, wherein the engineered IL10 polypeptide is engineered to reduce or substantially eliminate formation of an IL10 homodimer.

5. The engineered immunocytokine of claim 3 or claim 4, wherein the engineered IL10 polypeptide comprises an amino acid sequence with at least 90% sequence identity to any one of SEQ ID NOs: 3-6.

6. The engineered immunocytokine of claim 3 or claim 4, wherein the engineered IL10 polypeptide comprises an amino acid sequence with at least 95% sequence identity to any one of SEQ ID NOs: 3-6.

7. The engineered immunocytokine of claim 3 or claim 4, wherein the engineered IL10 polypeptide comprises the amino acid sequence of any one of SEQ ID NOs: 3-6.

8. The engineered immunocytokine of claim 3 or claim 4, wherein the engineered IL10 polypeptide consists essentially of the amino acid sequence of any one of SEQ ID NOs: 3-6.

9. The engineered immunocytokine of any one of claims 1-8, wherein the first polypeptide chain comprises an amino acid sequence with at least 90% sequence identity to any one of SEQ ID Nos: 108, 113, and 179.

10. The engineered immunocytokine of any one of claims 1-8, wherein the first polypeptide chain comprises the amino acid sequence of any one of SEQ ID Nos: 108, 113, and 179.

11. The engineered immunocytokine of any one of claims 1-10, wherein the second cytokine is the IL10 and the second polypeptide chain comprises an amino acid sequence with at least 90% sequence identity to SEQ ID NO: 110 or 116.

12. The engineered immunocytokine of any one of claims 1-10, wherein the second cytokine is the IL10 and the second polypeptide chain comprises the amino acid sequence of SEQ ID NO: 110 or 116.

13. The engineered immunocytokine of claim 1, wherein the second cytokine is the IL10 and the second polypeptide chain comprises an amino acid sequence with at least 90% sequence identity to any one of SEQ ID NOs: 111, 118, and 194.

14. The engineered immunocytokine of claim 1, wherein the second cytokine is the IL10 and the second polypeptide chain comprises the amino acid sequence of any one of SEQ ID NOs: 111, 118, and 194.

15. The engineered immunocytokine of claim 1, wherein the second cytokine is the IL13.

16. The engineered immunocytokine of claim 1, wherein the second cytokine is the IL13 and the second polypeptide chain comprises an amino acid sequence with at least 90% sequence identity to any one of SEQ ID NOs: 109, 115, and 180.

17. The engineered immunocytokine of claim 1, wherein the second cytokine is the IL13 and the second polypeptide chain comprises the amino acid sequence of any one of SEQ ID NOs: 109, 115, and 180.

18. The engineered immunocytokine of any one of claims 1-17, wherein the first immunoglobulin heavy chain constant domain and the second immunoglobulin heavy chain each comprise a CH2 domain and a CH3 domain.

19. The engineered immunocytokine of any one of claims 1-18, wherein the first polypeptide chain and the second polypeptide chain each comprise a heterodimerization domain.

20. An engineered IL10 polypeptide comprising an A helix, an F helix, and a linker, wherein the A helix is joined to the F helix via the linker, and the engineered IL10 polypeptide is in a monomeric form.

21. An engineered IL10 polypeptide comprising a D helix and an E helix, wherein a C-terminus of the D helix is not joined to an N-terminus of the E helix.

22. An engineered IL10 polypeptide comprising, from N-to-C terminus, an E helix, an F helix, an A helix, a B helix, a C helix, and a D helix, wherein the engineered IL10 polypeptide is engineered to reduce or substantially eliminate formation of an IL10 homodimer.

23. The engineered IL10 polypeptide of claim 21 or claim 22, further comprising a linker.

24. The engineered IL10 polypeptide of claim 20 or claim 23, wherein an N-terminus of the A helix is joined to a C-terminus of the F helix via the linker.

25. The engineered IL10 polypeptide of any one of claims 20, 23, and 24, wherein the linker comprises the amino acid sequence of any one of SEQ ID NOs: 22-64.

26. The engineered IL10 polypeptide of claim 20 or claim 22, wherein the engineered IL10 polypeptide does not contain a second A helix or a second F helix.

27. The engineered IL10 polypeptide of claim 21 or claim 22, wherein the engineered IL10 polypeptide does not contain a second D helix or a second E helix.

28. The engineered IL10 polypeptide of claim 22, wherein the engineered IL10 polypeptide does not contain a second B helix or a second C helix.

29. The engineered IL10 polypeptide of any one of claims 20-21 and 23-28, wherein the engineered IL10 polypeptide is engineered to reduce or substantially eliminate formation of an IL10 homodimer.

30. The engineered IL10 polypeptide of any one of claims 20-29, wherein the engineered IL10 polypeptide comprises an amino acid sequence with at least 90% sequence identity to any one of SEQ ID NOs: 3-6.

31. The engineered IL10 polypeptide of any one of claims 20-29, wherein the engineered IL10 polypeptide comprises an amino acid sequence with at least 95% sequence identity to any one of SEQ ID NOs: 3-6.

32. The engineered IL10 polypeptide of any one of claims 20-29, wherein the engineered IL10 polypeptide comprises the amino acid sequence of any one of SEQ ID NOs: 3-6.

33. The engineered IL10 polypeptide of any one of claims 20-29, wherein the engineered IL10 polypeptide consists essentially of the amino acid sequence of any one of SEQ ID NOs: 3-6.

34. The engineered IL10 polypeptide of any one of claims 20-29, wherein the helices of the engineered IL10 are helices of mammalian IL10 or variant thereof.

35. The engineered IL10 polypeptide of any one of claims 20-29, wherein the helices of the engineered IL10 are helices of human IL10 or variant thereof.

36. A fusion polypeptide comprising an immunoglobulin constant domain and the engineered IL10 polypeptide of any one of claims 20-35.

37. The fusion polypeptide of claim 36, wherein the immunoglobulin constant domain comprises a heavy chain constant domain.

38. The fusion polypeptide of claim 37, wherein the heavy chain constant domain comprises a mammalian heavy chain constant domain.

39. The fusion polypeptide of claim 37, wherein the heavy chain constant domain comprises a human heavy chain constant domain.

40. The fusion polypeptide of claim 37, wherein the heavy chain constant domain comprises a human IgG1 CH2 domain and CH3 domain.

41. The fusion polypeptide of claim 37, wherein the heavy chain constant domain comprises a human IgG4 CH2 domain and CH3 domain.

42. The fusion polypeptide of claim 37, wherein the heavy chain constant domain comprises a human IgA CH2 domain and CH3 domain.

43. The fusion polypeptide of any one of claims 36-42, wherein the immunoglobulin constant domain comprises a light chain constant domain.

44. The fusion polypeptide of any one of claims 36-42, wherein the fusion polypeptide comprises an amino acid sequence with at least 86% sequence identity to SEQ ID NO: 110 or SEQ ID NO: 116.

45. The fusion polypeptide of any one of claims 36-42, wherein the fusion polypeptide comprises an amino acid sequence with at least 90% sequence identity to SEQ ID NO: 110 or SEQ ID NO: 116.

46. The fusion polypeptide of any one of claims 36-42, wherein the fusion polypeptide comprises an amino acid sequence with at least 95% sequence identity to SEQ ID NO: 110 or SEQ ID NO: 116.

47. The fusion polypeptide of any one of claims 36-42, wherein the fusion polypeptide comprises the amino acid sequence of SEQ ID NO: 110 or SEQ ID NO: 116.

48. A fusion polypeptide comprising the engineered IL10 polypeptide of any one of claims 20-35 and a second cytokine.

49. The fusion polypeptide of claim 48, wherein the second cytokine comprises an interleukin 4 (IL4) polypeptide.

50. The fusion polypeptide of claim 49, wherein the IL4 polypeptide comprises an amino acid sequence with at least 90% sequence identity to any one of SEQ ID NOs: 10-13.

51. The fusion polypeptide of claim 49, wherein the IL4 polypeptide comprises an amino acid sequence with at least 95% sequence identity to any one of SEQ ID NOs: 10-13.

52. The fusion polypeptide of claim 49, wherein the IL4 polypeptide comprises the amino acid sequence of any one of SEQ ID NOs: 10-13.

53. The fusion polypeptide of any one of claims 48-52, wherein the fusion polypeptide comprises an amino acid sequence with at least 65% sequence identity to SEQ ID NO: 65.

54. The fusion polypeptide of any one of claims 48-52, wherein the fusion polypeptide comprises an amino acid sequence with at least 80% sequence identity to SEQ ID NO: 65.

55. The fusion polypeptide of any one of claims 48-52, wherein the fusion polypeptide comprises an amino acid sequence with at least 90% sequence identity to SEQ ID NO: 65.

56. The fusion polypeptide of any one of claims 48-52, wherein the fusion polypeptide comprises the amino acid sequence of SEQ ID NO: 65.

57. The fusion polypeptide of claim 48, wherein the second cytokine comprises an interleukin 13 (IL13) polypeptide.

58. The fusion polypeptide of claim 57, wherein the IL13 polypeptide comprises an amino acid sequence with at least 90% sequence identity to any one of SEQ ID NOs: 14-21.

59. The fusion polypeptide of claim 57, wherein the IL13 polypeptide comprises an amino acid sequence with at least 95% sequence identity to any one of SEQ ID NOs: 14-21.

60. The fusion polypeptide of claim 57, wherein the IL13 polypeptide comprises the amino acid sequence of any one of SEQ ID NOs: 14-21.

61. The fusion polypeptide of any one of claims 57-60, wherein the fusion polypeptide comprises an amino acid sequence with at least 80% sequence identity to any one of SEQ ID NOs: 71-74.

62. An engineered immunocytokine comprising:
(a) a first polypeptide chain comprising a first immunoglobulin heavy chain constant domain and a first cytokine, wherein the first cytokine is an IL4 polypeptide, IL10 polypeptide, or IL13 polypeptide; and
(b) a second polypeptide chain comprising a second immunoglobulin heavy chain constant domain and a second cytokine, wherein the first cytokine and the second cytokine are different.

63. The engineered immunocytokine of claim 62, wherein the first cytokine is the IL4 polypeptide.

64. The engineered immunocytokine of claim 62, wherein the first cytokine is the IL10 polypeptide.

65. The engineered immunocytokine of claim 62, wherein the first cytokine is the IL13 polypeptide.

66. The engineered immunocytokine of claim 62, wherein the first cytokine is the IL4 polypeptide and the second cytokine is the IL10 polypeptide.

67. The engineered immunocytokine of claim 62, wherein the first cytokine is the IL4 polypeptide and the second cytokine is the IL13 polypeptide.

68. The engineered immunocytokine of claim 62, wherein the first cytokine is the IL10 polypeptide and the second cytokine is the IL13 polypeptide.

69. The engineered immunocytokine of any one of claims 62-68, wherein the first cytokine is a wild type cytokine.

70. The engineered immunocytokine of any one of claims 62-69, wherein the second cytokine is a wild type cytokine.

71. The engineered immunocytokine of any one of claims 62-70, wherein the first cytokine is a mammalian cytokine.

72. The engineered immunocytokine of any one of claims 62-71, wherein the first cytokine is human cytokine.

73. The engineered immunocytokine of any one of claims 62-72, wherein the second cytokine is a mammalian cytokine.

74. The engineered immunocytokine of any one of claims 62-73, wherein the second cytokine is a human cytokine.

75. The engineered immunocytokine of any one of claims 63, 66, and 67, wherein the IL4 polypeptide comprises an amino acid sequence with at least about 90% sequence identity to any one of SEQ ID NOs: 10-13.

76. The engineered immunocytokine of claim 64, 66, or 68, wherein the IL10 polypeptide comprises an amino acid sequence with at least about 90% sequence identity to any one of SEQ ID NOs: 1-9.

77. The engineered immunocytokine of claim 65, 67, or 68, wherein the IL13 polypeptide comprises an amino acid sequence with at least about 90% sequence identity to any one of SEQ ID NOs: 14-21.

78. The engineered immunocytokine of any one of claims 62-77, wherein the first immunoglobulin heavy chain constant domain comprises a CH2 domain and a CH3 domain.

79. The engineered immunocytokine of any one of claims 62-78, wherein the second immunoglobulin heavy chain constant domain comprises a CH2 domain and a CH3 domain.

80. The engineered immunocytokine of any one of claims 62-79, wherein the first immunoglobulin heavy chain constant domain and the second immunoglobulin heavy chain constant domain each comprise comprises a human IgG CH2 domain and CH3 domain.

81. The engineered immunocytokine of any one of claims 62-80, wherein the engineered immunocytokine comprises an Fc region that comprises the first immunoglobulin heavy chain constant domain and the second immunoglobulin heavy chain constant domain.

82. The engineered immunocytokine of claim 81, wherein the Fc region comprises a modification that reduces Fc receptor-mediated effector function.

83. The engineered immunocytokine of claim 81, wherein the Fc region comprises a modification that reduces Fc receptor binding.

84. The engineered immunocytokine of any one of claims 62-83, wherein the first immunoglobulin heavy chain constant domain and the second immunoglobulin heavy chain constant domain form a covalently-linked heterodimer.

85. The engineered immunocytokine of any one of claims 62-83, wherein the first polypeptide chain and the second polypeptide chain each comprise a heterodimerization domain.

86. The engineered immunocytokine of any one of claims 62-84, wherein the engineered immunocytokine comprises a knob-in-hole interaction between the first immunoglobulin heavy chain constant domain and the second immunoglobulin heavy chain constant domain.

87. The engineered immunocytokine of any one of claims 62-86, wherein a C-terminus of the first cytokine is joined to an N-terminus of the first immunoglobulin heavy chain constant domain.

88. The engineered immunocytokine of any one of claims 62-87, wherein a C-terminus of the second cytokine is joined to an N-terminus of the second immunoglobulin heavy chain constant domain.

89. The engineered immunocytokine of claim 87, wherein the C-terminus of the first cytokine is joined to the N-terminus of the first immunoglobulin heavy chain constant domain via a linker that comprises the amino acid sequence of any one of SEQ ID NOs: 22-64.

90. The engineered immunocytokine of claim 88, wherein the C-terminus of the second cytokine is joined to the N-terminus of the second immunoglobulin heavy chain constant domain via a linker that comprises the amino acid sequence of any one of SEQ ID NOs: 22-64.

91. The engineered immunocytokine of any one of claims 62-90, wherein the first polypeptide chain comprises an amino acid sequence with at least 90% sequence identity to any one of SEQ ID NOs: 108-118, 179, 180, and 194.

92. The engineered immunocytokine of any one of claims 62-91, wherein the second polypeptide chain comprises an amino acid sequence with at least 90% sequence identity to any one of SEQ ID NOs: 108-118, 179, 180, and 194.

93. The engineered immunocytokine of any one of claims 62-92, wherein the first polypeptide chain comprises the amino acid sequence of any one of SEQ ID NOs: 108-118, 179, 180, and 194.

94. The engineered immunocytokine of any one of claims 62-93, wherein the second polypeptide chain comprises the amino acid sequence of any one of SEQ ID NOs: 108-118, 179, 180, and 194.

95. The engineered immunocytokine of any one of claims 62-94, wherein the engineered immunocytokine exhibits a plasma half-life of at least 2 hours after intravenous administration as determined by a rodent pharmacokinetic assay.

96. An engineered immunocytokine comprising:
(a) an IL4 receptor binding domain;
(b) an IL10 receptor binding domain or an IL13 receptor binding domain; and
(c) a stabilizing domain;
wherein the engineered immunocytokine exhibits a plasma half-life of at least two hours after intravenous administration as determined by a rodent pharmacokinetic assay.

97. The engineered immunocytokine of claim 96, wherein the stabilizing domain comprises an immunoglobulin heavy chain constant domain.

98. The engineered immunocytokine of claim 96 or claim 97, wherein the stabilizing domain comprises an immunoglobulin CH2 domain and CH3 domain.

99. The engineered immunocytokine of claim 96 or claim 97, wherein the stabilizing domain comprises a human IgG CH2 domain and CH3 domain.

100. The engineered immunocytokine of claim 96 or claim 97, wherein the stabilizing domain comprises a human IgG1 CH2 domain and CH3 domain.

101. The engineered immunocytokine of claim 96 or claim 97, wherein the stabilizing domain comprises a human IgG4 CH2 domain and CH3 domain.

102. The engineered immunocytokine of claim 96 or claim 97, wherein the stabilizing domain comprises a human IgA CH2 domain and CH3 domain.

103. The engineered immunocytokine of any one of claims 96-102, wherein the stabilizing domain comprises an immunoglobulin Fc domain.

104. The engineered immunocytokine of any one of claims 96-103, wherein the IL4 receptor binding domain comprises a mammalian IL4 or a receptor-binding fragment thereof.

105. The engineered immunocytokine of any one of claims 96-104, wherein the engineered immunocytokine comprises the IL10 receptor binding domain, wherein the IL10 receptor binding domain comprises a mammalian IL10 or a receptor-binding fragment thereof.

106. The engineered immunocytokine of any one of claims 96-104, wherein the engineered immunocytokine comprises the IL13 receptor binding domain, wherein the IL13 receptor binding domain comprises a mammalian IL13 or a receptor-binding fragment thereof.

107. The engineered immunocytokine of any one of claims 96-106, wherein the IL4 receptor binding domain activates IL4 receptor signaling, the IL10 receptor binding domain activates IL10 receptor signaling, and the IL13 receptor binding domain activates IL13 receptor signaling.

108. An engineered immunocytokine comprising:
(a) a first polypeptide chain comprising a first immunoglobulin heavy chain constant domain and a first cytokine; and
(b) a second polypeptide chain comprising a second immunoglobulin heavy chain constant domain and a second cytokine, wherein the first cytokine and the second cytokine are different.

109. The engineered immunocytokine of claim 108, wherein a C-terminus of the first cytokine is joined to an N-terminus of the first immunoglobulin heavy chain constant domain, optionally via a linker, and a C-terminus of the second cytokine is joined to an N-terminus of the second immunoglobulin heavy chain constant domain, optionally via a linker.

110. The engineered immunocytokine of claim 108, wherein an N-terminus of the first cytokine is joined to a C-terminus of the first immunoglobulin heavy chain constant domain, optionally via a linker, and an N-terminus of the second cytokine is joined to a C-terminus of the second immunoglobulin heavy chain constant domain, optionally via a linker.

111. An engineered polypeptide comprising an IL4 polypeptide joined to a first immunoglobulin heavy chain, wherein the first immunoglobulin heavy chain is engineered to heterodimerize with a second immunoglobulin heavy chain.

112. The engineered polypeptide of claim 111, wherein the first immunoglobulin heavy chain is engineered to heterodimerize with a second immunoglobulin heavy chain.

113. The engineered polypeptide of claim 111 or 112, wherein a C-terminus of the IL4 polypeptide is joined to an N-terminus of the first immunoglobulin heavy chain.

114. The engineered polypeptide of claim 111 or 112, wherein a C-terminus of the IL4 polypeptide is joined to an N-terminus of the first immunoglobulin heavy chain via a linker that comprises the amino acid sequence of any one of SEQ ID NOs: 22-64.

115. The engineered polypeptide of any one of claims 111-114, wherein the engineered polypeptide comprises an amino acid sequence with at least 90% sequence identity to SEQ ID NO: 108, 112, or 113.

116. The engineered polypeptide of any one of claims 111-114, wherein the engineered polypeptide comprises an amino acid sequence with at least 95% sequence identity to SEQ ID NO: 108, 112, or 113.

117. The engineered polypeptide of any one of claims 111-114, wherein the engineered polypeptide comprises the amino acid sequence of SEQ ID NO: 108, 112, or 113.

118. An engineered polypeptide comprising an IL10 polypeptide joined to a first immunoglobulin heavy chain, wherein:
(a) the IL10 polypeptide is engineered to reduce or substantially eliminate formation of an IL10 homodimer; and
(b) the first immunoglobulin heavy chain is engineered to heterodimerize with a second immunoglobulin heavy chain.

119. The engineered polypeptide of claim 118, wherein the IL10 polypeptide comprises an amino acid sequence with at least 90% sequence identity to any one of SEQ ID NOs: 3-9.

120. The engineered polypeptide of claim 118, wherein the IL10 polypeptide consists essentially of an amino acid sequence with at least 90% sequence identity to any one of SEQ ID NOs: 3-9.

121. The engineered polypeptide of any one of claims 118-120, wherein the first immunoglobulin heavy chain is engineered to heterodimerize with the second immunoglobulin heavy chain.

122. An engineered polypeptide comprising an amino acid sequence with at least 65% sequence identity to SEQ ID NO: 65.

123. The engineered polypeptide of claim 122, wherein the engineered polypeptide comprises an amino acid sequence with at least 90% sequence identity to SEQ ID NO: 65.

124. The engineered polypeptide of claim 122, wherein the engineered polypeptide comprises the amino acid sequence of SEQ ID NO: 65.

125. An engineered polypeptide comprising an amino acid sequence with at least 96% sequence identity to SEQ ID NO: 108.

126. The engineered polypeptide of claim 125, wherein the engineered polypeptide comprises the amino acid sequence of SEQ ID NO: 108 or 113.

127. An engineered polypeptide comprising an amino acid sequence with at least 98% sequence identity to SEQ ID NO: 109.

128. The engineered polypeptide of claim 127, wherein the engineered polypeptide comprises the amino acid sequence of SEQ ID NO: 109 or 115.

129. An engineered polypeptide comprising an amino acid sequence with at least 86% sequence identity to SEQ ID NO: 110 or 116.

130. The engineered polypeptide of claim 129, wherein the engineered polypeptide can amino acid sequence with at least 90% sequence identity to SEQ ID NO: 110 or 116.

131. The engineered polypeptide of claim 129, wherein the engineered polypeptide can amino acid sequence with at least 95% sequence identity to SEQ ID NO: 110 or 116.

132. The engineered polypeptide of claim 129, wherein the engineered polypeptide comprises the amino acid sequence of SEQ ID NO: 110 or 116.

133. An engineered polypeptide comprising an amino acid sequence with at least 98% sequence identity to SEQ ID NO: 111.

134. The engineered polypeptide of claim 133, wherein the engineered polypeptide comprises the amino acid sequence of SEQ ID NO: 111.

135. The engineered polypeptide of any one of claims 111-134, wherein the engineered polypeptide comprises an Fc region with a modification that reduces Fc receptor-mediated effector function.

136. A nucleic acid encoding the engineered IL10 polypeptide of any one of claims 20-35, the fusion polypeptide of any one of claims 36-61, the engineered immunocytokine of any one of claims 1-19 and 62-110, or the engineered polypeptide of any of claims 111-135.

137. A vector comprising the nucleic acid of claim 136.

138. A pharmaceutical composition comprising the engineered IL10 polypeptide of any one of claims 20-35 and a pharmaceutically-acceptable excipient, wherein at least 70% of the engineered IL10 polypeptide is in a monomeric form.

139. A pharmaceutical composition comprising the engineered IL10 polypeptide of any one of claims 20-35 and a pharmaceutically-acceptable excipient, wherein no more than about 30% of the engineered IL10 polypeptide is in a dimeric form.

140. A pharmaceutical composition comprising the fusion polypeptide of any one of claims 48-61 and a pharmaceutically-acceptable excipient, wherein at least 70% of the fusion polypeptide is in a monomeric form.

141. A pharmaceutical composition comprising the fusion polypeptide of any one of claims 48-61 and a pharmaceutically-acceptable excipient, wherein no more than about 30% of the fusion polypeptide is in a dimeric form.

142. A pharmaceutical composition comprising the fusion polypeptide of any one of claims 36-61 and a pharmaceutically-acceptable excipient, wherein at most 30% of the engineered IL10 polypeptide is in an IL10 homodimeric form.

143. A pharmaceutical composition comprising a pharmaceutically-acceptable excipient and the engineered IL10 polypeptide of any one of claims 20-35, the fusion polypeptide of any one of claims 36-61, the engineered immunocytokine of any one of claims 1-19 and 62-110, the engineered polypeptide of any of claims 111-135, the nucleic acid of claim 136, or the vector of claim 137.

144. A method of treating a condition in a subject in need thereof, the method comprising administering to the subject an effective amount of the engineered IL10 polypeptide of any one of claims 20-35, the fusion polypeptide of any one of claims 36-61, the engineered immunocytokine of any one of claims 1-19 and 62-110, the engineered polypeptide of any of claims 111-135, the nucleic acid of claim 136, the vector of claim 137, or the pharmaceutical composition of any one of claims 138-143.

145. The method of claim 144, wherein the condition comprises pain.

146. The method of claim 144, wherein the condition comprises chronic pain.

147. The method of claim 144, wherein the condition comprises neuropathic pain.

148. The method of claim 144, wherein the condition comprises inflammatory pain.

149. The method of claim 144, wherein the condition comprises inflammation.

150. The method of claim 144, wherein the condition comprises chronic inflammation.

151. The method of claim 144, wherein the condition is osteoarthritis.

152. The method of claim 144, wherein the condition comprises a neuropathy.

153. The method of claim 144, wherein the condition comprises a chemotherapy-induced neuropathy.

154. The method of claim 144, wherein the condition comprises neurodegeneration.

155. The method of claim 144, wherein the administering comprises systemic administration.

156. The method of claim 144, wherein the administering comprises intravenous administration.
